(19) United States
(12) Patent Application Publication
(10) Pub. No.: US 2024/0384056 A1
(43) Pub. Date: Nov. 21, 2024

(54) COMPOSITION AND PHOTOSENSITIVE COMPOSITION

(71) Applicant: TOKYO OHKA KOGYO CO., LTD., Kanagawa (JP)

(72) Inventors: Ryutaro SUGAWARA, Kanagawa (JP); Takuro ASABA, Kanagawa (JP)

(21) Appl. No.: 18/694,174

(22) PCT Filed: Aug. 18, 2022
(86) PCT No.: PCT/JP2022/031269

§ 371 Date: Mar. 21, 2024

(30) Foreign Application Priority Data
Sep. 22, 2021 (JP) 2021-154894

(51) Int. Cl. C08K 3/08; C07C 319/14; C07C 319/20; C07C 323/19; C07C 323/20; C08F 122/24; C08K 3/22

(52) U.S. Cl. CPC C08K 3/08; C07C 319/14; C07C 319/20; C07C 323/19; C07C 323/20; C08F 122/24; C08K 3/22; C08K 2003/0831; C08K 2003/2241; C08K 2003/2244

(57) ABSTRACT

A photosensitive composition which tends to hardly occur excessive decrease of weight of a component (in which is a component other than a solvent, when the composition or the photosensitive composition includes the solvent) in the composition or the photosensitive composition by heating, and include inorganic microparticles in a stably dispersed state, a cured product of the photosensitive composition, a compound which can be preferably added to the composition and the photosensitive composition, and a production method of the compound are provided. In a composition including a photopolymerizable compound (A) and inorganic microparticles (B) or a photosensitive composition including a photopolymerizable compound (A), inorganic microparticles (B), and an initiator, a compound with a specific structure having a radically polymerizable group-containing group or a cationically polymerizable group-containing group is used as the photopolymerizable compound (A).

### TECHNICAL FIELD

The present invention relates to a composition and a photosensitive composition including a photopolymerizable compound (A) and inorganic microparticles, a cured product of the photosensitive composition, a compound that can be preferably added to the photosensitive composition, and a production method of the compound.

### BACKGROUND ART

Heretofore, various inorganic microparticles are blended into compositions used to form materials with various functionalities for the purpose of imparting functionality to the materials. For example, for forming optical materials, highly refractive materials are used. A composition in which metal oxide particles such as titanium dioxide and zirconium oxide are dispersed is used as highly refractive material, for example. As a composition for forming such a highly refractive material, an energy beam curable composition including a metal oxide (A) with a specific particle size, (meth)acrylate (B), and a photoinitiator (C) has been proposed (see Patent Document 1).

### CITATION LIST

### Patent Document

Patent Document 1: Japanese Unexamined Patent Application, Publication No. 2017-214465

### DISCLOSURE OF THE INVENTION

### Problems to be Solved by the Invention

As described above, a material with high refractive index can be formed by using a composition described in the Patent Document 1. However, in case that a functional material such as a highly refractive material is formed by using a composition such that Patent Document 1 discloses, a weight of components in the composition other than solvent tend to excessively decrease, when the composition is heated during formation of the functional material. In addition, there is also a problem that stable dispersion of inorganic microparticles in the known composition such that Patent Document 1 discloses for a long period of time is difficult.

In light of the above problems, the present invention has been made and an object thereof is to provide a composition and a photosensitive composition which tend to hardly occur excessive decrease of weight of a component (in which is a component other than a solvent, when the composition or the photosensitive composition includes the solvent) in the composition or the photosensitive composition by heating, and include inorganic microparticles in a stably dispersed state, a cured product of the photosensitive composition, a compound which can be preferably added to the composition and the photosensitive composition, and a production method of the compound. Means for Solving the Problems

The present inventors have found that the above problems can be solved by using a compound with a specific structure having a radically polymerizable group-containing group or a cationically polymerizable group-containing group as a photopolymerizable compound (A) in a composition including the photopolymerizable compound (A) and inorganic microparticles (B) or a photosensitive composition including the photopolymerizable compound (A), inorganic microparticles (B), and an initiator. Thus, the present invention has been completed. Specifically, the present invention provides the followings.

A first aspect of the present invention is directed to a composition including a photopolymerizable compound (A), and inorganic microparticles (B),
in which, the photopolymerizable compound (A) includes a compound represented by following formula (A1):

R^{a01}—X^{a03}—R^{a02}—X^{a01}-Ph¹-S-Ph²-X^{a02}—R^{a04}—X^{a04}—R^{a03} (A1),
in which, in the formula (A1), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A1) does not simultaneously have the radically polymerizable group-containing group and the cationically polymerizable group-containing group.

A second aspect of the present invention is directed to a photosensitive composition including a photopolymerizable compound (A), inorganic microparticles (B), and an initiator (C),
in which, the photopolymerizable compound (A) includes a compound represented by following formula (A1):

R^{a01}—X^{a03}—R^{a02}—X^{a01}-Ph¹-S-Ph²-X^{a02}—R^{a04}—X^{a04}—R^{a03} (A1),
in which, in the formula (A), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A1) does not simultaneously have the radically polymerizable group-containing group and the cationically polymerizable group-containing group.

A third aspect of the present invention is directed to a cured product of the photosensitive composition according second aspect.

A fourth aspect of the present invention is directed to a compound represented by following formula (A1):

R^{a01}—X^{a03}—R^{a02}—X^{a01}-Ph¹-S-Ph²-X^{a02}—R^{a04}—X^{a04}—R^{a03} (A1)
in which, in the formula (A1), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A1) does not simultaneously have the radically polymerizable group-containing group and the cationically polymerizable group-containing group, and
provided that, when both of X^{a01} and X^{a02} are sulfur atom, X^{a01} bonds to para-position in Ph¹ relative to the sulfur atom bonding Ph¹ and Ph², and X^{a02} bonds to para-position in Ph² relative to the sulfur atom bonding Ph¹ and Ph², R^{a02} and R^{a04} are not a group represented by —CH₂CH₂—O—CH₂CH₂—.

A fifth aspect of the present invention is directed to a production method of the compound according to the fourth aspect including,
obtaining a compound represented by following formula (A1-d) by reacting a compound represented by following formula (A1-a), a compound represented by following formula (A1-b), and a compound represented by following formula (A1-c), and
substituting hydrogen atom in a terminus represented by —X^{a03}—H with a group represented by R^{a01}, and substituting hydrogen atom in a terminus represented by —X^{a04}—H with a group represented by R^{a03} in the compound represented by the formula (A1-d):

H—X^{a01}-Ph¹-S-Ph²-X^{a02}—H (A1-a)

H—X^{a03}—R^{a02}-Hal (A1-b)

H—X^{a04}—R^{a04}-Hal (A1-c)

H—X^{a03}—R^{a02}—X^{a01}-Ph¹-S-Ph²-X^{a02}—R^{a04}—X^{a04}—H (A1-d)
in which, in the formula (A1-a), the formula (A1-b), the formula (A1-c), and the formula (A1-d), R^{a01}, R^{a02}, R^{a04}, X^{a01} to X^{a04}, Ph¹, and Ph² are the same as these in the formula (A1), and Hal is a halogen atom.

A sixth aspect of the present invention is directed to a production method of the compound according to the fourth aspect including,
Obtaining the compound represented by the formula (A1) by reacting a compound represented by following formula (A1-a), a compound represented by following formula (A1-e), and a compound represented by following formula (A-lf) in presence of a base:

H—X^{a01}-Ph¹-S-Ph²-X^{a02}—H (A1-a)

R^{a01}—X^{a03}—R^{a02}-Hal (A1-e)

R^{a03}—X^{a04}—R^{a04}-Hal (A1-f)

in which, in the formula (A1-a), the formula (A1-e), and the formula (A1-f), R^{a01} to R^{a4}, X^{a01} to X^{a04}, Ph¹, and Ph² are the same as these in the formula (A1), and Hal is a halogen atom.

### Effects of the Invention

According to the present invention, it is possible to provide a composition and a photosensitive composition which tend to hardly occur excessive decrease of weight of a component other than a solvent by heating, and include inorganic microparticles in a stably dispersed state, a cured product of the photosensitive composition, a compound which can be preferably added to the composition and the photosensitive composition, and a production method of the compound.

### PREFERRED MODE FOR CARRYING OUT THE INVENTION

### <<Composition>>

A composition includes a photopolymerizable compound (A) and inorganic microparticles (B). The composition is substantially an inorganic microparticles dispersion composition. The composition is used for forming a functional material having a property corresponding to a type of the inorganic particles after addition of an initiator which cures the photopolymerizable compound (A). The photopolymerizable compound (A) includes a compound represented by following formula (A1). R^{a01}—X^{a03}—R^{a02}—X^{a01}-Ph¹-S-Ph²-X^{a02}—R^{a04}—X^{a04}—R^{a03} (A1)

In the formula (A1), R^{a01} and R^{a03} are each independently a radically polymerizable group-containing group or a cationically polymerizable group-containing group. R^{a02} and R^{a04} are each independently an alkylene group which may be interrupted by one or more oxygen atoms and/or one or more sulfur atoms. X^{a01}, X^{a02}, X^{a03}, and X^{a04} are each independently oxygen atom of sulfur atom. Ph¹ and Ph² are each independently a phenylene group optionally substituted with an alkyl group having 1 or more and 5 or less carbon atoms. Sum of a number of oxygen atoms and/or sulfur atoms included in the alkylene group as R^{a02} and a number of oxygen atoms and/or sulfur atoms included in the alkylene group as R^{a04} is 2 or more. The compound represented by the formula (A1) does not simultaneously have the radically polymerizable group-containing group and the cationically polymerizable group-containing group.

By inclusion of the compound represented by the above formula (A1), excessive decrease of the weight of the components other than the solvent is hardly to occur in case that the composition is heated, and the inorganic microparticles are stably dispersed in the composition for a long period of time.

Hereinafter, essential of optional components that may be included in the composition will be described below.

<Photopolymerizable compound (A)>

The composition includes a photopolymerizable compound (A). The photopolymerizable compound is a compound having a radically polymerizable group-containing group or a cationically polymerizable group-containing group.

As the radically polymerizable group-containing group, typically, a group having an ethylenically unsaturated double bond is exemplified. As the ethylenically unsaturated double bond-containing group, an alkenyl group-containing group containing an alkenyl group such as a vinyl group or an allyl group is preferred, and a (meth)acryloyl group is more preferred. Examples of the cationically polymerizable group typically include an epoxy group-containing group, an oxetanyl group-containing group, a vinyloxy group-containing group, and a vinylthio group-containing group. Among these, the epoxy-group containing group and the vinyloxy group-containing group are preferred. As the epoxy group-containing group, an alicyclic epoxy group-containing group and glycidyl group are preferred. It should be noted that the alicyclic epoxy group is an aliphatic cyclic group in which adjacent two carbon atoms, as atoms constituting the ring, are bonded via an oxygen atom. That is, the alicyclic epoxy group has an epoxy group including a 3-membered ring composed of two carbon atoms and one oxygen atom on the aliphatic ring.

In the present specification and the present claims, (meth)acrylic means both acrylic and methacrylic. (meth)acryloyl means both acryloyl and methacryloyl. (meth)acrylate means both acrylate and methacrylate.

### [Compound (A1)]

As described above, the photopolymerizable compound (A) includes the compound represented by the formula (A1).

R^{a01}—X^{a03}—R^{a02}—X^{a01}-Ph¹-S-Ph²-X^{a02}—R^{a04}—X^{a04}—R^{a03} (A1)

In the formula (A1), R^{a01} and R^{a03} are each independently a radically polymerizable group-containing group or a cationically polymerizable group-containing group. R^{a02} and R^{a04} are each independently an alkylene group which may be interrupted by one or more oxygen atoms and/or one or more sulfur atoms. X^{a01}, X^{a02}, X^{a03}, and X^{a04} are each independently oxygen atom of sulfur atom. Ph¹ and Ph² are each independently a phenylene group optionally substituted with an alkyl group having 1 or more and 5 or less carbon atoms. Sum of a number of oxygen atoms and/or sulfur atoms included in the alkylene group as R^{a02} and a number of oxygen atoms and/or sulfur atoms included in the alkylene group as R^{a4} is 2 or more. The compound represented by the formula (A1) does not simultaneously have the radically polymerizable group-containing group and the cationically polymerizable group-containing group.

In the present specification, the compound represented by the formula (A1) is referred to as “compound (A1)”

In the formula (A1), Ph¹ and Ph² are each independently a phenylene group optionally substituted with an alkyl group having 1 or more and 5 or less carbon atoms. The phenylene group as Ph¹ and Ph² may be any one of o-phenylene group, m-phenylene group, and p-phenylene group, and is preferably p-phenylene group.

The aromatic group as Ar^{a01} may be substituted with one or more groups selected from a group consisting of an alkyl group having 1 or more and 5 or less carbon atoms, cyano group, and a halogen atom. A number of alkyl groups as substituent bonding to phenylene group as Ph¹ and Ph² is not particularly limited. Examples of the alkyl group having 1 or more and 5 or less carbon atoms include methyl group, ethyl group, n-propyl group, isopropyl group, n-butyl group, isobutyl group, sec-butyl group, tert-butyl group, n-pentyl group, isopentyl group, neopentyl group, sec-pentyl group and tert-pentyl group. Among these group, methyl group and ethyl group are preferred.

R^{a01} and R^{a03} are each independently a radically polymerizable group-containing group or a cationically polymerizable group-containing group. The radically polymerizable group-containing group and the cationically polymerizable group-containing group are as described above. The radically polymerizable group-containing group as R^{a01} and R^{a03} is preferably a (meth)acryloyl group-containing group, and more preferably a (meth)acryloyl group. As the cationically polymerizable group-containing group as R^{a01} and R^{a03}, vinyl group, a vinyl oxy group-containing group, and an epoxy group-containing group are preferred, vinyl group, and the epoxy group-containing group are more preferred, and an alicyclic epoxy group-containing group and glycidyl group are preferred. Vinyl group is generally radically polymerizable group. However, when R^{a01} and R^{a03} is a vinyl group in the formula (A1), the vinyl group and X^{a03} or X^{a04} that is oxygen atom or sulfur atom forms vinyloxy group or vinylthio group that is cationically polymerizable group. Therefore, the vinyl group as R^{a01} and R^{a03} in the formula (A1) is defined as not radically polymerizable group but cationically polymerizable group. As the alicyclic epoxy group-containing group, an alicyclic epoxy group-containing group represented by formula (a1-IIIa) or formula (a1-IIIb) described below is preferred.

R^{a02} and R^{a4} are each independently an alkylene group which may be interrupted by one or more oxygen atoms and/or one or more sulfur atoms. R^{a02} and R^{a04} are preferably an alkylene group interrupted by one or more oxygen atoms and/or one or more sulfur atoms. Number of carbon atoms in the alkylene group which may be interrupted by one or more oxygen atoms and/or one or more sulfur atoms as R^{a02} and R^{a4} is not particularly limited as long as the desired effect is not impaired

The alkylene group which may be interrupted by one or more oxygen atoms and/or one or more sulfur atoms as R^{a02} and R^{a04} preferably consists of ma aliphatic chain saturated hydrocarbon groups selected from the alkylene group having 1 or more and 4 or less carbon atoms, the alkanetriyl group having 1 or more and 4 or less carbon atoms, and the alkyl group having 1 or more and 4 or less carbon atom, and (ma-1) oxygen atoms and/or sulfur atoms bridging the ma aliphatic chain hydrocarbon groups. Herein, ma is an integer of 2 or more and 6 or less.

Suitable examples of the alkylene group having 1 or more and 4 or less carbon atoms include methylene group, ethane-1,2-diyl group (ethylene group), propae-1,2-diyl group, propane-1,3-diyl group, butane-1,2-diyl group, butane-1,3-diyl group, and butane-1,4-diyl group. Among these groups, ethane-1,2-diyl group (ethylene group), propane-1,2-diyl group and propane-1,3-diyl group are preferred. Suitable examples of the alkanetriyl group having 1 or more and 4 or less carbon atoms include propane-1,2,3-triyl group, butane-1,2,3-triyl group, and butane-1,2,4-triyl group. Among these groups, propane-1,2,3-triyl group is preferred. Suitable examples of the alkyl group having 1 or more and 4 or less carbon atoms include methyl group, ethyl group, n-propyl group, isopropyl group, n-butyl group, isobutyl group, sec-butyl group, and tert-butyl group. Among these groups, methyl group and ethyl group are preferred.

The alkylene group which may be interrupted by one or more oxygen atoms and/or one or more sulfur atoms as R^{a02} and R^{a04} is preferably a group consisting of ma alkylene groups having 1 or more 4 or less carbon atoms, and (ma-1) oxygen atoms and/or sulfur atoms bridging the ma alkylene groups. Herein, ma is an integer of 2 or more and 6 or less.

Suitable specific examples of the alkylene group which may be interrupted by one or more oxygen atoms and/or one or more sulfur atoms as R^{a02} and R^{a04} include following groups.
—CH₂CH₂—O—CH₂CH₂—
—(CH₂CH₂—O)₂—CH₂CH₂—
—(CH₂CH₂—O)₃—CH₂CH₂—
—(CH₂CH₂—O)₄—CH₂CH₂—
—(CH₂CH₂—O)₅—CH₂CH₂—
—C(CH₃)HCH₂—O—C(CH₃)HCH₂—
—(C(CH₃)HCH₂—O)₂—C(CH₃)HCH₂—
—(C(CH₃)HCH₂—O)₃—C(CH₃)HCH₂—
—(C(CH₃)HCH₂—O)₄—C(CH₃)HCH₂—
—(C(CH₃)HCH₂—O)₅—C(CH₃)HCH₂—
—CH₂CH₂CH₂—O)—CH₂CH₂CH₂—
—(CH₂CH₂CH₂—O)₂—CH₂CH₂CH₂—
—(CH₂CH₂CH₂—O)₃—CH₂CH₂CH₂—
—(CH₂CH₂CH₂—O)₄—CH₂CH₂CH₂—
—(CH₂CH₂CH₂—O)₅—CH₂CH₂CH₂—
—CH₂C(OCH₃)HCH₂—
—CH₂C(OCH₂CH₃)HCH₂—
—CH₂C(OCH₂CH₂CH₃)HCH₂—
—CH₂C(OCH₂CH₂CH₂CH₃)HCH₂—
CH₂CH₂—S—CH₂CH₂—
—(CH₂CH₂—S)₂—CH₂CH₂—
—(CH₂CH₂—S)₃—CH₂CH₂—
—(CH₂CH₂—S)₄—CH₂CH₂—
—(CH₂CH₂—S)₅—CH₂CH₂—
—C(CH₃)HCH₂—S—C(CH₃)HCH₂—
—(C(CH₃)HCH₂—S) 2—C(CH₃)HCH₂—
—(C(CH₃)HCH₂—S)₃—C(CH₃)HCH₂—
—(C(CH₃)HCH₂—S) 4—C(CH₃)HCH₂—
—(C(CH₃)HCH₂—S)₅—C(CH₃)HCH₂—
—CH₂CH₂CH₂—S—CH₂CH₂CH₂—
—(CH₂CH₂CH₂—S)₂—CH₂CH₂CH₂—
—(CH₂CH₂CH₂—S)₃—CH₂CH₂CH₂—
—(CH₂CH₂CH₂—S)₄—CH₂CH₂CH₂—
—(CH₂CH₂CH₂—S)₅—CH₂CH₂CH₂—
—CH₂C(SCH₃)HCH₂—
—CH₂C(SCH₂CH₃)HCH₂—
—CH₂C(SCH₂CH₂CH₃)HCH₂—
—CH₂C(SCH₂CH₂CH₂CH₃)HCH₂—

Among these groups,
—CH₂CH₂—O—CH₂CH₂—,
—(CH₂CH₂—O)₂—CH₂CH₂—,
—(CH₂CH₂—O)₃—CH₂CH₂—,
—CH₂CH₂CH₂—O—CH₂CH₂CH₂—,
—CH₂CH₂—S—CH₂CH₂—,
—(CH₂CH₂—S)₂—CH₂CH₂—, and
—(CH₂CH₂—S)₃—CH₂CH₂— are preferred, and
—(CH₂CH₂—O)₂—CH₂CH₂—,
—(CH₂CH₂—O)₃—CH₂CH₂—,
—(CH₂CH₂—S)₂—CH₂CH₂—, and
—(CH₂CH₂—S)₃—CH₂CH₂— are more preferred.

In the formula (A1), X^{a01}, X^{a02}, X^{a03}, and X^{a04} are each independently oxygen atom of sulfur atom.

In the formula (A1), sum of a number of oxygen atoms and/or sulfur atoms included in the alkylene group as R^{a02} and a number of oxygen atoms and/or sulfur atoms included in the alkylene group as R^{a04} is 2 or more. By inclusion of oxygen atom and/or sulfur atom in an amount equal to or more than specific amount at predetermined part in the compound (A1), excessive loss of mass of the component other than the solvent is suppressed by heating the composition, and dispersion of the inorganic microparticles (B) in the composition can be stabilized. Upper limits of the number of oxygen atoms and/or sulfur atoms included in the alkylene group as R^{a02} and the number of oxygen atoms and/or sulfur atoms included in the alkylene group as R^{a04} are not particularly limited as long as the desired effect is not impaired. For example, sum of the number of oxygen atoms and/or sulfur atoms included in the alkylene group as R^{a02}, and the number of oxygen atoms and/or sulfur atoms included in the alkylene group as R^{a04} is preferably 2 or more and 10 or less, more preferably 2 or more and 8 or less, and further preferably 2 or more and 6 or less.

Suitable examples of the compound (A1) include following compounds. Compounds which a bridging group bridging acryloyloxy group or methacryloyloxy group and aryloxy group, arylthio group, heteroaryloxy group, or heteroarylthio group is replaced with —CH₂CH₂—O—CH₂CH₂—, —(CH₂CH₂—O)₂—CH₂CH₂—, —(CH₂CH₂—O)₃—CH₂CH₂—, —CH₂CH₂CH₂—O—CH₂CH₂CH₂—, —CH₂CH₂—S—CH₂CH₂—, —(CH₂CH₂—S)₂—CH₂CH₂—, and —(CH₂CH₂—S)₃—CH₂CH₂— in following compounds are preferred as the novel compound.

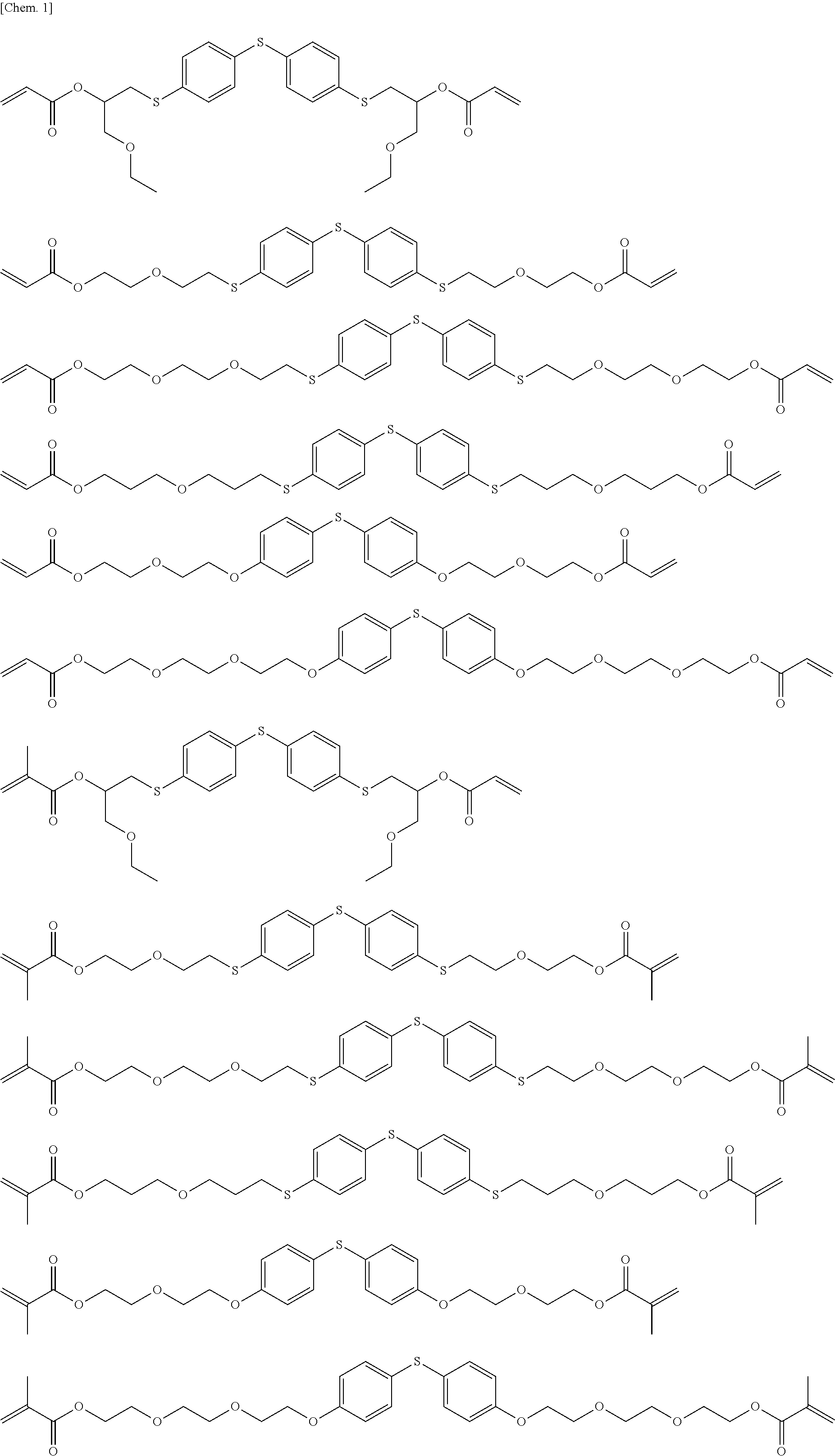

### [Novel Compound]

A compound represented by following formula (A1) is provided as a novel compound.

R^{a01}—X^{a03}—R^{a02}—X^{a01}-Ph¹-S-Ph²-X^{a02}—R^{a04}—Xₐ₀₄—R^{a03} (A1)

In the formula (A1), R^{a01} and R^{a03} are each independently a radically polymerizable group-containing group or a cationically polymerizable group-containing group. R^{a02} and R^{a4} are each independently an alkylene group which may be interrupted by one or more oxygen atoms and/or one or more sulfur atoms. X^{a01}, X^{a02}, X^{a03}, and X^{a04} are each independently oxygen atom of sulfur atom. Ph¹ and Ph² are each independently a phenylene group optionally substituted with an alkyl group having 1 or more and 5 or less carbon atoms. Sum of a number of oxygen atoms and/or sulfur atoms included in the alkylene group as R^{a02} and a number of oxygen atoms and/or sulfur atoms included in the alkylene group as R^{a04} is 2 or more. The compound represented by the formula (A1) does not simultaneously have the radically polymerizable group-containing group and the cationically polymerizable group-containing group. Provided that, when both of X^{a01} and X^{a02} are sulfur atom, X^{a01} bonds to para-position in Ph¹ relative to the sulfur atom bonding Ph¹ and Ph², and X^{a02} bonds to para-position in Ph² relative to the sulfur atom bonding Ph¹ and Ph², R^{a02} and R^{a04} are not a group represented by —CH₂CH₂—O—CH₂CH₂—.

The formula (A1) is described above.

Suitable examples of the novel compound include following compounds. Compounds which a bridging group bridging acryloyloxy group or methacryloyloxy group and aryloxy group, arylthio group, heteroaryloxy group, or heteroarylthio group is replaced with —CH₂CH₂—O—CH₂CH₂—, —(CH₂CH₂—O)₂—CH₂CH₂—, —(CH₂CH₂—O)₃—CH₂CH₂—, —CH₂CH₂CH₂—O—CH₂CH₂CH₂—, —CH₂CH₂—S—CH₂CH₂—, —(CH₂CH₂—S)₂—CH₂CH₂—, and —(CH₂CH₂—S)₃—CH₂CH₂— in following compounds are preferred as the novel compound.

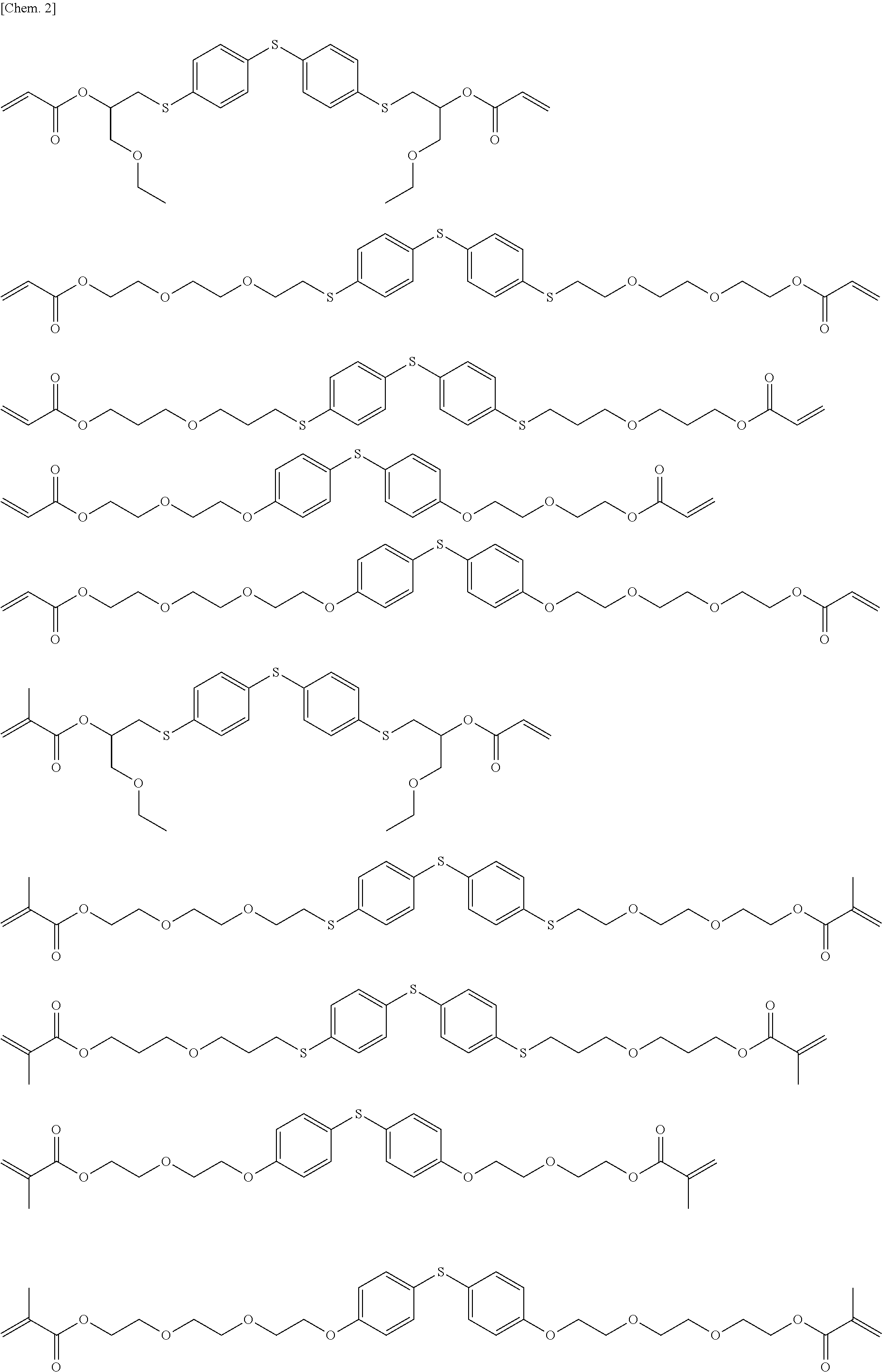

A production method of the compound represented by the formula (A1) is not particularly limited. As suitable production method, a method including,
obtaining a compound represented by following formula (A1-d) by reacting a compound represented by following formula (A1-a), a compound represented by following formula (A1-b), and a compound represented by following formula (A1-c), and
substituting hydrogen atom in a terminus represented by —X^{a03}—H with a group represented by R^{a01}, and substituting hydrogen atom in a terminus represented by —X^{a04}—H with a group represented by R^{a03} in the compound represented by the formula (A1-d),
is exemplified.

H—X^{a01}-Ph¹-S-Ph²-X^{a02}—H (A1-a)

H—X^{a03}—R^{a02}-Hal (A1-b)

H—X^{a04}—R^{a04}-Hal (A1-c)

H—X^{a03}—R^{a02}—X^{a01}-Ph¹-S-Ph²-X^{a02}—R^{a04}—X^{a04}—H (A1-d)

In the formula (A1-a), the formula (A1-b), the formula (A1-c), and the formula (A1-d), R^{a01}, R^{a02}, R^{a04}, X^{a01} to X^{a04}, Ph¹ and Ph² are the same as these in the formula (A1). Hal is a halogen atom.

A reaction of the compound represented by the formula (A1-a) and the compound represented by the formula (a1-1b) in presence of a base is usually carried out in presence an organic solvent. The organic solvent used for the reaction of the compound represented by the formula (A1-a) with the compound represented by the formula (A1-b) and the compound represented by the formula (A1-c) is not particularly limited as long as the organic solvent that does not inhibit the progress of the reaction. Since the reaction is carried out in presence of the base, as the organic solvent, an organic solvent not having an acidic group such as carboxy group, and sulfonic acid group, and hydroxy group is preferred. Since it is easy to proceed a reaction well, an aprotic polar organic solvent is preferred as the organic solvent. Suitable examples of the aprotic polar organic solvent include N,N-dimethylformamide, N,N-dimethylacetamide, N-methyl-2-pyrrolidone, dimethylsulfoxide, tetrahydrofuran, cyclopentyl methyl ether, acetonitrile, and hexamethylphosphoric triamide.

The compound represented by the formula (A1-a), the compound represented by the formula (A1-b), and the compound represented by the formula (A1-c) may simultaneously react. The compound represented by the formula (A1-b) and the compound represented by the formula (A1-c) may be reacted with the compound represented by the formula (A1-a) in any order. It is preferred that the compound represented by the formula (A1-b) and the compound represented by the formula (A1-c) are the same compound because of the ease of purification of the intermediate and the final product. When multiple compounds are formed by the reaction of the compound represented by the formula (A1-a) with the compound represented by the formula (A1-b) and the compound represented by the formula (A1-c), for example, various well-known chromatographic methods such as column chromatography can be used to separate the compound represented by the formula (A1-d).

An amount of the organic solvent in the reaction of the compound represented by the formula (A1-a), the compound represented by the formula (A1-b), and the compound represented by the formula (A1-c) is not particularly limited. The amount of the organic solvent is preferably 0.5 parts by mass or more and 50 parts by mass or less, more preferably 0.7 parts by mass or more and 20 parts by mass or less, and further preferably 1 part by mass or more and 10 parts by mass or less relative to sum of a mass of the base, and a mass of raw material compounds.

As the base, a base used in the Williamson's ether synthesis can be used with no particular limitation. Suitable examples of the base include sodium carbonate, potassium carbonate, cesium carbonate, sodium hydroxide, potassium hydroxide, lithium hydroxide, sodium hydride, potassium hydride, metallic sodium, and metallic potassium.

An amount of the base used in the reaction of the compound represented by the formula (A1-a), the compound represented by the formula (A1-b), and the compound represented by the formula A1-c) is not particularly limited as long as desired amount of the compound represented by the formula (A1-d) can be formed. For example, amount of the base is preferably 1.6 moles or more and 20 moles or less, more preferably 0.9 moles or more and 5 moles or less, and further preferably 1 mole or more and 3 moles or less relative to 1 mole of the compound represented by the formula (A1-a).

An amount of the compound represented by the formula (A1-b) and an amount of the compound represented by the formula (A1-c) is not particularly limited as long as desired amount of the compound represented by the formula (A1-d) can be formed. For example, amounts of the compound represented by the formula (A1-1b) and the compound represented by the formula (A1-c) are, respectively, preferably 0.8 moles or more and 10 moles or less, more preferably 0.9 moles or more and 5 moles or less, and further preferably 1 mole or more and 3 moles or less relative to 1 mole of the compound represented by the formula (A1-a).

A temperature for reacting the compound represented by the formula (A1-a) with the compound represented by the formula (A1-b) and the compound represented by the formula (A1-c) is not particularly limited as long as the compound represented by the formula (A1-d) in a desired amount can be formed. For example, the reaction temperature is preferably 0° C. or higher and 200° C. or lower, more preferably 10° C. or higher and 180° C. or lower, and further preferably 20° C. or higher and 150° C. or lower. In case of carrying out the reaction at a temperature higher than the boiling point of the organic solvent, the reaction is preferably carried out using a pressure-resistant vessel. Time for reacting the compound represented by the formula (A1-a) with the compound represented by the formula (A1-b) and the compound represented by the formula (A1-c) is not particularly limited as long as the compound represented by the formula (A1-d) in a desired amount can be formed. Typically, reaction time is preferably 1 hour or longer and 2 days or shorter, more preferably 2 hours or longer and 1 day or shorter, and further preferably 3 hours or longer and 18 hour or shorter.

Subsequently, hydrogen atom in the terminus represented by —X^{a03}—H is substituted with the group represented by Ran, and hydrogen atoms in the terminus represented by —X^{a4}—H is substituted with the group represented by R^{a03} in the compound represented by the formula (A1-d) obtained according to the above method. A method for substituting hydrogen atom in the terminus represented by —X^{a03}—H with the group represented by Ran is not particularly limited. The method for substituting hydrogen atom in the terminus represented by —X^{a03}—H with the group represented by Ran is appropriately selected according to type of the radically polymerizable group-containing group or the cationically polymerizable group-containing group represented by R^{a01}.

For example, when R^{a01} is (meth)acryloyl group, hydrogen atom in the terminus represented by —X^{a03}—H can be substituted with (meth)acryloyl group, for example, by reacting a (meth)acryloyl halide such as (meth)acryloyl chloride with the group represented by —X^{a3}—H in the compound represented by the formula (A1-1c). Reaction of the compound represented by the formula (A1-1c) with the (meth)acrylic acid halide is preferably carried out in an organic solvent. Type of the organic solvent is not particularly limited as long as the organic solvent does not react with the compound represented by the formula (A1-1c) and (meth)acrylic acid halide. Alternatively, the compound represented by the formula (A1-1) can also be obtained by condensing a (meth)acrylic acid and the compound represented by the formula (A1-c) according to known ester synthesis method.

When R^{a}n is glycidyl group, hydrogen atom in the terminus represented by —X^{a3}—H can be substituted with glycidyl group by reacting the group represented by —X^{a3}—H in the compound represented by the formula (A1-1c) with epichlorohydrin according to usual method. When R^{a}n is vinyl group, the group represented by —X^{a03}—H in the compound represented by the formula (A1-1c) can be directly vynilized with acetylene according to usual method.

A method of substituting hydrogen atom in a terminus represented by —X^{a04}—H with the group represented by R^{a03} is the same as the method of substituting hydrogen atom in the terminus represented by —X^{a03}—H with the group represented by R^{a01},

Alternatively, the compound represented by the formula (A1) can also be produced a method including obtaining the compound represented by the formula (A1) by reacting a compound represented by following formula (A1-1a), a compound represented by following formula (A1-e), and a compound represented by following formula (A1-f) in presence of a base.

H—X^{a01}-Ph¹-S-Ph²-X^{a02}—H (A1-a)

R^{a01}—X^{a03}—R^{a02}-Hal (A1-e)

R^{a03}—X^{a04}—R^{a04}-Hal (A1-f)

In the formula (A1-a), the formula (A1-e), and the formula (A1-f), R^{a01} to R^{a4}, X^{a01} to X^{a04}, Ph¹ and Ph² are the same as these in the formula (A1). Hal is a halogen atom.

The reaction of the compound represented by the formula (A1-a), the compound represented by the formula (A1-e), and the compound represented by the formula (A1-f) can be carried out in the same manner as the reaction of the compound represented by the formula (A1-a), the compound represented by the formula (A1-b), and the formula (A1-c).

The compound represented by the formula (A1) produced by the above-described method is purified as necessary and then added to the composition. Examples of a purification method include well-known methods such as a chromatography including a column chromatography and recrystallization.

The photopolymerizable compound (A) may include other photopolymerizable compound (A2) other than the compound (A1) described above as long as the desired effect is impaired. A ratio of a mass of the compound (A1) is preferably 50% by mass or more, more preferably 70% by mass or more, further preferably 80% by mass or more, particularly preferably 90% by mass, and most preferably 100% by mass relative to a mass of the photopolymerizable compound (A).

### [Other Photopolymerizable Compound (A2)]

As described above, the composition can include other photopolymerizable compound (A2) with the compound (A1). When the compound (A1) has the radically polymerizable group-containing group, other photopolymerizable compound (A2) also has the radically polymerizable group-containing group. When the compound (A1) has the cationically polymerizable group-containing group, other photopolymerizable compound (A2) also has the cationically polymerizable group-containing group.

When other photopolymerizable compound (A2) has radically polymerizable group-containing group(s), other photopolymerizable compound (A2) may be a monofunctional compound having one radically polymerizable group-containing group, or polyfunctional compound having two or more radically polymerizable group-containing group, and is preferably the polyfunctional compound. As other photopolymerizable compound (A2) having the radically polymerizable group-containing group, a compound having one or more (meth)acryloyl groups such as (meth)acrylate compound and (meth)acrylic amide compound is preferred, and the (meth)acrylate compound having one or more (meth)acryloyloxy groups is more preferred.

Examples of the monofunctional compound having the radically polymerizable group-containing group include (meth)acrylamide, methylol (meth)acrylamide, methoxymethyl (meth)acrylamide, ethoxymethyl (meth)acrylamide, propoxymethyl (meth)acrylamide, butoxymethoxymethyl (meth)acrylamide, N-methylol (meth)acrylamide, N-hydroxymethyl (meth)acrylamide, (meth)acrylic acid, fumaric acid, maleic acid, maleic anhydride, itaconic acid, itaconic anhydride, citraconic acid, citraconic anhydride, crotonic acid, 2-acrylamide-2-methylpropanesulfonic acid, tert-butylacrylamidesulfonic acid, methyl (meth)acrylate, ethyl (meth)acrylate, butyl (meth)acrylate, 2-ethylhexyl (meth)acrylate, cyclohexyl (meth)acrylate, 2-hydroxyethyl (meth)acrylate, 2-hydroxypropyl (meth)acrylate, 2-hydroxybutyl (meth)acrylate, 2-phenoxy-2-hydroxypropyl (meth)acrylate, 2-(meth)acryloyloxy-2-hydroxypropyl phthalate, glycerin mono(meth)acrylate, tetrahydrofurfuryl (meth)acrylate, dimethylamino (meth)acrylate, glycidyl (meth)acrylate, 2,2,2-trifluoroethyl (meth)acrylate, 2,2,3,3-tetrafluoropropyl (meth)acrylate, a half (meth)acrylate of phthalic acid derivatives, and the like. These monofunctional compounds may be used alone, or in combination of two or more types thereof.

Examples of the polyfunctional compound having the radically polymerizable group-containing group include ethylene glycol di(meth)acrylate, diethylene glycol di(meth)acrylate, tetraethylene glycol di(meth)acrylate, propylene glycol di(meth)acrylate, polypropylene glycol di(meth)acrylate, butylene glycol di(meth)acrylate, neopentyl glycol di(meth)acrylate, 1,6-hexane glycol di(meth)acrylate, trimethylolpropane tri(meth)acrylate, glycerin di(meth)acrylate, pentaerythritol triacrylate, pentaerythritol tetraacrylate, dipentaerythritol pentaacrylate, dipentaerythritol hexaacrylate, pentaerythritol di(meth)acrylate, pentaerythritol tri(meth)acrylate, pentaerythritol tetra(meth)acrylate, dipentaerythritol penta(meth)acrylate, dipentaerythritol hexa(meth)acrylate, 2,2-bis(4-(meth)acryloxydiethoxyphenyl)propane, 2,2-bis(4-(meth)acryloxypolyethoxyphenyl)propane, 2-hydroxy-3-(meth)acryloyloxypropyl (meth)acrylate, ethylene glycol diglycidyl ether di(meth)acrylate, diethylene glycol diglycidyl ether di(meth)acrylate, phthalic acid diglycidyl ester di(meth)acrylate, glycerin triacrylate, glycerin polyglycidyl ether poly(meth)acrylate, urethane (meth)acrylate (i.e., tolylene diisocyanate, trimethylhexamethylene diisocyanate, or a reaction product of hexamethylene diisocyanate and 2-hydroxyethyl (meth)acrylate), methylenebis(meth)acrylamide, (meth)acrylamide methylene ether, a polyfunctional compound such as a fused product of polyhydric alcohol and N-methylol(meth)acrylamide, triacryl formal, and the like. These polyfunctional compounds may be used alone, or in combination of two or more types thereof.

Among these other photopolymerizable compounds (A2) having the radically polymerizable group-containing group, a trifunctional or higher polyfunctional compound is preferred, a tetrafunctional or higher polyfunctional compound is more preferred, and a pentafunctional or higher polyfunctional compound is further preferred, in view of tendency to increase the strength of the cured product formed by using the composition.

The composition includes inorganic microparticles (B) described below. Depending on composition of the composition, localization of inorganic particles (B) may occur such that a layer rich in inorganic particles (B) and a layer poor in inorganic particles (B) are formed in the film formed by using the composition. From the viewpoint of suppressing such localization, the composition preferably includes a compound represented by following formula (A-2a) or a compound represented by following formula (A-2b) as photopolymerizable compound (A) having the radically polymerizable group-containing group.

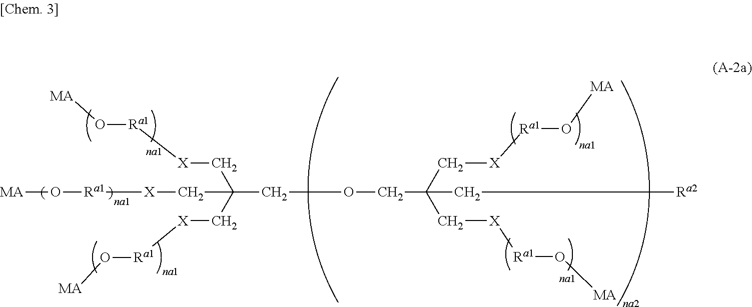

(MA-(O—R^{a1})ₙₐ₁—X—CH₂)₂—CH—X—(R^{a1}—O)ₙₐ₁MA (A-2b)

In the formula (A-2a) and the formula (A-2b), MAs are each independently a (meth)acryloyl group. Xs are each independently oxygen atom, —NH—, or —N(CH₃)—. R^{a1}s are each independently ethane-1,2-diyl group, propane-1,2-diyl group, or propane-1,3-diyl group. R^{a2} is a hydroxy group, an alkyl group having 1 or more and 4 or less carbon atoms, or a group represented by —X—(R^{a1}—O)ₙₐ₁-MA. X is the same as above. na1 and na2 are each independently 0 or 1.

In the formula (A-2a), examples of the alkyl group having 1 or more and 4 or less carbon atoms as R^{a2} include methyl group, ethyl group, n-propyl group, isopropyl group, n-butyl group, isobutyl group, sec-butyl group, and tert-butyl group. Among these alkyl groups, methyl group and ethyl group are preferred.

Suitable examples of the compound represented by the formula (A-2a), and the compound represented by the formula (A-2b) include pentaerythritol tetra(meth)acrylate, dipentaerythritol hexa(meth)acrylate, trimethylolpropane tri(meth)acrylate, glycerine tri(meth)acrylate, and the following compounds of 1) to 32). In following compounds of 1) to 32), MA is (meth)acryloyl group.
1) (MA-NH—CH₂)₄—C
2) (MA-N(CH₃)—CH₂)₄—C
3) (MA-O—CH₂CH₂CH₂—O—CH₂)₄—C
4) (MA-O—CH₂CH₂—O—CH₂)₄—C
5) (MA-O—CH₂CH₂CH₂—NH—CH₂)₄—C
6) (MA-O—CH₂CH₂—NH—CH₂)₄—C
7) (MA-O—CH₂CH₂CH₂—N(CH₃)—CH₂)₄—C
8) (MA-O—CH₂CH₂—N(CH₃)—CH₂)₄—C
9) (MA-NH—CH₂)₃—C—CH₂—O—CH₂—C—(CH₂—NH-MA) 3
10) (MA-N(CH₃)—CH₂)₃—C—CH₂—O—CH₂—C—(CH₂—N(CH₃)-MA)₃
11) (MA-O—CH₂CH₂CH₂—O—CH₂)₃—C—CH₂—O—CH₂—C—(CH₂—O—CH₂CH₂CH₂—O-MA)₃
12) (MA-O—CH₂CH₂—O—CH₂)₃—C—CH₂—O—CH₂—C—(CH₂—O—CH₂CH₂—O-MA)₃
13) (MA-O—CH₂CH₂CH₂—NH—CH₂)₃—C—CH₂—O—CH₂—C—(CH₂—NH—CH₂CH₂CH₂—O-MA)₃
14) (MA-O—CH₂CH₂—NH—CH₂)₃—C—CH₂—O—CH₂—C—(CH₂—NH—CH₂CH₂—O-MA)₃
15) (MA-O—CH₂CH₂CH₂—N(CH₃)—CH₂)₃—C—CH₂—O—CH₂—C—(CH₂—N(CH₃)—CH₂CH₂CH₂—O-MA)₃
16) (MA-O—CH₂CH₂—N(CH₃)—CH₂)₃—C—CH₂—O—CH₂—C—(CH₂—N(CH₃)—CH₂CH₂—O-MA)₃
17) (MA-NH—CH₂)₂—CH—NH-MA
18) (MA-N(CH₃)—CH₂)₂—CH—N(CH₃)-MA
19) (MA-O—CH₂CH₂CH₂—O—CH₂)₂—CH—O—CH₂CH₂CH₂—O-MA
20) (MA-O—CH₂CH₂—O—CH₂)₂—CH—CO—CH₂CH₂—O-MA
21) (MA-O—CH₂CH₂CH₂—NH—CH₂)₂—CH—NH—CH₂CH₂CH₂—O-MA
22) (MA-O—CH₂CH₂—NH—CH₂)₂—CH—NH—CH₂CH₂—O-MA
23) (MA-O—CH₂CH₂CH₂—N(CH₃)—CH₂)₂—CH—N(CH₃)—CH₂CH₂CH₂—O-MA
24) (MA-O—CH₂CH₂—N(CH₃)—CH₂)₂—CH—N(CH₃)—CH₂CH₂—O-MA
25) (MA-NH—CH₂)₃—C—CH₂CH₃
26) (MA-N(CH₃)—CH₂)₃—C—CH₂CH₃
27) (MA-O—CH₂CH₂CH₂—O—CH₂)₃—C—CH₂CH₃
28) (MA-O—CH₂CH₂—O—CH₂)₃—C—CH₂CH₃
29) (MA-O—CH₂CH₂CH₂—NH—CH₂)₃—C—CH₂CH₃
30) (MA-O—CH₂CH₂—NH—CH₂)₃—C—CH₂CH₃
31) (MA-O—CH₂CH₂CH₂—N(CH₃)—CH₂)₃—C—CH₂CH₃
32) (MA-O—CH₂CH₂—N(CH₃)—CH₂)₃—C—CH₂CH₃

From the viewpoint of suppressing localization of the inorganic microparticles (B) in a material formed by using the composition, a ratio of a sum of a mass of the compound represented by the formula (A-2a) and a sum of a mass of the compound represented by the formula (A-2b) is preferably 20% by mass or more and 50% by mass or less, more preferably 30% by mass or more and 50% by mass or less, and further preferably 40% by mass or more and 50% by mass or less relative to a mass of the photopolymerizable compound (A).

From the viewpoint of ease of forming a material with high refractive index by using the composition, the composition preferably includes a compound represented by following formula (A-2c) as other photopolymerizable compound (A2) having the radically polymerizable group-containing group.

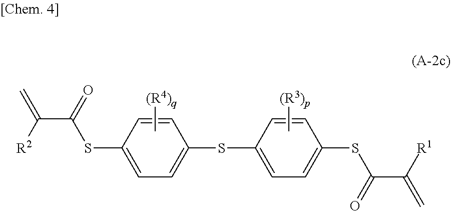

In the formula (A-2c), R¹ and R² are each independently hydrogen atom or methyl group. R³ and R⁴ are each independently an alkyl group having 1 or more and 5 or less carbon atoms.

p and q are each independently 0 or 1.

R¹ and R² are each independently hydrogen atom or methyl group. R¹ and R² may be different or may be the same. Since the compound represented by the formula (A-2c) is easily synthesized and available, R¹ and R² are preferably the same.

R³ and R⁴ are each independently an alkyl group having 1 or more and 5 or less carbon atoms. R³ and R⁴ may be different or may be the same. Since the compound represented by the formula (A-2c) is easily synthesized and available, R³ and R⁴ are preferably the same.

The alkyl group having 1 or more and 5 or less carbon atoms as R³ and R⁴ may be linear or branched. Examples of the alkyl group having 1 or more and 5 or less carbon atoms as R³ and R⁴ include methyl group, ethyl group, n-propyl group, isopropyl group, n-butyl group, isobutyl group, sec-butyl group, tert-butyl group, n-pentyl group, isopentyl group, and tert-pentyl group.

Suitable specific examples of the compound represented by the formula (A-2c) include following compounds.

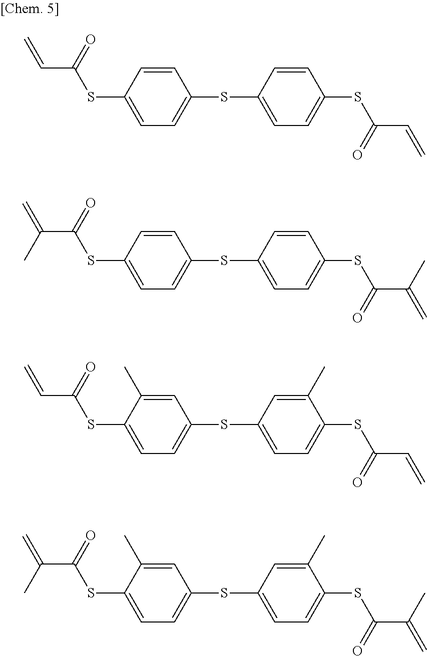

When the composition includes the compound represented by the formula (A-2c) as other photopolymerizable compound (A2) having the radically polymerizable group-containing group, a ratio of a mass of the compound represented by the formula (A-2c) is preferably 10% by mass or more and 50% by mass or less, and more preferably 30% by mass or more and 50% by mass or less relative to a mass of the photopolymerizable compound (A).

Since localization of the inorganic microparticles (B) in the material formed by using the composition is easily suppressed, the composition preferably includes a sulfur-containing (meth)acrylate represented by following formula (A-2d) as other photopolymerizable compound (A2) having the radically polymerizable group-containing group.

Ar^{a1}—R^{a21}—S—R^{a22}—O—CO—CR^{a23}=CH₂ (A-2)

In the formula (A-2d), Ar^{a1} is phenyl group optionally substituted with halogen atom. R^{a21} is single bond, or an alkylene group having 1 or more and 6 or less carbon atoms. R^{a22} is an alkylene group having 1 or more and 6 or less carbon atoms. R^{a23} is hydrogen atom or methyl group.

Ar^{a1} is the phenyl group optionally substituted with the halogen atom. When the phenyl group is substituted with the halogen atom, a number of halogen atoms bonding to the phenyl group is not particularly limited. A number of halogen atoms bonding to the phenyl group is preferably 1 or 2, and more preferably 1. When two or more halogen atoms bond to the phenyl group, a plurality of halogen atoms bonding to the phenyl group may consist of only halogen atoms of the same species or halogen atoms of two or more species. As the halogen atom which may bond to the phenyl group, fluorine atom, chlorine atom, bromine atom, and iodine atom are exemplified, and fluorine atom, chlorine atom, and bromine atom are preferred. Unsubstituted phenyl group is preferred as Aral.

R^{a21} is a single bond or an alkylene group having 1 or more and 6 or less carbon atoms. Examples of the alkylene group having 1 or more and 6 or less carbon atoms include methylene group, ethane-1,2-diyl group, propane-1,2-diyl group, propane-1,3-diyl group, butane-1,4-diyl group, pentane-1,5-diyl group, and hexane-1,6-diyl group. R^{a21} is preferably single bond or methylene group, and more preferably single bond.

R^{a22} is an alkylene group having 1 or more and 6 or less carbon atoms. Examples of the alkylene group having 1 or more and 6 or less carbon atoms include methylene group, ethane-1,2-diyl group, propane-1,2-diyl group, propane-1,3-diyl group, butane-1,4-diyl group, pentane-1,5-diyl group, and hexane-1,6-diyl group.

In view of easy availability of the sulfur-containing (meth)acrylate, and suppressing localization of the inorganic microparticles (B) in a material formed by using the composition, it is particularly preferred that Ar^{a1} is phenyl group and R^{a2}1 is single bond.

Specific examples of the sulfur-containing (meth)acrylate represented by the formula (A-2d) include 2-phenylthioethyl (meth)acrylate, 3-phenylthiopropyl (meth)acrylate, 2-benzylthioethyl (meth)acrylate, 3-benzylthiopropyl (meth)acrylate, 2-(2-chlorophenyl)ethyl (meth)acrylate, 2-(3-chlorophenyl)ethyl (meth)acrylate, 2-(4-chlorophenyl)ethyl (meth)acrylate, 3-(2-chlorophenyl)propyl (meth)acrylate, 3-(3-chlorophenyl)propyl (meth)acrylate, 3-(4-chlorophenyl)propyl (meth)acrylate, 2-(2-fluorophenyl)ethyl (meth)acrylate, 2-(3-fluorophenyl)ethyl (meth)acrylate, 2-(4-fluorophenyl)ethyl (meth)acrylate, 3-(2-fluorophenyl)propyl (meth)acrylate, 3-(3-fluorophenyl)propyl (meth)acrylate, 3-(4-fluorophenyl)propyl (meth)acrylate, 2-(2-bromophenyl)ethyl (meth)acrylate, 2-(3-bromophenyl)ethyl (meth)acrylate, 2-(4-bromophenyl)ethyl (meth)acrylate, 3-(2-bromophenyl)propyl (meth)acrylate, 3-(3-bromophenyl)propyl (meth)acrylate, and 3-(4-bromophenyl)propyl (meth)acrylate.

When the composition includes the sulfur-containing (meth)acrylate represented by the formula (A-2d) as other photopolymerizable compound, a ratio of a mass of the sulfur-containing (meth)acrylate represented by the formula (A-2d) is preferably 40% by mass or more and 50% by mass or less relative to the mass of the photopolymerizable compound (A).

When the compound (A1) has the cationically polymerizable group, other photopolymerizable compound (A2) may be a monofunctional compound having one cationically polymerizable group, or a polyfunctional compound having two or more cationically polymerizable group, and is preferably the polyfunctional compound.

When the compound (A1) has the vinyloxy group as the cationically polymerizable group-containing group, the composition may include a vinyl ether compound as other photopolymerizable compound (A2). Such vinyl ether compound may be a monofunctional compound or may be a polyfunctional compound.

Suitable specific examples of the vinyl ether compound include aromatic monovinyl ether compounds such as viny phenyl ether, 4-vinyloxytoluene, 3-vinyloxytoluene, 2-vinyloxytoluene, 1-vinyloxy-4-chlorobenzene, 1-vinyloxy-3-chlorobenzene, 1-vinyloxy-2-chlorobenzene, 1-vinyloxy-2,3-dimethylbenzene, 1-vinyloxy-2,4-dimethylbenzene, 1-vinyloxy-2,5-dimethylbenzene, 1-vinyloxy-2,6-dimethylbenzene, 1-vinyloxy-3,4-dimethylbenzene, 1-vinyloxy-3,5-dimethylbenzene, 1-vinyloxynaphthalene, 2-vinyloxynaphthalene, 2-vinyloxyfluorene, 3-vinyloxyfluorene, 4-vinyloxy-1,1′-biphenyl, 3-vinyloxy-1,1′-biphenyl, 2-vinyloxy-1,1′-biphenyl, 6-vinyloxytetralin, and 5-vinyloxytetralin; aromatic divinyl ether compounds such as 1,4-divinyloxybenzene, 1,3-divinyloxybenzene, 1,2-divinyloxybenzene, 1,4-divinyloxynaphthalene, 1,3-divinyloxynaphthalene, 1,2-divinyloxynaphthalene, 1,5-divinyloxynaphthalene, 1,6-divinyloxynaphthalene, 1,7-divinyloxynaphthalene, 1,8-divinyloxynaphthalene, 2,3-divinyloxynaphthalene, 2,6-divinyloxynaphthalene, 2,7-divinyloxynaphthalene, 1,2-divinyloxyfluorene, 3,4-divinyloxyfluorene, 2,7-divinyloxyfluorene, 4,4′-divinyloxybiphenyl, 3,3′-divinyloxybiphenyl, 2,2′-divinyloxybiphenyl, 3,4′-divinyloxybiphenyl, 2,3′-divinyloxybiphenyl, 2,4′-divinyloxybiphenyl, and bisphenol A divinyl ether. These vinyl ether compounds can be used in combination of two or more types thereof.

When the compound (A1) has the epoxy group-containing group as the cationically polymerizable group-containing group, the composition may include various epoxy compounds as other photopolymerizable compound (A2). Examples of the epoxy compound include difunctional epoxy resins such as bisphenol A type epoxy resin, bisphenol F type epoxy resin, bisphenol S type epoxy resin, bisphenol AD type epoxy resin, naphthalene type epoxy resin, and biphenyl type epoxy resin; novolak epoxy resins such as phenol novolak type epoxy resin, brominated phenol novolak type epoxy resin, ortho-cresol novolak type epoxy resin, bisphenol A novolak type epoxy resin, and bisphenol AD novolak type epoxy resin; cyclic aliphatic epoxy resins such as epoxidized product of a dicyclopentadiene type phenol resin; aromatic epoxy resins such as epoxidized product of a naphthalene type phenol resin; glycidyl ester type epoxy resins such as dimer acid glycidyl ester and triglycidyl ester; glycidylamine type epoxy resins such as tetraglycidylaminodiphenylmethane, triglycidyl-p-aminophenol, tetraglycidylmetaxylylenediamine, and tetraglycidyl bisaminomethylcyclohexane; heterocyclic epoxy resins such as triglycidyl isocyanurate; trifunctional epoxy resins such as phloroglucinol triglycidyl ether, trihydroxybiphenyl triglycidyl ether, trihydroxyphenylmethane triglycidyl ether, glycerin triglycidyl ether, 2-[4-(2,3-epoxypropoxy)phenyl]-2-[4-[1,1-bis[4-(2,3-epoxypropoxy)phenyl]ethyl]phenyl]propane, and 1,3-bis[4-[1-[4-(2,3-epoxypropoxy)phenyl]-1-[4-[1-[4-(2,3-epoxypropoxy)phenyl]-1-methylethyl]phenyl]ethyl]phenoxy]-2-propanol; tetrafunctional epoxy resins such as tetrahydroxyphenylethane tetraglycidyl ether, tetraglycidylbenzophenone, bisresorcinol tetraglycidyl ether, and tetraglycidoxybiphenyl; and a 1,2-epoxy-4-(2-oxyranyl)cyclohexane adduct of 2,2-bis(hydroxymethyl)-1-butanol. The 1,2-epoxy-4-(2-oxiranyl)cyclohexene adduct of 2,2-bis(hydroxymethyl)-1 butanol is commercially available as EHPE-3150 (manufactured by Daicel Corporation).

In addition, the oligomer or polymer type polyfunctional epoxy compound may also be suitably used. Typical examples of the oligomer or polymer type polyfunctional epoxy compound include a phenol novolak type epoxy compound, a brominated phenol novolak type epoxy compound, an ortho-cresol novolak type epoxy compound, a xylenol novolak type epoxy compound, a naphthol novolak type epoxy compound, a bisphenol A novolak type epoxy compound, a bisphenol AD novolak type epoxy compound, an epoxidized product of a dicyclopentadiene type phenol resin, an epoxidized product of a naphthalene type phenol resin, and the like.

Other examples of the suitable epoxy compound include a polyfunctional alicyclic epoxy compound having an alicyclic epoxy group.

Specific examples of the alicyclic epoxy compound include 2-(3,4-epoxycyclohexyl-5,5-spiro-3,4-epoxy)cyclohexane-meta-dioxane, bis(3,4-epoxycyclohexylmethyl)adipate, bis(3,4-epoxy-6-methylcyclohexylmethyl)adipate, 3,4-epoxy-6-methylcyclohexyl-3′,4′-epoxy-6′-methylcyclohexanecarboxylate, s-caprolactone-modified 3,4-epoxycyclohexylmethyl-3′,4′-epoxycyclohexanecarboxylate, trimethylcaprolactone-modified 3,4-epoxycyclohexylmethyl-3′,4′-epoxycyclohexanecarboxylate, β-methyl-5-valerolactone-modified 3,4-epoxycyclohexylmethyl-3′,4′-epoxycyclohexane carboxylate, methylenebis(3,4-epoxycyclohexane), di(3,4-epoxycyclohexylmethyl)ether of ethylene glycol, ethylenebis(3,4-epoxycyclohexane carboxylate), epoxycyclohexahydrophthalate, di-2-ethylhexyl epoxycyclohexahydrophthalate, and an epoxy resin having a tricyclodecene oxide group and compounds represented by the following formulas (a01-1) to (a01-5).

Among these specific examples of the alicyclic epoxy compound, alicyclic epoxy compounds represented by the following formulas (a01-1) to (a01-5) are preferred, since the cured product with high hardness can be formed.

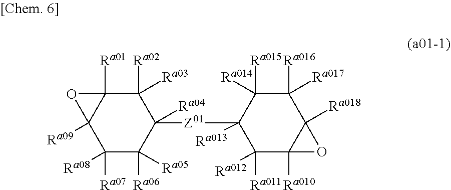

In the formula (a01-1), Z⁰¹ represents a single bond or a linking group (divalent group having one or more atoms). R^{a01} to R^{a018} each independently represent a group selected from the group consisting of a hydrogen atom, a halogen atom, and an organic group.

Examples of the linking group Z⁰¹ include a divalent hydrocarbon group, a divalent group selected from the group consisting of —O—, —O—CO—, —S—, —SO—, —SO₂—, —CBr₂—, —C(CBr₃)₂—, —C(CF₃)₂—, and —R^{a019}—O—CO—, and a group in which plural these groups are bonded.

Examples of the divalent hydrocarbon group as the linking group Z⁰¹ include a linear or branched alkylene group having 1 or more and 18 or less carbon atoms, a divalent alicyclic hydrocarbon group, and the like. Examples of the linear or branched alkylene group having 1 or more and 18 or less carbon atoms include methylene group, methylmethylene group, dimethylmethylene group, dimethylene group, trimethylene group, and the like. Examples of the divalent alicyclic hydrocarbon group include cycloalkylene groups (including a cycloalkylidene group) such as 1,2-cyclopentylene group, 1,3-cyclopentylene group, cyclopentylidene group, 1,2-cyclohexylene group, 1,3-cyclohexylene group, 1,4-cyclohexylene group, cyclohexylidene group, and the like.

R^{a019} is an alkylene group having 1 or more and 8 or less carbon atoms, and preferably a methylene group or an ethylene group.

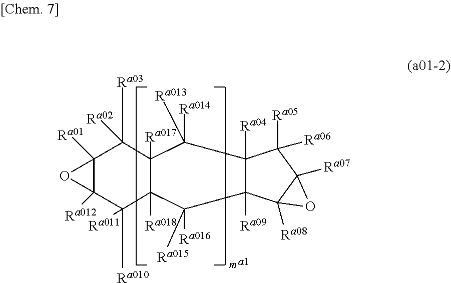

In the formula (a01-2), R^{a01} to R^{a018} are groups selected from the group consisting of hydrogen atom, a halogen atom, and an organic group. R^{a02} and R^{a010} may be combined with each other. R^{a013} and R^{a016} may be combined with each other to form a ring. m^{a1} is 0 or 1.

As the alicyclic epoxy compound represented by the formula (a01-2), a compound represented by the formula (a01-2-1) that corresponds to a compound in which m^{a1} in the formula (a01-2) is 0 is preferred.

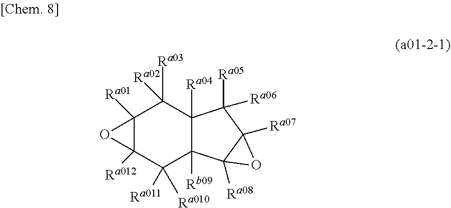

In the formula (a01-2-1), R^{a01} to R^{a012} are groups selected from the group consisting of a hydrogen atom, a halogen atom, and an organic group. R^{a02} and R^{a010} may be combined with each other to form a ring.

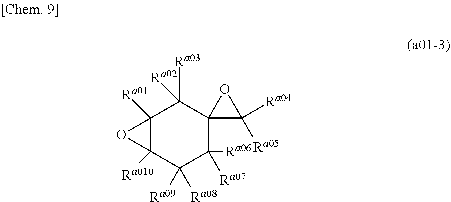

In the formula (a01-3), R^{a01} to R^{a010} are groups selected from the group consisting of a hydrogen atom, a halogen atom, and an organic group. R^{a02} and R^{a08} may be combined with each other.

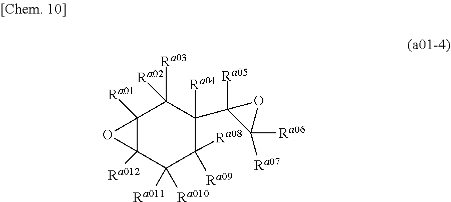

In the formula (a01-4), R^{a01} to R^{a012} are groups selected from the group consisting of a hydrogen atom, a halogen atom, and an organic group. R^{a02} and R^{a010} may be combined with each other.

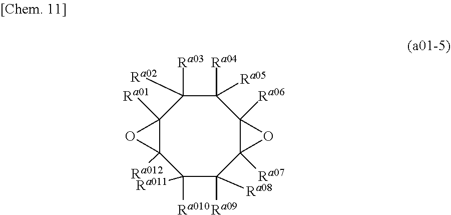

In the formula (a01-5), R^{a01} to R^{a012} are groups selected from the group consisting of hydrogen atom, a halogen atom, and an organic group.

In the formulas (a01-1) to (a01-5), when R^{a01} to R^{a018} are organic groups, the organic group is not particularly limited as long as the object of the present invention is not impaired, and may be a hydrocarbon group, a group composed of a carbon atom and a halogen atom, or a group containing heteroatoms such as a halogen atom, an oxygen atom, a sulfur atom, a nitrogen atom, and a silicon atom, together with a carbon atom and a hydrogen atom. Examples of the halogen atom include chlorine atom, bromine atom, iodine atom, and fluorine atom.

The organic group is preferably a hydrocarbon group, a group composed of carbon atom, hydrogen atom, and oxygen atom, a halogenated hydrocarbon group, a group composed of carbon atom, oxygen atom, and a halogen atom, and a group composed of carbon atom, hydrogen atom, oxygen atom, and a halogen atom. When the organic group is the hydrocarbon group, the hydrocarbon group may be an aromatic hydrocarbon group, an aliphatic hydrocarbon group, or a group including an aromatic skeleton and an aliphatic skeleton. The number of carbon atoms in the organic group is preferably 1 or more and 20 or less, more preferably 1 or more and 10 or less, and particularly preferably 1 or more and 5 or less.

Specific examples of the hydrocarbon group include chain alkyl groups such as methyl group, ethyl group, n-propyl group, isopropyl group, n-butyl group, isobutyl group, sec-butyl group, tert-butyl group, n-pentyl group, n-hexyl group, n-heptyl group, n-octyl group, 2-ethylhexyl group, n-nonyl group, n-decyl group, n-undecyl group, n-tridecyl group, n-tetradecyl group, n-pentadecyl group, n-hexadecyl group, n-heptadecyl group, n-octadecyl group, n-nonadecyl group, and n-icosyl group; chain alkenyl groups such as vinyl group, 1-propenyl group, 2-n-propenyl group (allyl group), 1-n-butenyl group, 2-n-butenyl group, and 3-n-butenyl group; cycloalkyl groups such as cyclopropyl group, cyclobutyl group, cyclopentyl group, cyclohexyl group, and cycloheptyl group; aryl groups such as phenyl group, o-tolyl group, m-tolyl group, p-tolyl group, α-naphthyl group, β-naphthyl group, biphenyl-4-yl group, biphenyl-3-yl group, biphenyl-2-yl group, anthryl group, and phenanthryl group; and aralkyl groups such as benzyl group, phenethyl group, α-naphthylmethyl group, β-naphthylmethyl group, α-naphthylethyl group, and β-naphthylethyl group.

Specific examples of the halogenated hydrocarbon group include halogenated chain alkyl groups such as chloromethyl group, dichloromethyl group, trichloromethyl group, bromomethyl group, dibromomethyl group, tribromomethyl group, fluoromethyl group, difluoromethyl group, trifluoromethyl group, 2,2,2-trifluoroethyl group, pentafluoroethyl group, heptafluoropropyl group, perfluorobutyl group, perfluoropentyl group, perfluorohexyl group, perfluoroheptyl group, perfluorooctyl group, perfluorononyl group, and perfluorodecyl group; halogenated cycloalkyl groups such as 2-chlorocyclohexyl group, 3-chlorocyclohexyl group, 4-chlorocyclohexyl group, 2,4-dichlorocyclohexyl group, 2-bromocyclohexyl group, 3-bromocyclohexyl group, and 4-bromocyclohexyl group; halogenated aryl groups such as 2-chlorophenyl group, 3-chlorophenyl group, 4-chlorophenyl group, 2,3-dichlorophenyl group, 2,4-dichlorophenyl group, 2,5-dichlorophenyl group, 2,6-dichlorophenyl group, 3,4-dichlorophenyl group, 3,5-dichlorophenyl group, 2-bromophenyl group, 3-bromophenyl group, 4-bromophenyl group, 2-fluorophenyl group, 3-fluorophenyl group, and 4-fluorophenyl group; and halogenated aralkyl groups such as 2-chlorophenylmethyl group, 3-chlorophenylmethyl group, 4-chlorophenylmethyl group, 2-bromophenylmethyl group, 3-bromophenylmethyl group, 4-bromophenylmethyl group, 2-fluorophenylmethyl group, 3-fluorophenylmethyl group, and 4-fluorophenylmethyl group.

Specific examples of the group composed of carbon atom, hydrogen atom, and oxygen atom include hydroxy chain alkyl groups such as hydroxymethyl group, 2-hydroxyethyl group, 3-hydroxy-n-propyl group, and 4-hydroxy-n-butyl group; halogenated cycloalkyl groups such as 2-hydroxycyclohexyl group, 3-hydroxycyclohexyl group, and 4-hydroxycyclohexyl group; hydroxyaryl groups such as 2-hydroxyphenyl group, 3-hydroxyphenyl group, 4-hydroxyphenyl group, 2,3-dihydroxyphenyl group, 2,4-dihydroxyphenyl group, 2,5-dihydroxyphenyl group, 2,6-dihydroxyphenyl group, 3,4-dihydroxyphenyl group, and 3,5-dihydroxyphenyl group; hydroxyaralkyl groups such as 2-hydroxyphenylmethyl group, 3-hydroxyphenylmethyl group, and 4-hydroxyphenylmethyl group; chain alkoxy groups such as methoxy group, ethoxy group, n-propoxy group, isopropoxy group, n-butyloxy group, isobutyloxy group, sec-butyloxy group, tert-butyloxy group, n-pentyloxy group, n-hexyloxy group, n-heptyloxy group, n-octyloxy group, 2-ethylhexyloxy group, n-nonyloxy group, n-decyloxy group, n-undecyloxy group, n-tridecyloxy group, n-tetradecyloxy group, n-pentadecyloxy group, n-hexadecyloxy group, n-heptadecyloxy group, n-octadecyloxy group, n-nonadecyloxy group, and n-icosyloxy group; chain alkenyloxy groups such as vinyloxy group, 1-propenyloxy group, 2-n-propenyloxy group (allyloxy group), 1-n-butenyloxy group, 2-n-butenyloxy group, and 3-n-butenyloxy group; aryloxy groups such as phenoxy group, o-tolyloxy group, m-tolyloxy group, p-tolyloxy group, α-naphthyloxy group, β-naphthyloxy group, biphenyl-4-yloxy group, biphenyl-3-yloxy group, biphenyl-2-yloxy group, anthryloxy group, and phenanthryloxy group; aralkyloxy groups such as benzyloxy group, phenethyloxy group, α-naphthylmethyloxy group, β-naphthylmethyloxy group, α-naphthylethyloxy group, and β-naphthylethyloxy group; alkoxyalkyl groups such as methoxymethyl group, ethoxymethyl group, n-propoxymethyl group, 2-methoxyethyl group, 2-ethoxyethyl group, 2-n-propoxyethyl group, 3-methoxy-n-propyl group, 3-ethoxy-n-propyl group, 3-n-propoxy-n-propyl group, 4-methoxy-n-butyl group, 4-ethoxy-n-butyl group, and 4-n-propoxy-n-butyl group; alkoxyalkoxy groups such as a methoxymethoxy group, ethoxymethoxy group, n-propoxymethoxy group, 2-methoxyethoxy group, 2-ethoxyethoxy group, 2-n-propoxyethoxy group, 3-methoxy-n-propoxy group, 3-ethoxy-n-propoxy group, 3-n-propoxy-n-propoxy group, 4-methoxy-n-butyloxy group, 4-ethoxy-n-butyloxy group, and 4-n-propoxy-n-butyloxy group; alkoxyaryl groups such as 2-methoxyphenyl group, 3-methoxyphenyl group, and 4-methoxyphenyl group; alkoxyaryloxy groups such as 2-methoxyphenoxy group, 3-methoxyphenoxy group, and 4-methoxyphenoxy group; aliphatic acyl groups such as formyl group, an acetyl group, propionyl group, butanoyl group, pentanoyl group, hexanoyl group, heptanoyl group, octanoyl group, nonanoyl group, and decanoyl group; aromatic acyl groups such as benzoyl group, α-naphthoyl group, and β-naphthoyl group; chain alkyloxycarbonyl groups such as methoxycarbonyl group, ethoxycarbonyl group, n-propoxycarbonyl group, n-butyloxycarbonyl group, n-pentyloxycarbonyl group, n-hexylcarbonyl group, n-heptyloxycarbonyl group, n-octyloxycarbonyl group, n-nonyloxycarbonyl group, and n-decyloxycarbonyl group; aryloxycarbonyl groups such as phenoxycarbonyl group, α-naphthoxycarbonyl group, and β-naphthoxycarbonyl group; aliphatic acyloxy groups such as formyloxy group, acetyloxy group, propionyloxy group, butanoyloxy group, pentanoyloxy group, hexanoyloxy group, heptanoyloxy group, octanoyloxy group, nonanoyloxy group, and decanoyloxy group; and aromatic acyloxy groups such as benzoyloxy group, α-naphthoyl oxy group, and β-naphthoyl oxy group.

R^{a01} to R^{a018} are each independently preferably a group selected from the group consisting of hydrogen atom, a halogen atom, an alkyl group having 1 or more and 5 or less carbon atoms, and an alkoxy group having 1 or more and 5 or less carbon atoms. All of R^{a01} to R^{a018} are more preferably hydrogen atoms since it is easy to form a material having particularly excellent mechanical properties by using the composition.

In the formulas (a01-2) to (a01-5), R^{a01} to R^{a018} are the same as R^{a01} to R^{a018} in the formula (a01-1). When R^{a02} and R^{a010} are combined with each other in the formula (a01-2) and the formula (a01-4), when R^{a013} and R^{a016} are combined with each other in the formula (a01-2), and when R^{a02} and R^{a08} are combined with each other in the formula (a01-3), for example, —CH₂— and —C(CH₃)₂— are exemplified as the divalent group to be formed.

Among the alicyclic epoxy compounds represented by the formula (a01-1), specific examples of suitable compound include alicyclic epoxy compounds represented by following formula (a01-1a), formula (a01-1b), and formula (a01-1c), 2,2-bis(3,4-epoxycyclohexan-1-yl)propane[=2,2-bis(3,4-epoxycyclohexyl)propane], and the like.

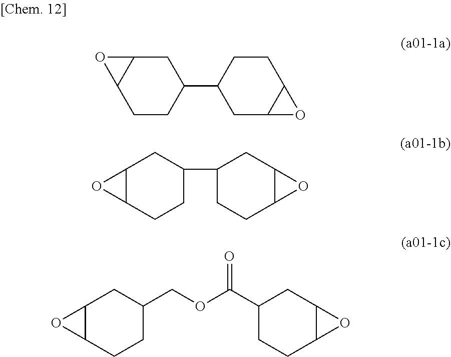

Among the alicyclic epoxy compounds represented by the formula (a01-2), specific examples of suitable compound include alicyclic epoxy compounds represented by following formulas (a01-2a) and (a01-2b).

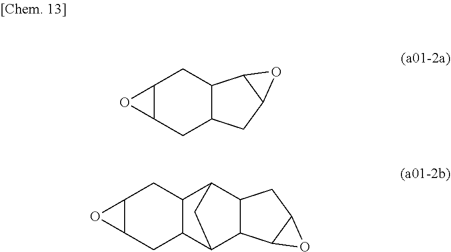 [SMILES: C1C2CC3OC3C2CC2OC12 ; C1C2OC2CC2C1C1CC2C2C1CC1OC12]

Among the alicyclic epoxy compounds represented by the formula (a01-3), specific examples of suitable compound include S spiro[3-oxatricyclo[3.2.1.0^{2,4}]octane-6,2′-oxirane], and the like.

Among the alicyclic epoxy compounds represented by the formula (a01-4), specific examples of suitable compound include 4-vinylcyclohexene dioxide, dipentene dioxide, limonene dioxide, 1-methyl-4-(3-methyloxiran-2-yl)-7-oxabicyclo[4.1.0]heptane, and the like.

Among the alicyclic epoxy compounds represented by the formula (a01-5), specific examples of suitable compound include 1,2,5,6-diepoxycyclooctane, and the like.

It is also possible to suitably use, as the epoxy compound, a compound represented by the following formula (a1-I).

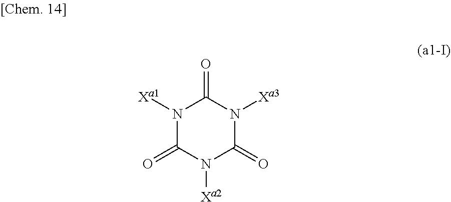

In the formula (a1-I), X^{a1}, X^{a2}, and X^{a3} each independently represent hydrogen atom, or an organic group optionally having an epoxy group, and the total number of epoxy groups possessed by X^{a1}, X^{a2}, and X^{a3} is 2 or more.

The compound represented by the formula (a1-I) is preferably a compound represented by following formula (a1-II).

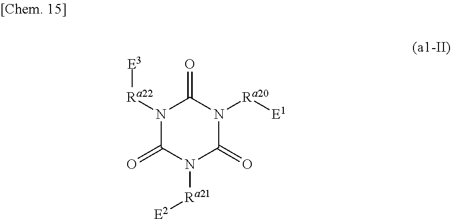

In the formula (a1-II), R^{a20} to R^{a22} represent a linear, branched, or cyclic alkylene group, an arylene group, —O—, —C(═O)—, —NH—, and a combination thereof, and each may be the same or different. E¹ to E³ represent at least one substituent or hydrogen atom selected from the group consisting of an epoxy group, an oxetanyl group, an ethylenically unsaturated group, an alkoxysilyl group, an isocyanate group, a blocked isocyanate group, a thiol group, a carboxy group, a hydroxy group, and a succinic anhydride group. Provided that, a total number of epoxy groups possessed by E¹, E², and E³ is 2 or more.

In the formula (a1-II), each of at least two of a group represented by R^{a20} and E¹, R^{a21} and E², and R^{a22} and E³ is preferably groups represented by the following formula (b1-IIa), and all the groups are more preferably groups represented by following formula (a1-IIa). Plural groups represented by the formula (a1-IIa) bonded to one compound are preferably the same groups

-L-C^{a} (a1-IIa)

In the formula (a1-IIa), L is a linear, branched, or cyclic alkylene group, an arylene group, —O—, —C(═O)—, —NH—, and a combination thereof. C^{a} is an oxyranyl group (epoxy group). In the formula (a1-IIa), L and C^{a} may be combined to form a cyclic structure.

In the formula (a1-IIa), the linear, branched, or cyclic alkylene group as L is preferably an alkylene group having 1 or more and 10 or less carbon atoms, and the arylene group as L is preferably an arylene group having 5 or more and 10 or less carbon atoms. In the formula (a1-IIa), L is preferably a linear alkylene group having 1 or more and 3 or less carbon atoms, a phenylene group, —O—, —C(═O)—, —NH—, and a combination thereof, and preferably at least one of a linear alkylene group having 1 or more and 3 or less carbon atoms such as a methylene group, and a phenylene group, or a group composed of a combination of these groups and at least one of —O—, —C(═O)— and —NH—.

In the formula (a1-IIa), when L and C^{a} are combined with each other to form a cyclic structure, for example, when a branched alkylene group and an epoxy group are combined with each other to form a cyclic structure (structure having an alicyclic structure epoxy group), organic groups represented by the following formulas (a1-IIb) to (a1-IId) are exemplified.

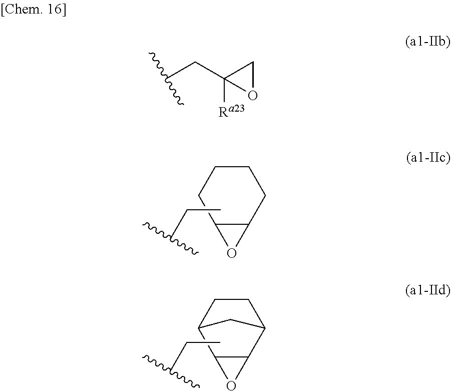

In the formula (a1-IIb), R^{a23} is hydrogen atom or methyl group.

Examples of the compound represented by the formula (a1-II) include, but are not limited to, examples of an epoxy compound having an oxyranyl group or an alicyclic epoxy group.

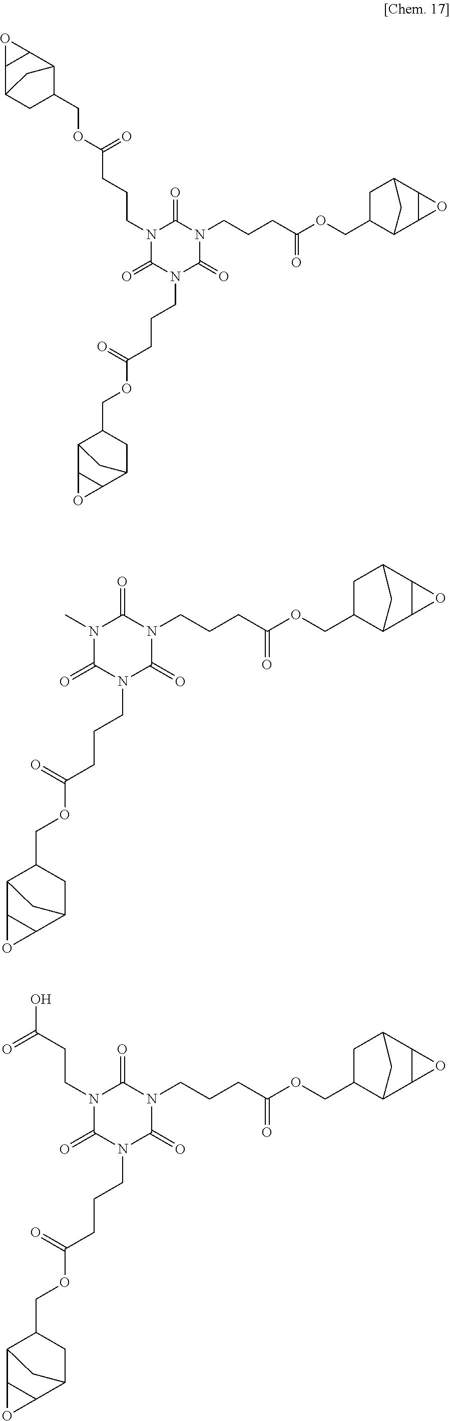 [SMILES: O=C(CCCn1c(=O)n(CCCC(=O)OCC2CC3CC2C2OC32)c(=O)n(CCCC(=O)OCC2CC3CC2C2OC32)c1=O)OCC1CC2CC1C1OC21 ; Cn1c(=O)n(CCCC(=O)OCC2CC3CC2C2OC32)c(=O)n(CCCC(=O)OCC2CC3CC2C2OC32)c1=O ; O=C(O)CCn1c(=O)n(CCCC(=O)OCC2CC3CC2C2OC32)c(=O)n(CCCC(=O)OCC2CC3CC2C2OC32)c1=O]

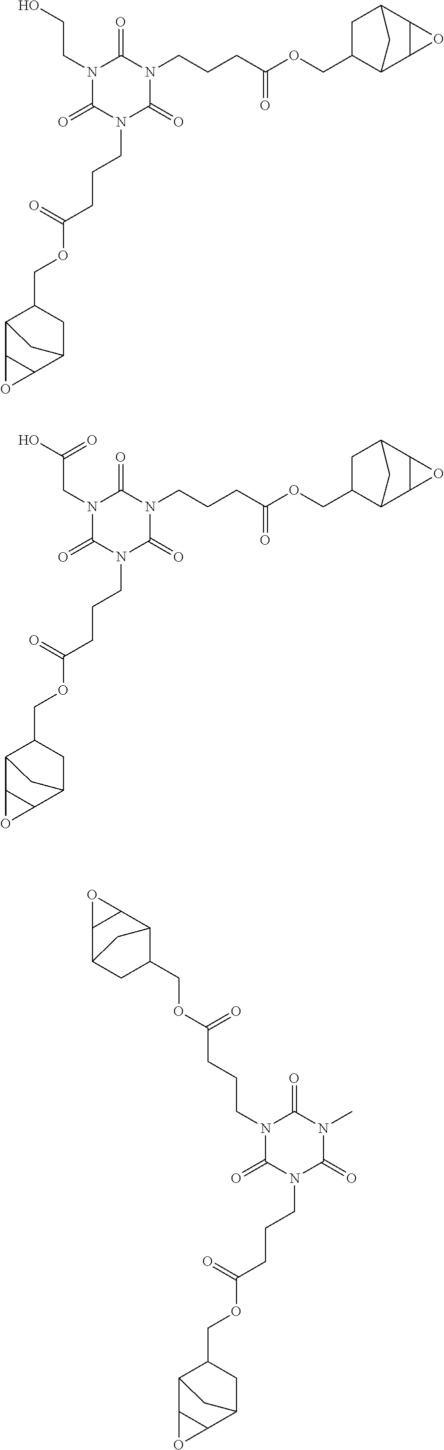 [SMILES: O=C(CCCn1c(=O)n(CCO)c(=O)n(CCCC(=O)OCC2CC3CC2C2OC32)c1=O)OCC1CC2CC1C1OC21 ; O=C(O)Cn1c(=O)n(CCCC(=O)OCC2CC3CC2C2OC32)c(=O)n(CCCC(=O)OCC2CC3CC2C2OC32)c1=O ; Cn1c(=O)n(CCCC(=O)OCC2CC3CC2C2OC32)c(=O)n(CCCC(=O)OCC2CC3CC2C2OC32)c1=O]

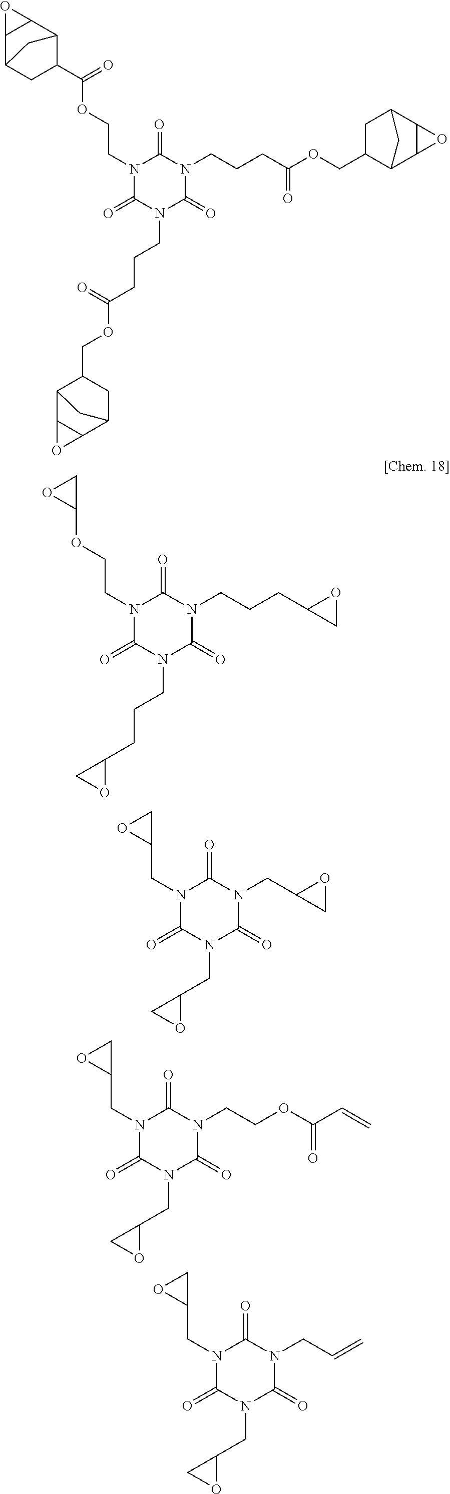

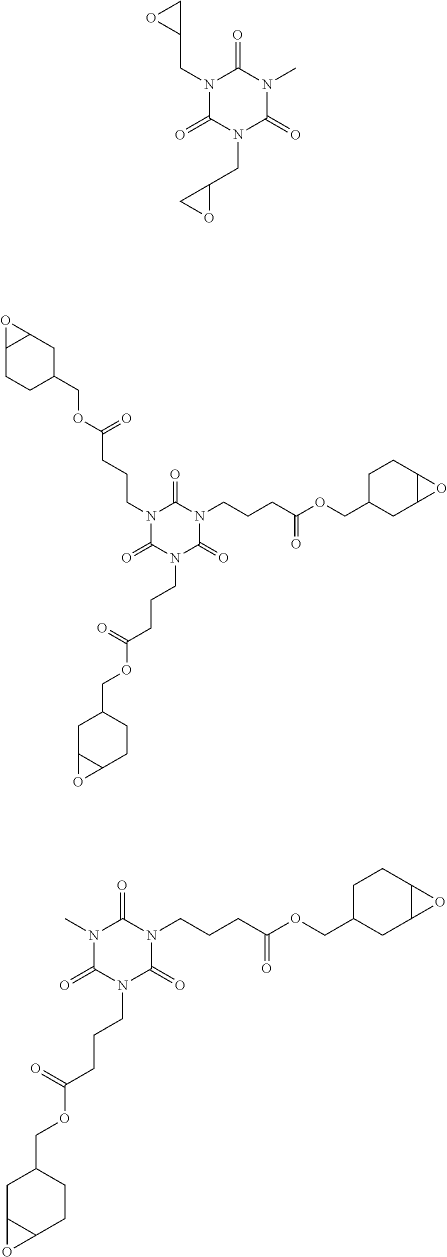 [SMILES: Cn1c(=O)n(CC2CO2)c(=O)n(CC2CO2)c1=O ; O=C(CCCn1c(=O)n(CCCC(=O)OCC2CCC3OC3C2)c(=O)n(CCCC(=O)OCC2CCC3OC3C2)c1=O)OCC1CCC2OC2C1 ; Cn1c(=O)n(CCCC(=O)OCC2CCC3OC3C2)c(=O)n(CCCC(=O)OCC2CCC3OC3C2)c1=O]

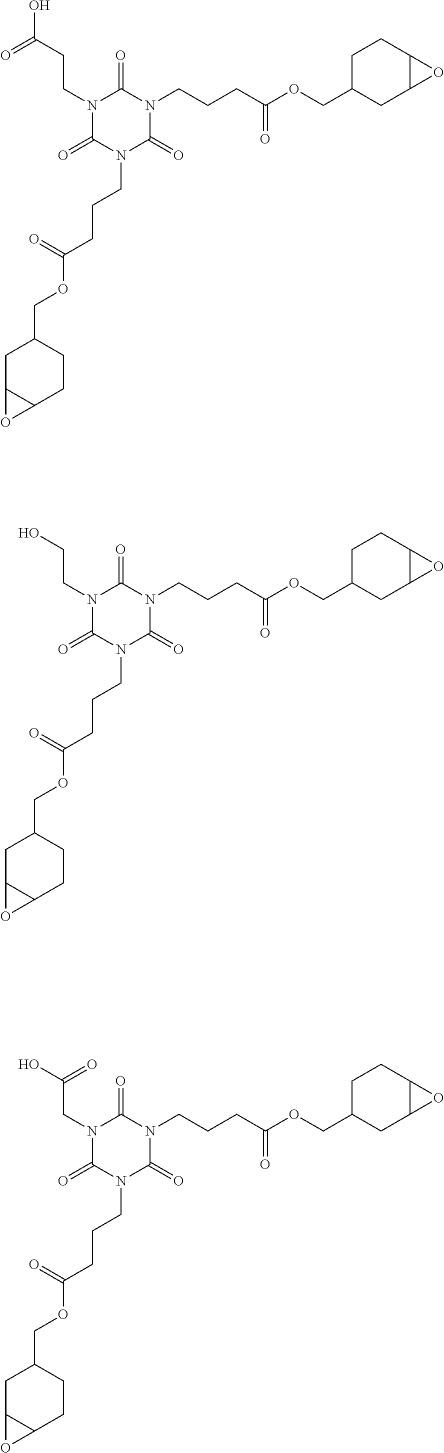 [SMILES: O=C(O)CCn1c(=O)n(CCCC(=O)OCC2CCC3OC3C2)c(=O)n(CCCC(=O)OCC2CCC3OC3C2)c1=O ; O=C(CCCn1c(=O)n(CCO)c(=O)n(CCCC(=O)OCC2CCC3OC3C2)c1=O)OCC1CCC2OC2C1 ; O=C(O)Cn1c(=O)n(CCCC(=O)OCC2CCC3OC3C2)c(=O)n(CCCC(=O)OCC2CCC3OC3C2)c1=O]

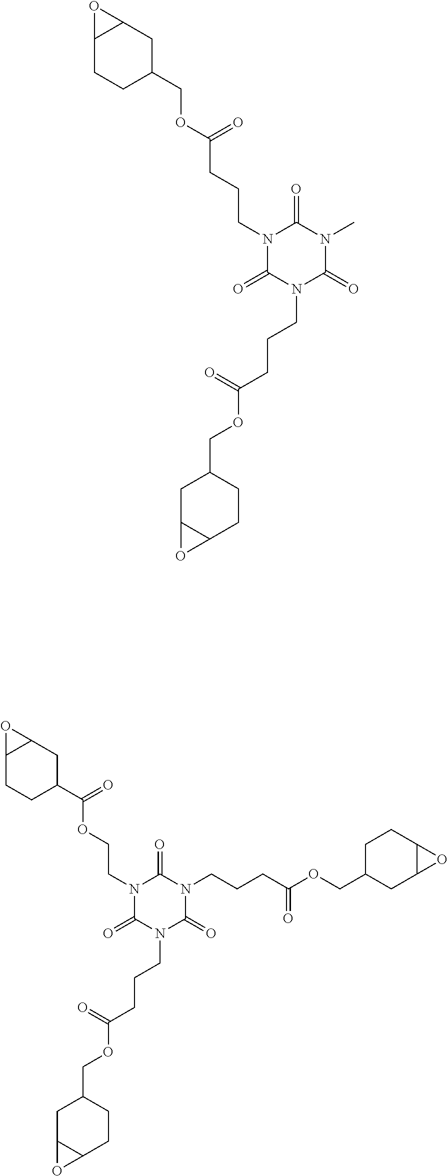 [SMILES: Cn1c(=O)n(CCCC(=O)OCC2CCC3OC3C2)c(=O)n(CCCC(=O)OCC2CCC3OC3C2)c1=O ; O=C(CCCn1c(=O)n(CCCC(=O)OCC2CCC3OC3C2)c(=O)n(CCOC(=O)C2CCC3OC3C2)c1=O)OCC1CCC2OC2C1]

In addition, a siloxane compound having two or more groups selected from a glycidyl group and an alicyclic epoxy group in the molecule (hereinafter simply referred to as “siloxane compound”) can be suitably used as the epoxy compound.

The siloxane compound is a compound including a siloxane skeleton composed of a siloxane bond (Si—O—Si) and two or more groups selected from the glycidyl group and the alicyclic epoxy group in the molecule.

Examples of the siloxane skeleton in the siloxane compound include a cyclic siloxane skeleton, and a cage or ladder type polysilsesquioxane skeleton.

The siloxane compound is preferably a compound including a cyclic siloxane skeleton represented by the following formula (a1-III) (hereinafter sometimes referred to as “cyclic siloxane”).

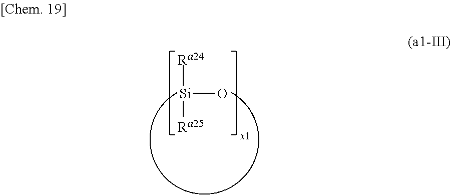

In the formula (a1-III), R^{a24} and R^{a25} represent a monovalent group having an epoxy group, or an alkyl group. Provided that, at least two of x1 R^{a24}s and x1 R^{a25}s in the compound represented by the formula (a1-III) is a monovalent group having a glycidyl group. In addition, x1 in the formula (a1-III) represents an integer of 3 or more. R^{a24} and R^{a25} in the compound represented by the formula (a1-III) may be the same or different. Plural R^{a24}s may be the same or different. Plural R^{a25}s may also be the same or different. Examples of the alkyl group include linear or branched alkyl groups having 1 or more and 18 or less carbon atoms (preferably 1 or more and 6 or less carbon atoms, and particularly preferably 1 or more and 3 or less carbon atoms) such as a methyl group, an ethyl group, a propyl group, and an isopropyl group.

x1 in the formula (a1-III) represents an integer of 3 or more, and preferably an integer of 3 or more and 6 or less in view of excellent crosslinking reactivity in the case of forming a functional material by using the composition. The number of epoxy groups possessed by the siloxane compound in the molecule is 2 or more, preferably 2 or more and 6 or less in view of excellent crosslinking reactivity in the case of forming a functional material by using the composition, and particularly preferably 2 or more and 4 or less.

An alicyclic epoxy group, and a glycidyl ether group represents by -D^{A}-O—R^{a26} [in which, D^{A} represents an alkylene group, and R^{a26} represents a glycidyl group] are preferable, an alicyclic epoxy group is more preferable, and an alicyclic epoxy group represented by the following formula (a1-IIIa) or the following formula (a1-IIIb) is further preferable as the monovalent group including the epoxy group. Examples of the D^{A} (the alkylene group) include a linear or branched alkylene group having 1 or more and 18 or less carbon atoms such as a methylene group, methylmethylene group, dimethylmethylene group, dimethylene group, and trimethylene group.

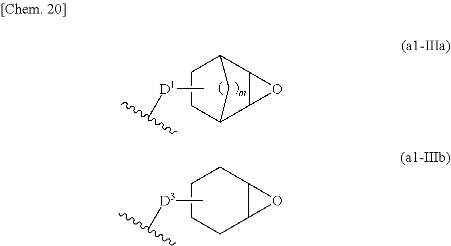

In the formula (a1-IIIa) and the formula (a1-IIIb), D¹ and D² each independently represent an alkylene group, and ms represents an integer of 0 or more and 2 or less.

The composition may include, in addition to the siloxane compound represented by the formula (a1-III), compounds including a siloxane skeleton, such as an alicyclic epoxy group-containing cyclic siloxane, an alicyclic epoxy group-containing silicone resin mentioned in Japanese Unexamined Patent Application, Publication No. 2008-248169, and an organopolysilsesquioxane resin having at least two epoxy functional groups in a molecule mentioned in Japanese Unexamined Patent Application, Publication No. 2008-19422 as the epoxy compound.

More specific examples of the siloxane compound include cyclic siloxane having two or more glycidyl groups in the molecule represented by the following formula. It is possible to use, as the siloxane compound, commercially available products, for example, trade name “X-40-2670”, “X-40-2701”, “X-40-2728”, “X-40-2738”, and “X-40-2740” (which are manufactured by Shin-Etsu Chemical Co., Ltd.).

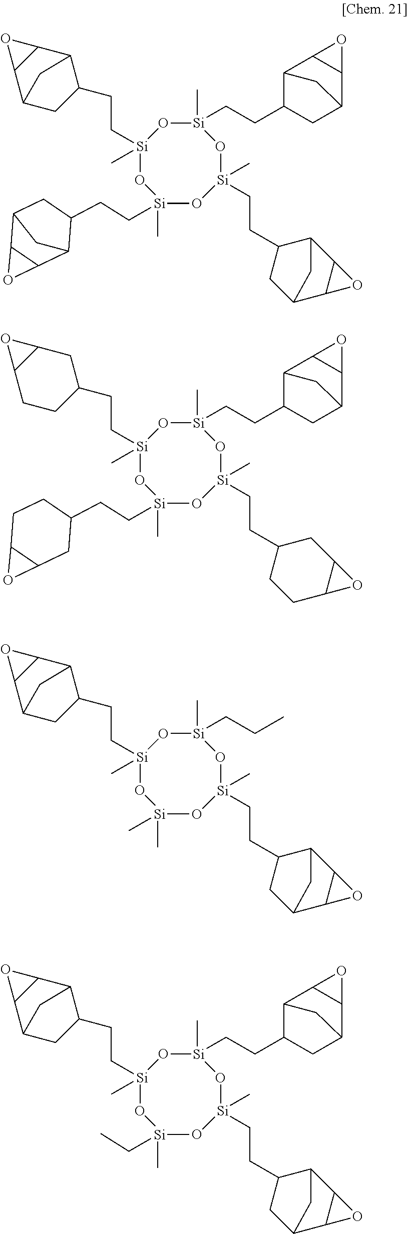 [SMILES: C[Si]1(CCC2CC3CC2C2OC32)O[Si](C)(CCC2CC3CC2C2OC32)O[Si](C)(CCC2CC3CC2C2OC32)O[Si](C)(CCC2CC3CC2C2OC32)O1 ; C[Si]1(CCC2CCC3OC3C2)O[Si](C)(CCC2CCC3OC3C2)O[Si](C)(CCC2CC3CC2C2OC32)O[Si](C)(CCC2CCC3OC3C2)O1 ; CCC[Si]1(C)O[Si](C)(CCC2CC3CC2C2OC32)O[Si](C)(C)O[Si](C)(CCC2CC3CC2C2OC32)O1 ; CC[Si]1(C)O[Si](C)(CCC2CC3CC2C2OC32)O[Si](C)(CCC2CC3CC2C2OC32)O[Si](C)(CCC2CC3CC2C2OC32)O1]

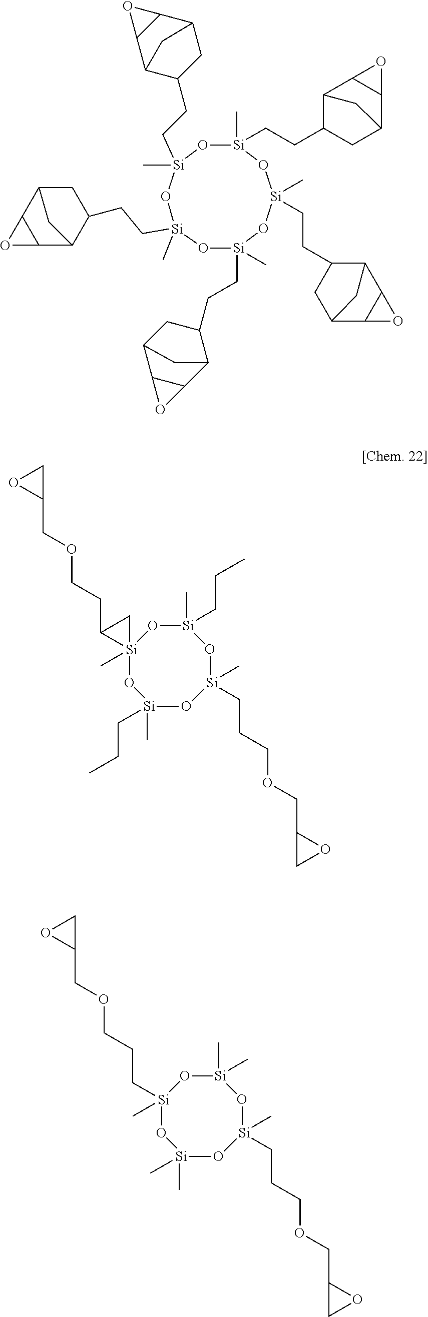

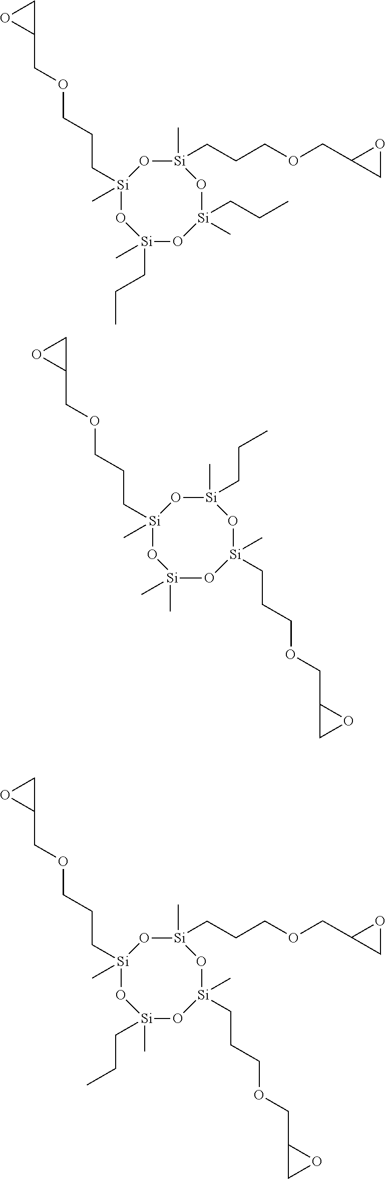 [SMILES: CCC[Si]1(C)O[Si](C)(CCC)O[Si](C)(CCCOCC2CO2)O[Si](C)(CCCOCC2CO2)O1 ; CCC[Si]1(C)O[Si](C)(CCCOCC2CO2)O[Si](C)(C)O[Si](C)(CCCOCC2CO2)O1 ; CCC[Si]1(C)O[Si](C)(CCCOCC2CO2)O[Si](C)(CCCOCC2CO2)O[Si](C)(CCCOCC2CO2)O1]

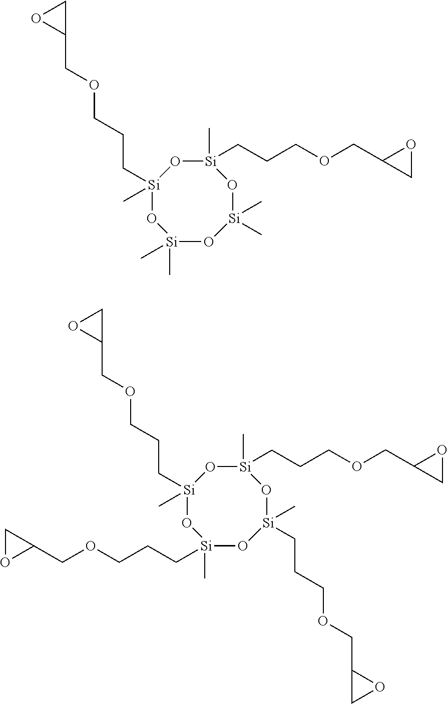 [SMILES: C[Si]1(C)O[Si](C)(C)O[Si](C)(CCCOCC2CO2)O[Si](C)(CCCOCC2CO2)O1 ; C[Si]1(CCCOCC2CO2)O[Si](C)(CCCOCC2CO2)O[Si](C)(CCCOCC2CO2)O[Si](C)(CCCOCC2CO2)O1]

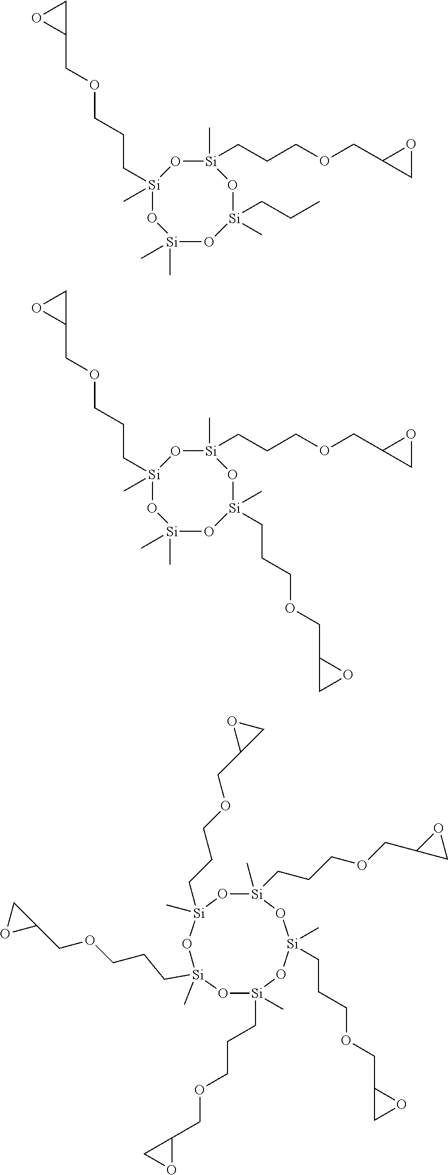 [SMILES: CCC[Si]1(C)O[Si](C)(C)O[Si](C)(CCCOCC2CO2)O[Si](C)(CCCOCC2CO2)O1 ; C[Si]1(C)O[Si](C)(CCCOCC2CO2)O[Si](C)(CCCOCC2CO2)O[Si](C)(CCCOCC2CO2)O1 ; C[Si]1(CCCOCC2CO2)O[Si](C)(CCCOCC2CO2)O[Si](C)(CCCOCC2CO2)O[Si](C)(CCCOCC2CO2)O[Si](C)(CCCOCC2CO2)O1]

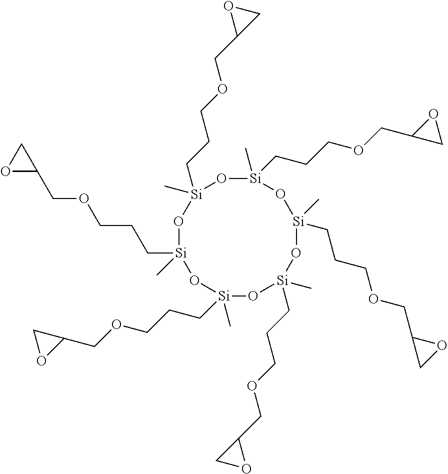 [SMILES: C[Si]1(CCCOCC2CO2)O[Si](C)(CCCOCC2CO2)O[Si](C)(CCCOCC2CO2)O[Si](C)(CCCOCC2CO2)O[Si](C)(CCCOCC2CO2)O[Si](C)(CCCOCC2CO2)O1]

In view of easy formation of a material having a high refractive index by using the composition, the composition preferably includes a compound represented by following formula (A-2e) as other photopolymerizable compound (A2).

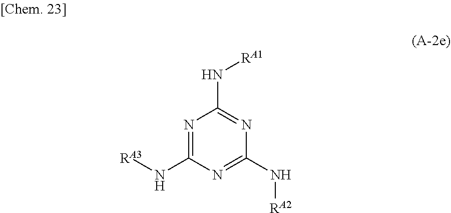

In the formula (A-2e), R^{A1}, R^{A2}, and R^{A3} are each independently an organic group. At least two of the organic group as R^{A1}, the organic group as R^{A2}, and the organic group as R^{A3} have the radically polymerizable group-containing group or the cationically polymerizable group-containing group.

Suitable examples of the compound represented by the formula (A-2e) include a compound represented by following formula (A-2e-a).

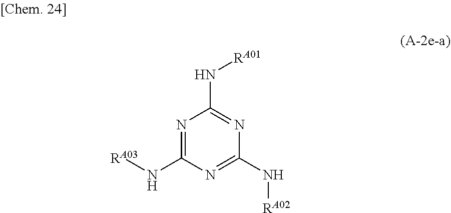

In the formula (A-2e-ad), R^{A01} is an optionally substituted quinolinyl group, an optionally substituted isoquinolinyl group, or an optionally substituted 2-substituted benzothiazolyl group. The 2-substituted benzothiazolyl group has a group represented by —S—R^{A0} at 2-position. R^{A0} is a hydrogen atom, a radically polymerizable group-containing group, or a cationically polymerizable group-containing group. Both of R^{A02} and R^{A03} are aromatic ring-containing group having the radically polymerizable group-containing group, or aromatic ring-containing group having the cationically polymerizable group-containing group. —NH— group bonding to triazine ring bonds to the aromatic rings in R^{A02} and R^{A03}.

All of the optionally substituted quinolinyl group, the optionally substituted isoquinolinyl group, and the optionally substituted 2-substituted benzothiazolyl group have large polarizability and small volume as a functional group. Therefore, it is thought that the optionally substituted quinolinyl group, the optionally substituted isoquinolinyl group, and the optionally substituted 2-substituted benzothiazolyl group give high refractive index to the material formed by using the composition.

The quinolinyl group as R^{A}01 may be any one of quinolin-2-yl group, quinolin-3-yl group, quinolin-4-yl group, quinolin-5-yl group, quinolin-6-yl group, quinolin-7-yl group, and quinolin-8-yl group. Among these groups, quinolin-3-yl group and quinolin-4-yl group are preferred in view of ease of obtaining raw material compound for the compound represented by the formula (A2e-a), ease of synthesizing the compound represented by the formula (A2e-a), and the like.

The isoquinolinyl group as R^{A01} may be any one of isoquinolin-1-yl group, isoquinolin-3-yl group, isoquinolin-4-yl group, isoquinolin-5-yl group, isoquinolin-6-yl group, isoquinolin-7-yl group, and isoquinolin-8-yl group. The isoquinolinyl group as R^{A01} may be any one of isoquinolin-1-yl group, isoquinolin-3-yl group, isoquinolin-4-yl group, isoquinolin-5-yl group, isoquinolin-6-yl group, isoquinolin-7-yl group, and isoquinolin-8-yl group.

The substituent which quinolinyl group and isoquinolinyl group as R^{A01} may have is not particularly limited as long as the desired effect is not impaired. Examples of substituent include a halogen atom, hydroxy group, mercapto group, cyano group, nitro group, and a monovalent organic group. Examples of the halogen atom as the substituent include fluorine atom, chlorine atom, bromine atom, and iodine atom. Examples of the monovalent organic group include an alkyl group, an alkoxy group, an alkoxyalkyl group, an aliphatic acryl group, an aliphatic acyloxy group, an alkoxycarbonyl group, an alkylthio group, an aliphatic acylthio group, and the like. In addition, the radically polymerizable group-containing group and the cationically polymerizable group-containing group are preferred as the monovalent organic group.

A number of carbon atoms in the monovalent organic group as the substituent is not particularly limited as long as the desired effect is not impaired. For example, the number of carbon atoms in the monovalent organic group as the substituent is preferably 1 or more and 20 or less, more preferably 1 or more and 12 or less, and further preferably 1 or more and 8 or less. In the alkoxyalkyl group, the aliphatic acyl group, the aliphatic acyloxy group, the alkoxycarbonyl group, the alkoxyalkylthio group, and the aliphatic acylthio group, lower limit of the number of carbon atoms is 2.

Suitable specific examples of the alkyl group as the substituent include methyl group, ethyl group, n-propyl group, isopropyl group, n-butyl group, isobutyl group, sec-butyl group, tert-butyl group, n-pentyl group, n-hexyl group, n-heptyl group, and n-octyl group.

Suitable specific examples of the alkoxy group as the substituent include methoxy group, ethoxy group, n-propyloxy group, isopropyl oxy group, n-butyloxy group, isobutyloxy group, sec-butyloxy group, tert-butyloxy group, n-pentyloxy group, n-hexyloxy group, n-heptyloxy group, and n-octyloxy group.

Suitable specific examples of the alkoxyalkyl group include methoxymethyl group, ethoxymethyl group, n-propyloxymethyl group, n-butyloxymethyl group, 2-methoxyethyl group, 2-ethoxyethyl group, 2-n-propyloxyethyl group, 2-n-butyloxyethyl group, 3-methoxy-n-propyl group, 3-ethoxy-n-propyl group, 3-n-propyloxy-n-propyl group, 3-n-butyloxy-n-propyl group, 4-methoxy-n-bnutyl group, 4-ethoxy-n-butyl group, 4-n-propyloxy-n-butyl group, and 4-n-butyloxy-n-butyl group.

Suitable specific examples of the aliphatic acyl group include acetyl group, propionyl group, butanoyl group, pentanoyl group, hexanoyl group, heptanoyl group, and octanoyl group.

Suitable specific examples of the aliphatic acyloxy group as the substituent include acetoxy group, propionyloxy group, butanoyloxy group, pentanoyloxy group, hexanoyloxy group, heptanoyloxy group, and octanoyloxy group.

Suitable specific examples of the alkoxycarbonyl group as the substituent include methoxycarbonyl group, ethoxycarbonyl group, n-propyloxycarbonyl group, isopropyloxycarbonyl group, n-butyloxycarbonyl group, isobutyloxycarbonyl group, sec-butyloxycarbonyl group, tert-butyloxycarbonyl group, n-pentyloxycarbonyl group, n-hexyloxycarbonyl group, n-heptyloxycarbonyl group, and n-octyloxycarbonyl group.

Suitable specific examples of the alkylthio group as the substituent include methylthio group, ethylthio group, n-propylthio group, isopropylthio group, n-butylthio group, isobutylthio group, sec-butylthio group, tert-butylthio group, n-pentylthio group, n-hexylthio group, n-heptylthio group, and n-octylthio group.

Suitable specific examples of the aliphatic acylthio group as the substituent include acetylthio group, propionylthio group, butanoylthio group, pentanoylthio group, hexanoylthio group, heptanoylthio group, and ocatnoylthio group.

When the quinolinyl group and the isoquinolinyl group have the substituent, a number of the substituent is not particularly limited as long as the desired effect is not impaired. When the quinolinyl group and the isoquinolinyl group have the substituent, the number of the substituent is preferably 1 or more and 4 or less, more preferably 1 or 2, and particularly preferably 1. When the quinolinyl group and the isoquinolinyl group have a plurality of the substituents, the plurality of the substituents may be different from each other.

The 2-substituted benzothiazoly group as R^{A0} has the group represented by —S—R^{A0} at 2-position. The 2-substituted benzothiazoly group as R^{A01} may have other substituent than the group represented by —S—R^{A0} at other position than 2-position. is a hydrogen atom, a radically polymerizable group-containing group, or a cationically polymerizable group-containing group. The radically polymerizable group-containing group or the cationically polymerizable group-containing group will be described later.

Suitable examples of the 2-substituted benzothiazolyl group include the following group.

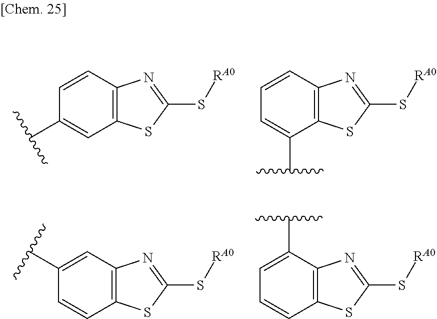

The substituent which the 2-substituted benzothiazolyl group as R^{A01} may have is the same as the substituent which the quinolinyl group and the isoquinolinyl group may have. When the 2-substituted benzothiazolyl group has the substituent, a number of the substituent is not particularly limited as long as the desired effect is not impaired. When the 2-substituted benzothiazolyl group has the substituent, the number of the substituent is preferably 1 or 2, and more preferably 1. When the 2-substituted benzothiazolyl group has a plurality of the substituents, the plurality of the substituents may be different from each other.

Both of R^{A02} and R^{A03} are aromatic ring-containing group having the radically polymerizable group-containing group, or aromatic ring-containing group having the cationically polymerizable group-containing group. It should be noted that —NH— group bonding to triazine ring bonds to the aromatic rings in R^{A02} and R^{A03}. In the aromatic ring-containing group as R^{A02} and R^{A03}, a position to which the radically polymerizable group-containing group or the cationically polymerizable group-containing group bonds is not particularly limited.

A number of the radically polymerizable group-containing group or the cationically polymerizable group-containing group in the aromatic ring-containing group as R^{A02}, and a number of the radically polymerizable group-containing group or the cationically polymerizable group-containing group in the aromatic ring-containing group as R^{A03} are not particularly limited. The number of the radically polymerizable group-containing group or the cationically polymerizable group-containing group in the aromatic ring-containing group as R^{A02}, and the number the radically polymerizable group-containing group or the cationically polymerizable group-containing group in the aromatic ring-containing group as R^{A03} are preferably an integer of 1 or more and 3 or less, more preferably 1 or 2, and particularly preferably 1.

The aromatic ring-containing group as R^{A02} and R^{A03} may include only one monocyclic aromatic ring or only one condensed aromatic ring, or two or more of monocyclic aromatic ring(s) and/or condensed aromatic ring. When the aromatic ring-containing group as R^{A02} and R^{A03} includes two or more of monocyclic aromatic rings and/or condensed aromatic rings, a type of bridging group which bridges monocyclic aromatic rings, condensed aromatic rings, or monocyclic aromatic ring and condensed aromatic ring. The bridging group may be a divalent bridging group or a trivalent bridging group, and is preferably a divalent bridging group.

Examples of the divalent bridging group include a divalent aliphatic hydrocarbon group, a divalent halogenated aliphatic hydrocarbon group, —CONH—, —NH—, —N═N—, —CH═N—, —COO—, —O—, —CO—, —SO—, —SO₂—, —S—, and —S—S—, and a combination of two or more of these.

In addition, a group represented by —CR^{a001}R^{a002}— is preferred as the divalent bridging group. R^{a001} and R^{a002} are each independently a hydrogen atom, an alkyl group having 1 or more and 4 or less carbon atoms, or a halogenated alkyl group having 1 or more and 4 or less carbon atoms. R^{a001} and R^{a002} may be combined with each other to form a ring. Specific examples of the group represented by —CR^{a001}R^{a002}— include methylene group, ethane-1,2-diyl group, propane-2,2-diyl group, butane-2,2-diyl group, 1,1,1,3,3,3-hexafluoropropane-2,2-diyl group, cyclopentylidene group, cyclohexylidene group, and cycloheptylidene group.

The aromatic ring containing-group as R^{A02} and R^{A03} has the radically polymerizable group-containing group or the cationically polymerizable group-containing group. The radically polymerizable group-containing group and the cationically polymerizable group-containing group are as described above.

Suitable examples of the radically polymerizable group-containing group include a group represented by the following formula (A-I) or the following formula (A-II) and not corresponding to vinyloxy group.

-(A⁰¹)ₙₐ-R⁰¹ (A-I)

-(A⁰¹)ₙₐ-R⁰²-A⁰²-R⁰¹ (A-II)

In the formula (A-I) and the formula (A-II), R⁰¹ is an alkenyl group having 2 or more and 10 or less carbon atoms. R⁰² is an alkylene group having 1 or more and 10 or less carbon atoms. A⁰¹ is —O—, —S—, —CO—, —CO—O—, —CO—S—, —O—CO—, —S—CO—, —CO—NH—, —NH—CO—, or —NH. A⁰² is —O—, —S—, —CO—, —CO—O—, —CO—S—, —O—CO—, —S—CO—, —CO—NH—, —NH—CO—, or —NH. na is 0 or 1.

Suitable specific examples of the radically polymerizable group-containing group include groups represented by
O—R⁰³,
S—R⁰³,
O—CH₂CH₂—O—R⁰³,
—O—CH₂CH₂CH₂—O—R⁰³,
—O—CH₂CH₂CH₂CH₂—O—R⁰³,
—COO—CH₂CH₂—O—R⁰³,
—CO—O—CH₂CH₂CH₂—O—R⁰³,
—COO—CH₂CH₂CH₂CH₂—O—R⁰³,
—O—CH₂CH₂—NH—R⁰³,
—O—CH₂CH₂CH₂—NH—R⁰³,
—O—CH₂CH₂CH₂CH₂—NH—R⁰³,
—CO—O—CH₂CH₂—NH—R⁰³,
—CO—O—CH₂CH₂CH₂—NH—R⁰³,
—CO—O—CH₂CH₂CH₂CH₂—R⁰³,
—NH—R⁰³,
—NH—CH₂CH₂—O—R⁰³,
—NH—CH₂CH₂CH₂—O—R⁰³,
—NH—CH₂CH₂CH₂CH₂—O—R⁰³,
—CO—NH—CH₂CH₂—O—R⁰³,
—CO—NH—CH₂CH₂CH₂—O—R⁰³,
—CO—NH—CH₂CH₂CH₂CH₂—O—R⁰³,
—NH—CH₂CH₂—NH—R⁰³,
—NH—CH₂CH₂CH₂—NH—R⁰³,
—NH—CH₂CH₂CH₂CH₂—NH—R⁰³,
—CO—NH—CH₂CH₂—NH—R⁰³,
—CO—NH—CH₂CH₂CH₂—NH—R⁰³, and
—CO—NH—CH₂CH₂CH₂CH₂—NH—R⁰³. R⁰³ in these groups is ally group or (meth)acryloyl group.

Suitable examples of the cationically polymerizable group-containing group include vinyl oxy group and groups represented by following formulas (A3) to (A8).

-(A⁰¹)ₙₐ-R⁰⁴ (A3)

-(A⁰¹)ₙₐ-R⁰²—R⁰⁵ (A4)

-(A⁰¹)ₙₐ-R⁰²—(CO)_{nb}-A⁰³-R⁴ (A5)

-(A⁰¹)ₙₐ-R⁰²—(CO)_{nb}-A⁰³-R⁰⁷—R⁰⁵ (A6)

-(A⁰¹)ₙₐ-R⁰²—O—R⁰⁶ (A7)

-(A⁰¹)ₙₐ-R⁰²—(CO)_{nb}-A³-R—O—R⁰⁶ (A8)

In the formulas (A³) to (A⁸), R⁰² is an alkylene group having 1 or more and 10 or less carbon atoms. R⁰⁴ is an epoxyalkyl group having 2 or more and 20 or less carbon atoms or an alicyclic epoxy group having 3 or more and 20 or less carbon atoms. R⁰⁵ is an alicyclic epoxy group having 3 or more and 20 or less carbon atoms. R⁰⁶ is vinyl group. R⁰⁷ is an alkylene group having 1 or more and 10 or less carbon atoms. A⁰¹ is —O—, —S—, —CO—, —CO—O—, —CO—S—, —O—CO—, —S—CO—, —CO—NH—, —NH—CO—, or —NH. A⁰³ is —O— or —NH—. nb is 0 or 1.

Suitable examples of the cationically polymerizable group-containing group include groups represented by
—O—CH₂CH₂—R⁰⁸,
—O—CH₂CH₂CH₂—R⁰⁸,
—O—CH₂CH₂CH₂CH₂—R⁰⁸,
—CO—O—CH₂CH₂—R⁰⁸,
—CO—O—CH₂CH₂CH₂—R⁰⁸,
—CO—O—CH₂CH₂CH₂CH₂—R⁰⁸,
—NH—CH₂CH₂—R⁰⁸,
—NH—CH₂CH₂CH₂—R⁰⁸,
—NH—CH₂CH₂CH₂CH₂—R⁰⁸,
—CO—NH—CH₂CH₂—R⁰⁸,
—CO—NH—CH₂CH₂CH₂—R⁰⁸, and
—CO—NH—CH₂CH₂CH₂CH₂—R⁰⁸. R⁰⁸ in these groups is vinyloxy group, glycidyloxy group, glycidylthio group, epoxycyclopentyl group, epoxycyclohexyl group, or epoxycycloheptyl group.

When the aromatic ring-containing group as R^{A02} and R^{A03} has one radically polymerizable group-containing group or one cationically polymerizable group-containing group, suitable examples of the R^{A02} and R^{A03} include following groups. In following formulas, PG is the radically polymerizable group-containing group or the cationically polymerizable group-containing group.

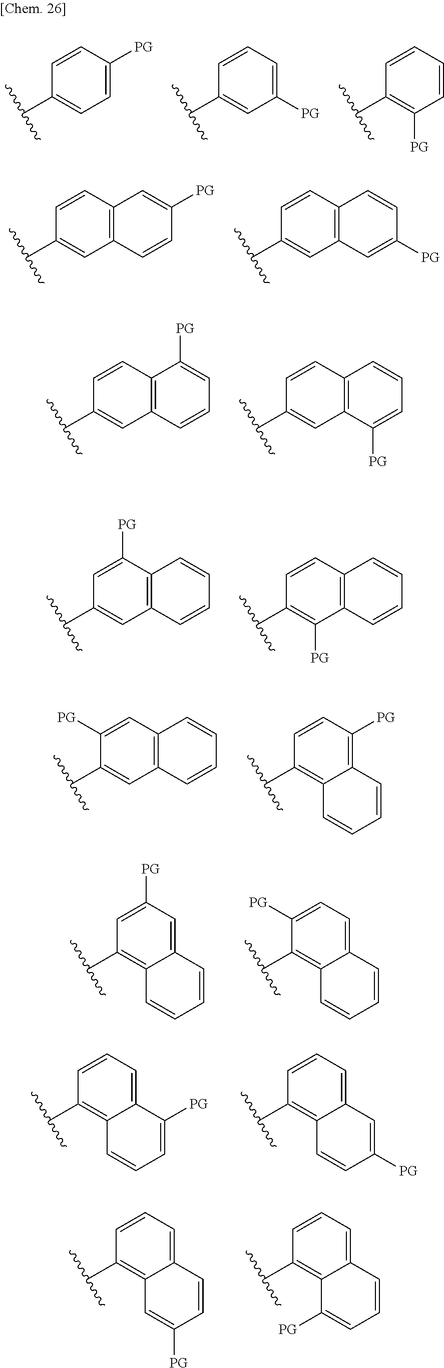

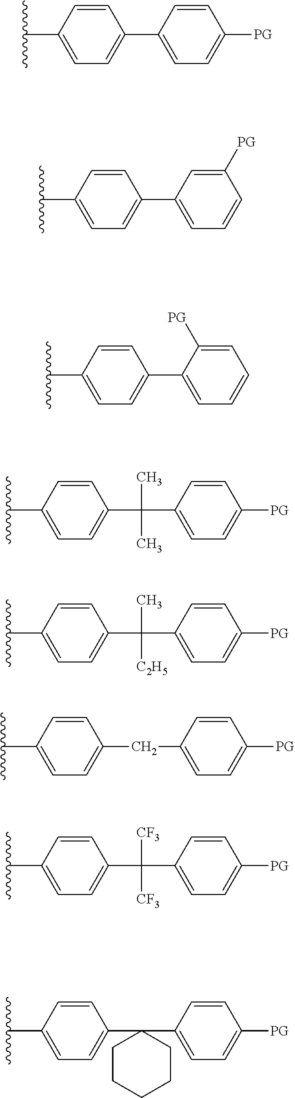

Suitable specific examples of the compound represented by the formula (A-2e) include the following compounds. In the following formulas, X^{A} is a group selected from the group consisting of (meth)acryloyloxy group, (meth)acryloylthio group, 3-(meth)acryloyloxy-2-hydroxy-n-propyloxycarbonyl group, and glycidyloxy group.

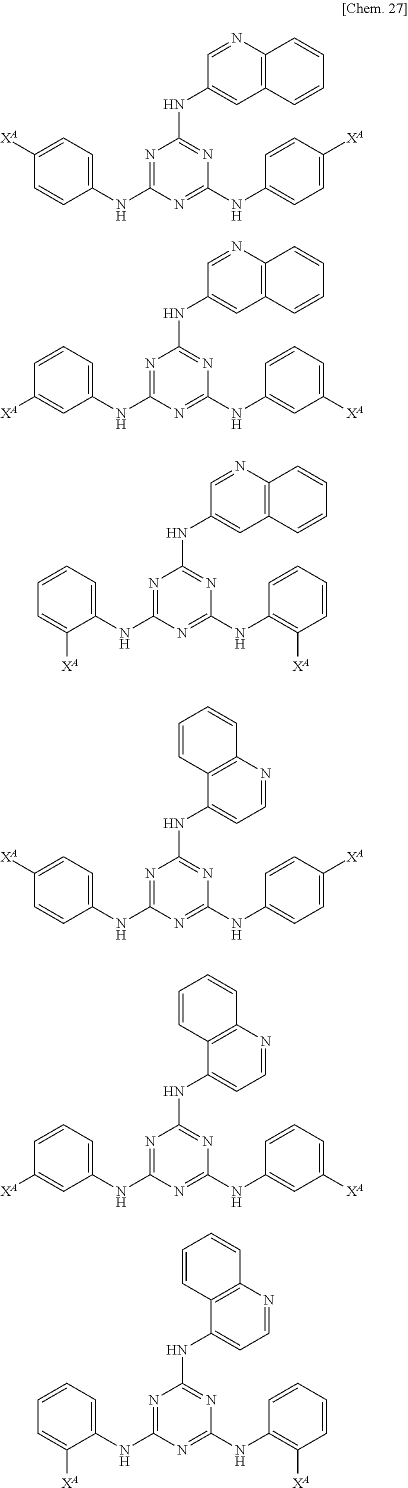

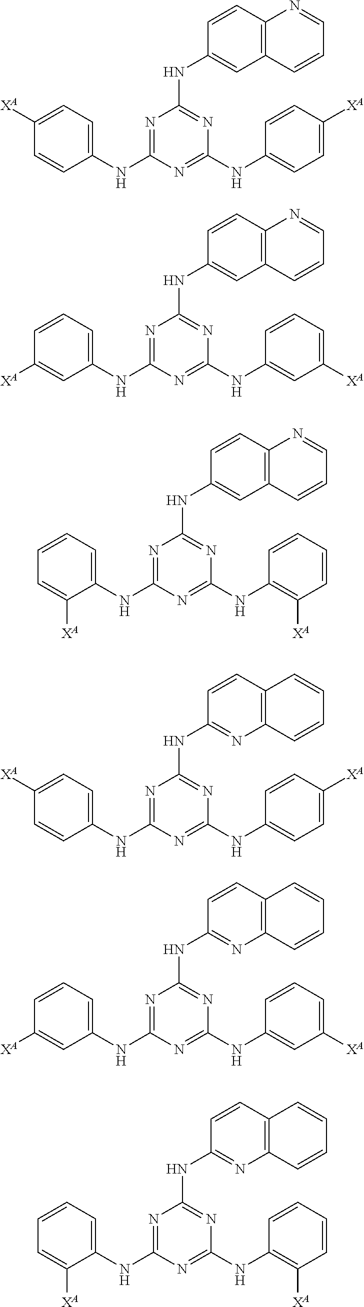

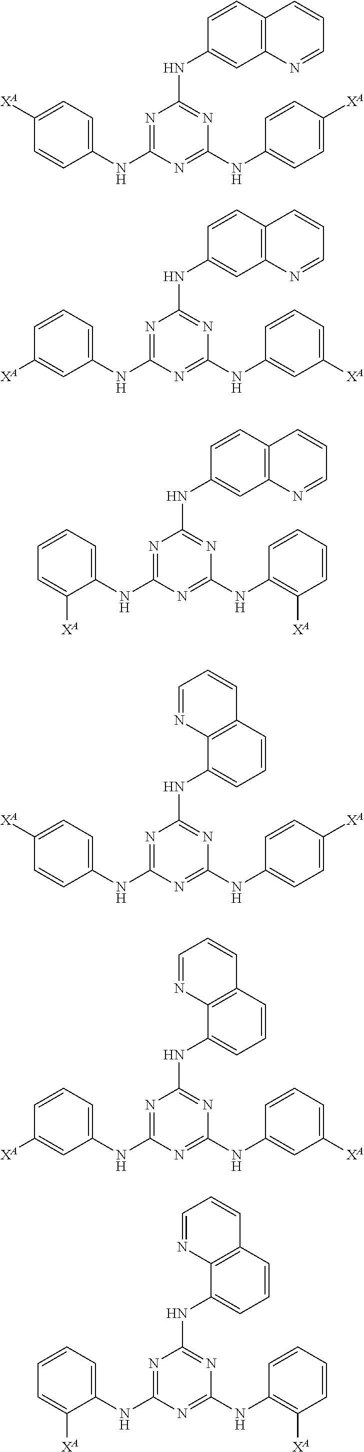

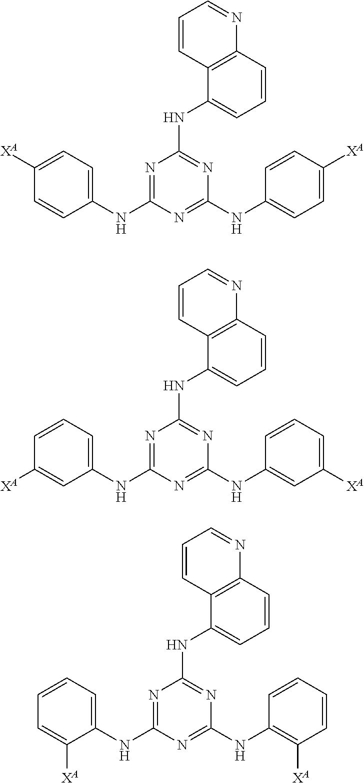

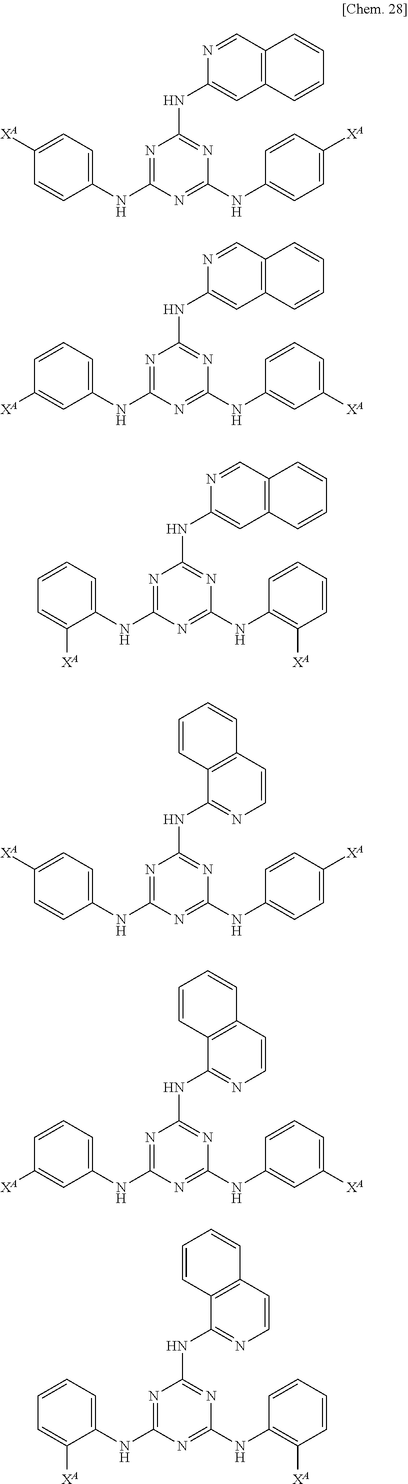

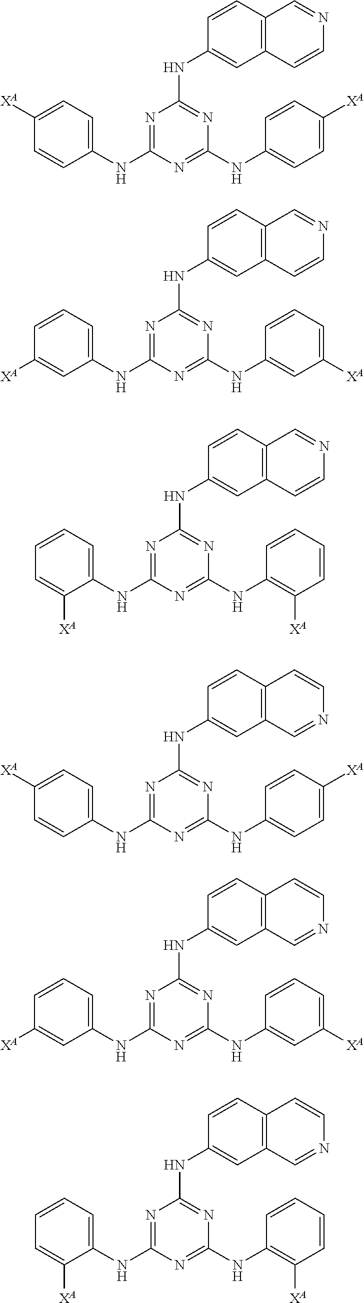

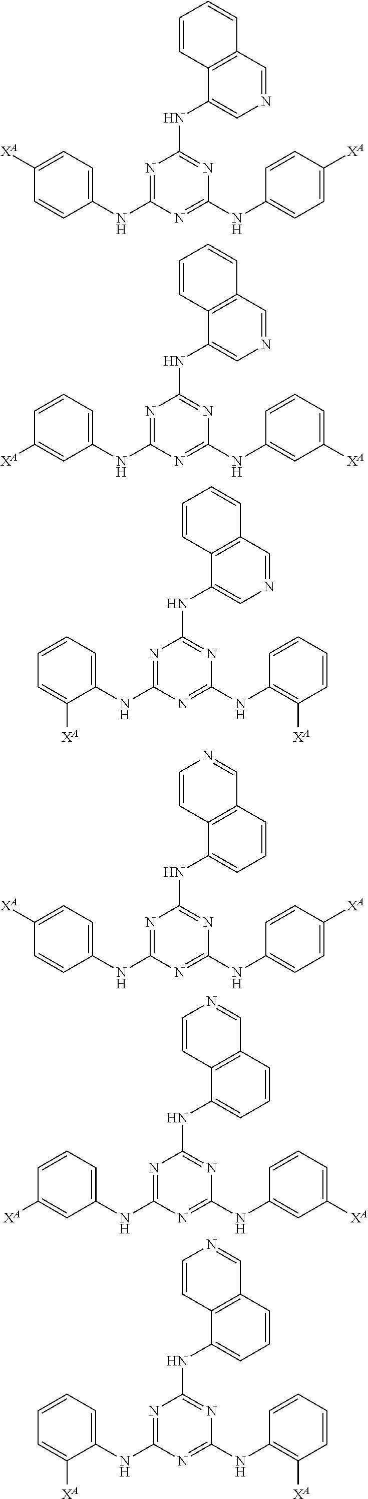

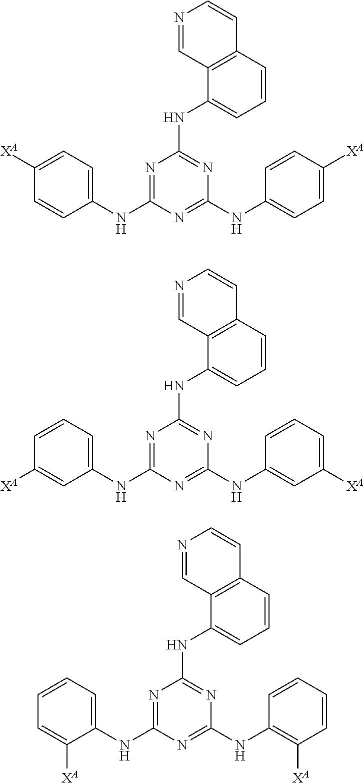

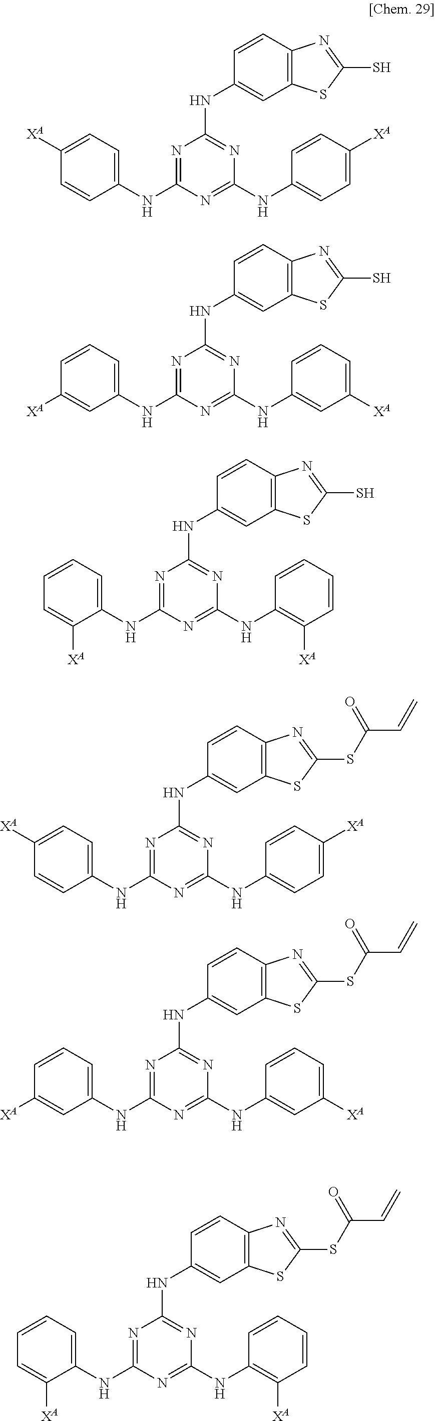

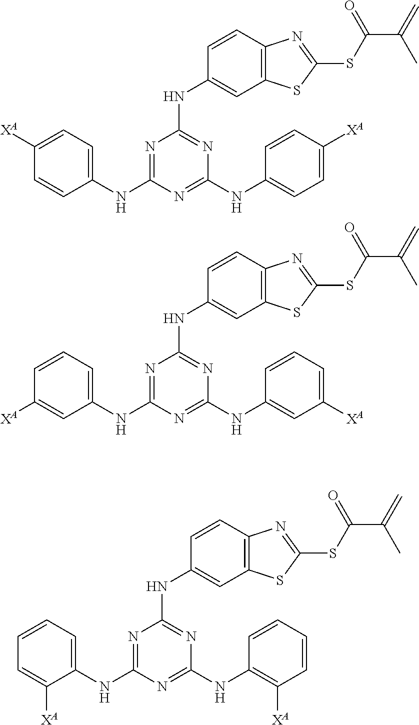

A production method of the compound represented by the formula (A-2e-a) is not particularly limited. Typically, the compound represented by the formula (A-2e-a) can be prepared by reacting cyanuric halide such as cyanuric chloride with aromatic amines represented by R^{A01}—NH₂, R^{A02}—NH₂, and R^{A03}—NH₂. These multiple amines may react with the cyanuric halide simultaneously or sequentially, and preferably sequentially react with the cyanuric halide.

In addition, R^{A02} and R^{A03} in the formula (A-2e-a) can be formed by reacting the cyanuric halide with aromatic amine having a functional group such as hydroxy group, mercapto group, carboxy group, and amino group, and thereafter reacting these functional groups with a compound which gives the radically polymerizable group-containing group or the cationically polymerizable group-containing group. Examples of the compound which gives the radically polymerizable group-containing group or the cationically polymerizable group-containing group include compounds having polymerizable group such as (meth)acrylic acid, (meth)acrylic acid halide, halogenated olefin, epichlorohydrin, and glycidyl (meth)acrylate. As a reaction of the functional group such as hydroxy group, mercapto group, carboxy group, and amino group with a compound having a polymerizable group, well-known reaction forming an ether bond, a carboxylic acid ester bond, a carboxylic amid bond, or a thioether bond can be used.

A reaction forming the radically polymerizable group-containing group or the cationically polymerizable group-containing group may be a multi-step reaction. For example, a radically polymerizable group represented by the following formula can be introduced on an aromatic ring by glycidylizing a phenolic hydroxy group by reaction with epichlorohydrin after reacting cyanuric halide with an aromatic amine having phenolic hydroxy group, subsequently, reacting the glycidyl group with acrylic acid.

—O—CH₂—CHOH—CH₂—O—CO—CH═CH₂

The above reaction is an example, and the radically polymerizable group-containing group or the cationically polymerizable group-containing group can be formed by carrying out various reactions in combination.

The compound represented by the formula (A-2e-a) is usually synthesized in an organic solvent. This organic solvent is not particularly limited as long as the solvent is an inactive solvent which does not react with cyanuric halide, aromatic amine, radically polymerizable group, cationically polymerizable group, and the like. As the solvent, solvents exemplified as specific examples of the solvent (S) described below can be used. In the production of the compound represented by the formula (A-2e-a), reaction temperature is not particularly limited when cyanuric halide is reacted with aromatic amines such as aromatic amines represented by R^{A01}—NH₂, R^{A02}—NH₂, and R^{A03}—NH₂. Typically, reaction temperature is preferably 0° C. or higher and 150° C. or lower.

Other suitable examples of the compound represented by the formula (A-2e) include a compound represented by the following formula (A-2e-b).

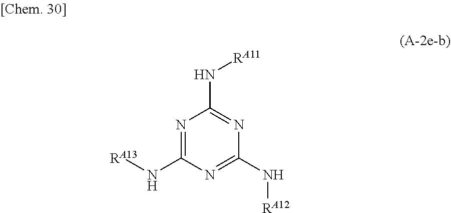

As described above, in the formula (A-2e-b), each of R^{A11}, R^{A12}, and R^{A13} is the aromatic ring-containing group. At least one of R^{A12} and R^{A13} is a group represented by following formula (A-2e-b1).

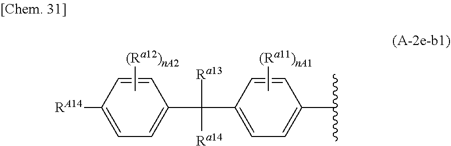

Each of the —NH— groups bonding to the triazine ring bonds to the aromatic ring in R^{A11}, R^{A12} and R^{A13}. In the formula (A-2e-b1), R^{a11} and R^{a12} are each independently an alkyl group having 1 or more and 4 or less carbon atoms, an alkoxy group having 1 or more and 4 or less carbon atoms, or a halogen atom.

nA1 and nA2 are each independently an integer of 0 or more and 4 or less. R^{a13} and R^{a14} are each independently an alkyl group having 1 or more and 4 or less carbon atoms, a halogenated alkyl group having 1 or more and 4 or less carbon atoms, or phenyl group. R^{a13} and R^{a14} may be combined with each other to form a ring. R^{A14} is the radically polymerizable group-containing group or the cationically polymerizable group-containing group. When both of R^{A12} and R^{A13} are the group represented by the formula (A-2e-b1), both of R^{A12} and R^{A13} have the radically polymerizable group-containing group or the cationically polymerizable group-containing group.

As described above, in the formula (A-2e-b), each of R^{A11}, R^{A12}, and R^{A13} is the aromatic ring-containing group. Each of —NH— groups bonding to the triazine ring in the formula (A-2e-b) bonds to the aromatic ring in R^{A11}, R^{A12}, and R^{A13}. When the aromatic ring-containing group is a group other than the group represented by the formula (A-2e-b1), the aromatic ring-containing group is not particularly limited as long as the above specific requirements are met.

The aromatic ring-containing group other than the group represented by the formula (A-2e-b1) may have only one monocyclic aromatic ring or one condensed aromatic ring, or may have two or more of monocyclic aromatic ring and/or condensed aromatic ring. When the aromatic ring-containing group as R^{A02} and R^{A03} includes two or more of monocyclic aromatic rings and/or condensed aromatic rings, a type of bridging group which bridges monocyclic aromatic rings, condensed aromatic rings, or monocyclic aromatic ring and condensed aromatic ring. The bridging group may be a divalent bridging group or a trivalent bridging group, and is preferably a divalent bridging group.

Examples of the divalent bridging group include a divalent aliphatic hydrocarbon group, a divalent halogenated aliphatic hydrocarbon group, —CONH—, —NH—, —N═N—, —CH═N—, —COO—, —O—, —CO—, —SO—, —SO₂—, —S—, and —S—S—, and a combination of two or more of these.

Suitable examples of the aromatic ring-containing group include an optionally substituted quinolinyl group, an optionally substituted isoquinolinyl group, and an optionally substituted 2-substituted benzothiazolyl group. These groups are the same as the optionally substituted quinolinyl group, the optionally substituted isoquinolinyl group, and the optionally substituted 2-substituted benzothiazolyl group described for R^{A01} in the formula (A-2e-a).

Other suitable examples of the aromatic ring-containing group include an optionally substituted phenyl group, an optionally substituted naphthyl group, an optionally substituted biphenylyl group, an optionally substituted phenylthiophenyl group, an optionally substituted phenoxyphenyl group, an optionally substituted phenylsulfonylphenyl group, an optionally substituted benzothiazolyl group, an optionally substituted benzoxazolyl group, and an optionally substituted terphenyl group. When these groups have substituent, the substituent is the same as the substituent which the quinolinyl group and the isoquinolinyl group may have. When these groups have a plurality of the substituents, the plurality of the substituents may be different from each other.

Suitable specific examples of the optionally substituted phenyl group include phenyl group, 4-cyanophenyl group, 3-cyanophenyl group, 2-cyanophenyl group, 2,3-dicyanophenyl group, 2,4-dicyanophenyl group, 2,5-dicyanophenyl group, 2,6-dicyanophenyl group, 3,4-dicyanophenyl group, 3,5-dicyanopohenyl group, 4-nitrophenyl group, 3-nitrophenyl group, 2-nitrophenyl group, 4-chlorophenyl group, 3-chlorophenyl group, 2-chlorophenyl group, 4-bromophenyl group, 3-bromophenyl group, 2-bromophenyl group, 4-iodophenyl group, 3-iodophenyl group, 2-iodophenyl group, 4-methoxyphenyl group, 3-methoxyphenyl group, 2-methoxyphenyl group, 4-methylphenyl group, 3-methylphenyl group, and 2-methylphenyl group.

Suitable specific examples of the optionally substituted naphthyl group include naphthalen-1-yl group, and naphthalen-2-yl group

Suitable examples of the optionally substituted biphenylyl group include 4-phenylphenyl group, 3-phenylphenyl group, 2-phenylphenyl group, 4-(4-nitrophenyl)phenyl group, 3-(4-nitrophenyl)phenyl group, 2-(4-nitrophenyl)phenyl group, 4-(4-cyanophenyl)phenyl group, 3-(4-cyanophenyl)phenyl group, and 2-(4-cyanophenyl)phenyl group.

Suitable specific examples of the optionally substituted phenylthiophenyl group include 4-phenylthiophenyl group, 3-phenylthiophenyl group, and 2-phenylthiophenyl group.

Suitable specific examples of the optionally substituted phenoxyphenyl group include 4-phenoxyphenyl group, 3-phenoxyphenyl group, and 2-phenoxyphenyl group.

Suitable specific examples of the optionally substituted phenylsulfonylphenyl group include 4-phenylsulfonylphenyl group, 3-phenylsulfonylphenyl group, and 2-phenylsulfonylphenyl group.

Suitable specific examples of the optionally substituted benzothiazolyl group include benzothiazol-2-yl group, benzothiazol-4-yl group, benzothiazol-5-yl group, benzothiazol-6-yl group, and benzothiazol-7-yl group.

Suitable specific examples of the optionally substituted benzoxazolyl group include benzoxazol-2-yl group, benzoxazol-4-yl group, benzoxazol-5-yl group, benzoxazol-6-yl group, and benzoxazol-7-yl group.

Suitable examples of the optionally substituted terphenyl group include 4-(4-phenylphenyl)phenyl group, 3-(4-phenylphenyl)phenyl group, 2-(4-phenylphenyl)phenyl group, 4-(3-phenylphenyl)phenyl group, 3-(3-phenylphenyl)phenyl group, 2-(3-phenylphenyl)phenyl group, 4-(2-phenylphenyl)phenyl group, 3-(2-phenylphenyl)phenyl group, and 2-(2-phenylphenyl)phenyl group.

As described above, the aromatic ring-containing group other than the group represented by the formula (A-2e-b1) may have the radically polymerizable group-containing group or the cationically polymerizable group-containing group as substituent. In the aromatic ring-containing group, a position to which the radically polymerizable group-containing group or the cationically polymerizable group-containing group bonds is not particularly limited.

A number of the radically polymerizable group-containing group or the cationically polymerizable group-containing group is not particularly limited. The number of the radically polymerizable group-containing group or the cationically polymerizable group-containing group in the aromatic group-containing group is preferably an integer of 1 or more and 3 or less, more preferably 1 or 2, and particularly preferably 1.

When the aromatic ring-containing group has the one radically polymerizable group-containing group or the one cationically polymerizable group-containing group, suitable examples of such group include following groups. In following formulas, PG is the radically polymerizable group-containing group or the cationically polymerizable group-containing group.

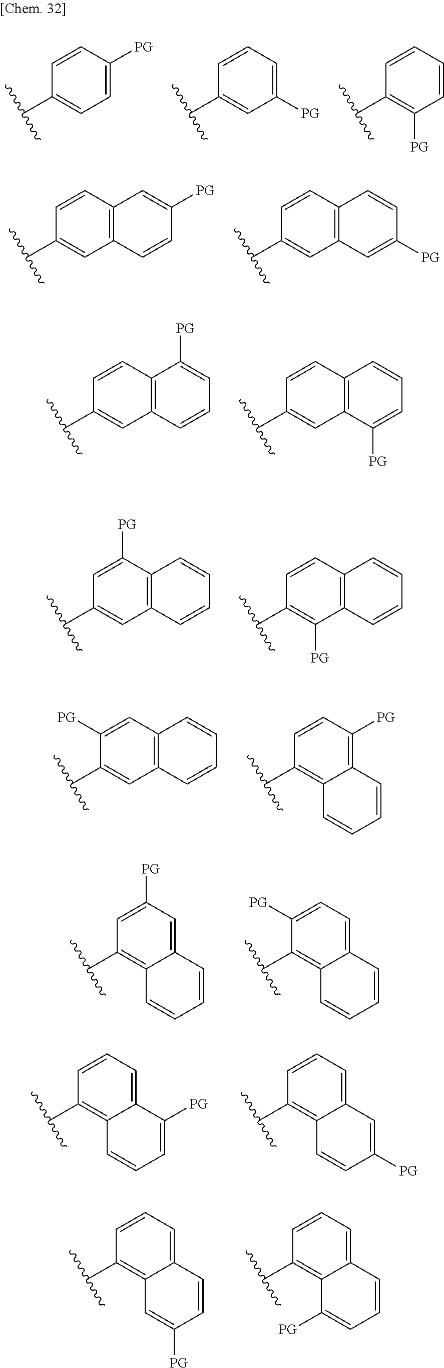

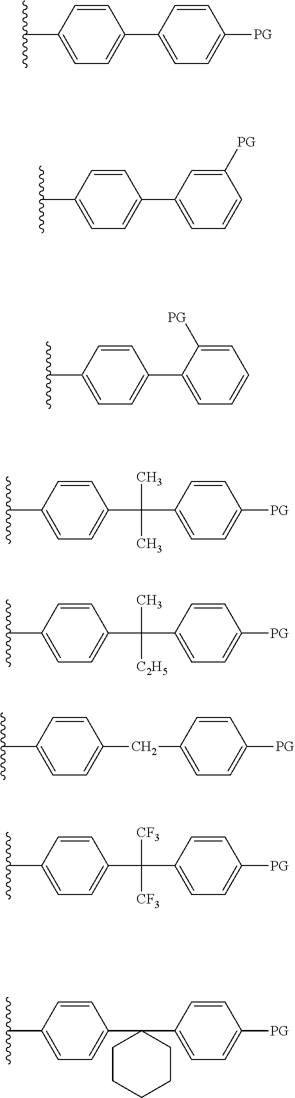

In the formula (A-2e-b), at least one of R^{A12} and R^{A13} is a group represented by following formula (A-2e-b1).

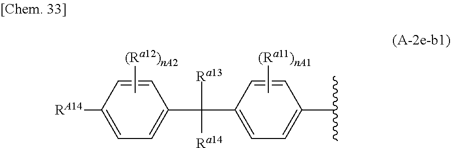

In the formula (A-2e-b1), R^{a11} and R^{a1}2 are each independently an alkyl group having 1 or more and 4 or less carbon atoms, an alkoxy group having 1 or more and 4 or less carbon atoms, or a halogen atom. nA1 and nA2 are each independently an integer of 0 or more and 4 or less. R^{a1}3 and R^{a14} are each independently an alkyl group having 1 or more and 4 or less carbon atoms, a halogenated alkyl group having 1 or more and 4 or less carbon atoms, or phenyl group. R^{a13} and R^{a14} may be combined with each other to form a ring. R^{A14} is the radically polymerizable group-containing group or the cationically polymerizable group-containing group. When both of R^{A12} and R^{A13} are the group represented by the formula (A-2e-b1), both of R^{A12} and R^{A13} have the radically polymerizable group-containing group or the cationically polymerizable group-containing group.

Examples of the alkyl group having 1 or more and 4 or less carbon atoms as R^{a11} and R^{a12} include methyl group, ethyl group, n-propyl group, isopropyl group, n-butyl group, isobutyl group, sec-butyl group, and tert-butyl group. Examples of the alkoxy group having 1 or more and 4 or less carbon atoms as R^{a11} and R^{a12} include methoxy group, ethoxy group, n-propyloxy group, isopropyloxy group, n-butyloxy group, isobutyloxy group, sec-butyloxy group, and tert-butyloxy group. Examples of the halogen atom as R^{a11} and R^{a12} include fluorine atom, chlorine atom, bromine atom, and iodine atom.

Specific examples of the alkyl group having 1 or more and 4 or less carbon atoms as R^{a13} and R^{a14} are the same as the specific examples of the alkyl group having 1 or more and 4 or less carbon atoms as R^{a11} and R^{a12}. Specific examples of the halogenated alkyl group having 1 or more and 4 or less carbon atoms as R^{a13} and R^{a14} include chloromethyl group, dichloromethyl group, trichloromethyl group, bromomethyl group, dibromomethyl group, tribromomethyl group, fluoromethyl group, difluoromethyl group, trifulurormethyl group, 3,3,3-trifluoroethyl group, pentafluoroethyl group, heptafluoropropyl group, and the like.

Suitable examples of the group represented by the formula (A-2e-b1) include the compounds represented by following formulas.

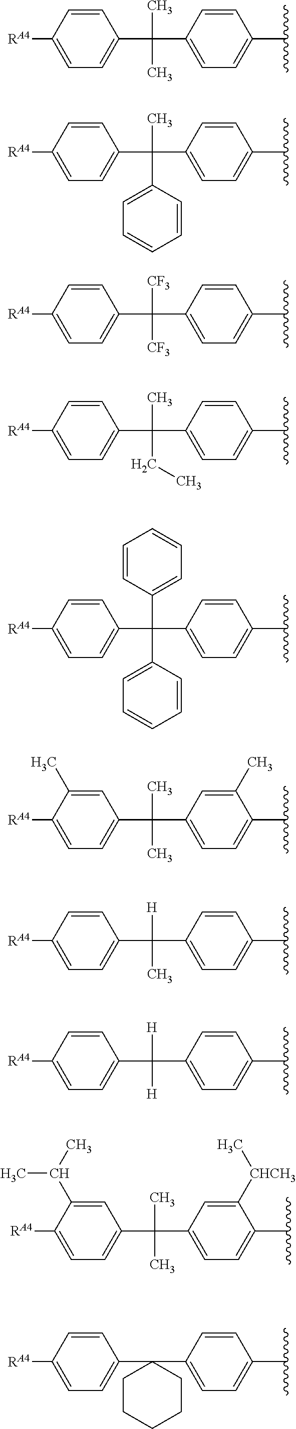

The group represented by the formula (A-2e-b1) has the radically polymerizable group-containing group or the cationically polymerizable group-containing group as R^{A14}. The radically polymerizable group-containing group and the cationically polymerizable group-containing group are as described above. Suitable specific examples of the radically polymerizable group-containing group, and suitable specific examples of the cationically polymerizable group-containing group are the same as the suitable specific examples of the radically polymerizable group-containing group, and the suitable specific examples of the cationically polymerizable group-containing group described for the compound represented by the formula (A-2e-a).

Suitable specific examples of the compound represented by the formula (A-2e-b) include the following compounds. In the following formulas, X^{A} is a group selected from the group consisting of (meth)acryloyloxy group, (meth)acryloylthio group, 3-(meth)acryloyloxy-2-hydroxy-n-propyloxycarbonyl group, and glycidyloxy group. Y^{A} is a group selected from the group consisting of quinolin-3-yl group, phenyl group, 4-cyanophenyl group, 3-cyanophenyl group, 2-cyanophenyl group, 3,4-dicyanophenyl group, 4-nitrophenyl group, 4-methoxyphenyl group, 4-phenylthiophenyl group, 4-phenylsulfonylphenyl, 4-iodophenyl group, benzothiazol-2-yl group, 2-mercaptobenzothiazol-5-yl group, 4-phenylphenyl group, 4-(4-nitrophenyl)phenyl group, 4-(4-cyanophenyl)phenyl group, naphthalen-1-yl group, and 4-(4-phenylphenyl)phenyl group.

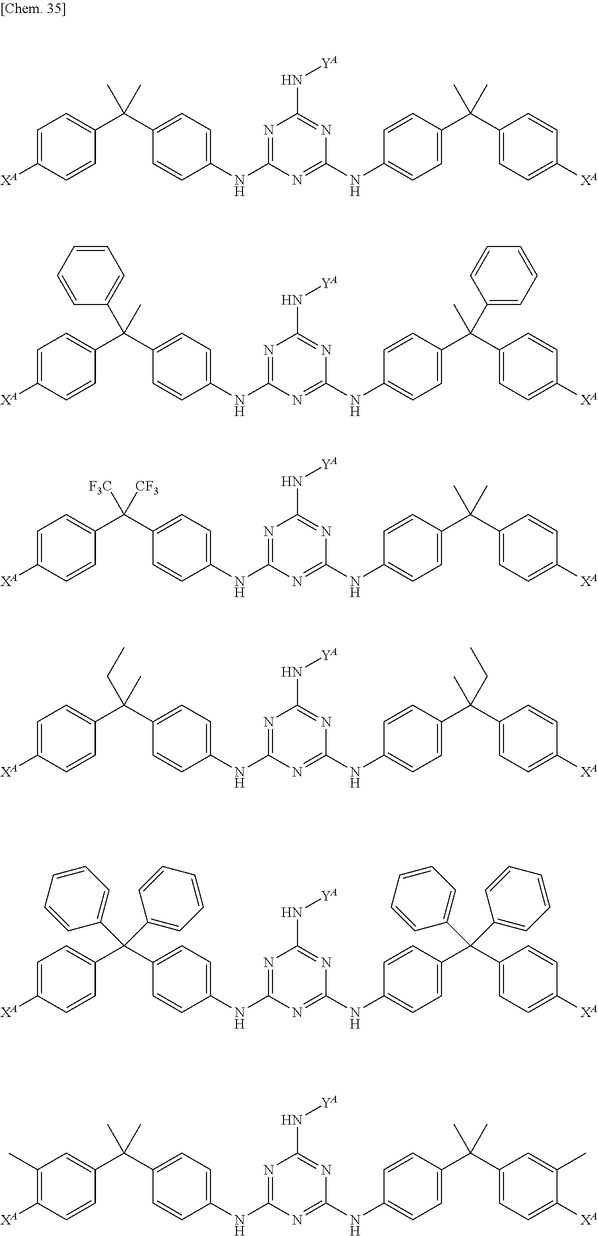

Production method of the compound represented by the formula (A-2e-b) is not particularly limited. Typically, the compound represented by the formula (A-2e-b) can be prepared by reacting cyanuric halide such as cyanuric chloride with aromatic amines represented by R^{A11}—NH₂, R^{A12}—NH₂, and R^{A13}—NH₂. These multiple amines may react with the cyanuric halide simultaneously or sequentially, and preferably sequentially react with the cyanuric halide.

In addition, when the aromatic ring-containing group bonding to the triazine ring via —NH— has the radically polymerizable group-containing group or the cationically polymerizable group-containing group, an aromatic amine having a functional group such as hydroxy group, mercapto group, carboxy group and amino group is reacted with the cyanuric halide. Thereafter, by reacting these functional groups with a compound which gives the radically polymerizable group-containing group or the cationically polymerizable group-containing group, the radically polymerizable group-containing group or the cationically polymerizable group-containing group can be formed. Examples of the compound which gives the radically polymerizable group-containing group or the cationically polymerizable group-containing group include compounds having polymerizable group such as (meth)acrylic acid, (meth)acrylic acid halide, halogenated olefin, epichlorohydrin, and glycidyl (meth)acrylate. As a reaction of the functional group such as hydroxy group, mercapto group, carboxy group, and amino group with a compound having a polymerizable group, well-known reaction forming an ether bond, a carboxylic acid ester bond, a carboxylic amid bond, or a thioether bond can be used.

A reaction forming the radically polymerizable group-containing group or the cationically polymerizable group-containing group may be a multi-step reaction. For example, a radically polymerizable group represented by the following formula can be introduced on an aromatic ring by glycidylizing a phenolic hydroxy group by reaction with epichlorohydrin after reacting cyanuric halide with an aromatic amine having phenolic hydroxy group, subsequently, reacting the glycidyl group with acrylic acid.

—O—CH₂—CHOH—CH₂—O—CO—CH═CH₂

The above reaction is an example, and the radically polymerizable group-containing group or the cationically polymerizable group-containing group can be formed by carrying out various reactions in combination.

The compound represented by the formula (A-2e-b) is usually synthesized in an organic solvent. This organic solvent is not particularly limited as long as the solvent is an inactive solvent which does not react with cyanuric halide, aromatic amine, radically polymerizable group, cationically polymerizable group, and the like. Organic solvents exemplified for specific examples of the solvent (S), and the like can be used as the solvent. In the production of the compound represented by the formula (A-2e-b), reaction temperature is not particularly limited when cyanuric halide is reacted with aromatic amines such as aromatic amines represented by R^{A11}—NH₂, R^{A12}—NH₂, and R^{A13}—NH₂. Typically, reaction temperature is preferably 0° C. or higher and 150° C. or lower.

A content of the photopolymerizable compound (A) in the composition is not particularly limited as long as the desired effects are not impaired. The content of the photopolymerizable compound (A) in the composition is preferably 0.1 parts by mass or more and 50 parts by mass or less, more preferably 0.5 parts by mass or more and 40 parts by mass or less, and particularly preferably 1 part by mass or more and 25 parts by mass or less, when a mass of the composition excluding a mass of the solvent (S) described below is 100 parts by mass.

### <Inorganic Microparticles (B)>

The composition includes inorganic microparticles (B). The material of the inorganic microparticles (B) is not particularly limited as long as it is an inorganic material. The inorganic microparticles (B) are preferably at least one selected from the group consisting of metal oxide microparticles (B1) and metal microparticles (B2). When the composition includes the metal oxide microparticles (B1), a material with high refractive index can be easily formed by using the composition. When the composition includes the metal microparticles (B2), conductivity is imparted to the material formed using the composition, or the optical absorption of light of a specific wavelength in the material formed using the composition is enhanced. Therefore, the compositions including the metal microparticles (B2) are used to form the materials that can be applied to band-pass filters. Type of metal oxide constituting the metal oxide microparticles (B1) is not particularly limited as long as the desired effect is not impaired. Suitable examples of the metal oxide microparticles (B1) include at least one selected from the group consisting of zirconium dioxide microparticles, titanium dioxide microparticles, barium titanate microparticles, cerium dioxide microparticles, and niobium pentoxide microparticles. Type of metal constituting the metal microparticles (B2) is not particularly limited as long as the desired effect is not impaired. Metal constituting the metal microparticles (B) may be a simple substance or an alloy. Suitable examples of the metal microparticles (B2) include gold microparticles and platinum microparticles. Examples of other suitable inorganic microparticles (B) include silicon microparticles (SiNC (silicon nanoparticles)). The composition may include one or combination of two or more selected from these inorganic microparticles (B).

In view of transparency of a material formed by using the composition, and stable dispersibility of the inorganic microparticles (B) in the composition, average particle size of the inorganic microparticles (B) is preferably 500 nm or more, and more preferably 2 nm or more and 100 nm or less.

For the metal oxide microparticles (B1), surfaces thereof are preferably modified with an ethylenically unsaturated double bond. When the surfaces of the metal oxide microparticles (B1) are modified with the ethylenically unsaturated double bond, metal oxide microparticles (B1) are less likely to agglomerate. Therefore, when the surfaces of the metal oxide microparticles (B1) are modified with the ethylenically unsaturated double bond, localization of the metal oxide microparticles (B1) in the material formed by using the composition is easily suppressed.

For example, the metal oxide microparticles (B1) in which the surface thereof is modified with the ethylenically unsaturated double bond can be obtained by reacting a capping agent including ethylenically unsaturated double bond to the surface of the metal oxide microparticles (B1).

A method of binding the capping agent including the ethylenically unsaturated double bond to the surface of the metal oxide microparticles (B1) via a chemical bond such as a covalent bond is not particularly limited. Hydroxy groups usually exist on the surface of the metal oxide microparticles (B1). By reacting hydroxy groups and the reactive groups possessed by the capping agent, the caping agent covalently bonds to the surfaces of the metal oxide microparticles (B1). Suitable examples of the reactive group possessed by the capping agent include a trialkoxysilyl group such as trimethoxysilyl group and triethoxysilyl group; a dialkoxysilyl group such as dimethoxysilyl group and diethoxysilyl group; a monoalkoxysilyl group such as monomethoxysilyl group and monoethoxysilyl group; a trihalosilyl group such as trichlorosilyl group; a dihalosilyl group such as dichlorosilyl group; a monohalosilyl group such as monochlorosilyl group; carboxy group; a halocarbonyl group such as chlorocarbonyl group; hydroxy group; phosphono group (— P(═O)(OH)₂); phosphate group (—O— P(═O)(OH)₂)

The trialkoxysilyl group, the dialkoxysilyl group, the monoalkoxysilyl group, the trihalosilyl group, the dihalosilyl group, and the monohalosilyl group can form siloxane bond with the surfaces of the metal oxide nano particles (B1). Carboxy group and the halocarbonyl group can form a bond represented by (metal oxide-O—CO—) with the surfaces of the metal oxide nano particles (B1). Hydroxy group can form a bond represented by (metal oxide-O—) with the surfaces of the metal oxide nano particles (B1). Phosphono group and phosphate group form a bond represented by (metal oxide-O— P(═O)<) microparticles (B1) with the surfaces of the metal oxide nano particles (B1).

In the capping agent, hydrogen atom and various organic group are exemplified as a group binding to the above reactive group. The organic group may include a hetero atom such as O, N, S, P, B, Si, and a halogen atom. As a group bonding to the above reactive group, for example, an alkyl group that may be straight or branched, and may be interrupted by oxygen atom (—O—), an alkenyl group that may be straight or branched, and may be interrupted by oxygen atom (—O—), an alkynyl group that may be straight or branched, and may be interrupted by oxygen atom (—O—), a cycloalkyl group, an aromatic hydrocarbon group, heterocyclic group, and the like are exemplified. These groups may be substituted with a substituent such as halogen atom, an epoxy group-containing group (e.g. glycidyl group), hydroxy group, amino group, (meth)acrylic group, and isocyanate group. In addition, a number of substituents is not particularly limited.

As a group bonding to the above-described reactive group, a group represented by —(SiR^{b1}R^{b2}—O—)r-(SiR^{b3}R^{b4}—O—)s-R^{b5} is also preferred. R^{b1}, R^{b2}, R^{b3}, and R^{b4} each may be the same or different, and an organic group. Suitable examples of the organic group include an alkyl group such as methyl group and ethyl group; an alkenyl group such as vinyl group and allyl group; an aromatic hydrocarbon group such as phenyl group, naphthyl group, and tolyl group; an epoxy group-containing group such as 3-glycidoxypropy group; (meth)acryloyloxy group and the like. As R^{b5} in the above formula, for example, a terminal group such as —Si(CH₃)₃, —Si(CH₃)₂H, —Si(CH₃)₂(CH═CH₂), and —Si(CH₃)₂(CH₂CH₂CH₂CH₃) is exemplified. r and s in the above formula are each independently an integer of 0 or more and 60 or less. Both r and s in the above formula are never zero.

Suitable specific examples of the capping agent include unsaturated group-containing alkoxysilanes such as vinyltrimethoxysilane, vinyltriethoxysilane, allyltrimethoxysilane, allyltriethoxysilane, 1-hexenyltrimethoxysilane, 1-hexenyltriethoxysilane, 1-octenyltrimethoxysilane, 1-octenyltriethoxysilane, 3-acryloyloxypropyl(trimethoxy)silane, 3-acryloyloxypropyl(triethoxy)silane, 3-methacryloyloxypropyl(trimethoxy)silane, and 3-methacryloyloxypropyl(triethoxy)silane; unsaturated group-containing alcohols such as 2-hydroxyethyl (meth)acrylate, 3-hydroxypropyl (meth)acrylate, allyl alcohol, ethylene glycol monoallyl ether, propylene glycol monoallyl ether, and 3-allyloxypropanol; (meth)acrylic acid; (meth)acrylic acid halides such as (meth)acrylic acid chloride.

When the capping agent is bonded to the surface of the metal oxide microparticles (B1) via a chemical bond such as a covalent bond, an amount of the capping agent is not particularly limited. The capping agent is preferably used in a sufficient amount of to react with almost all hydroxy groups on the surface of the metal oxide microparticles (B1).

A content of the inorganic microparticles (B) is not particularly limited as long as it does not interfere with the object of the present invention. A content of the inorganic microparticles (B) is preferably 5% by mass or more and 95% by mass or less, more preferably 35% by mass or 93% by mass or less, and further preferably 40% by mass or more and 90% by mass or less relative to relative to a mass of the composition excluding a mass of the solvent (S). When a content of the inorganic microparticles (B) in the composition is within the above range, the composition in which the inorganic microparticles (B) are stably dispersed is easily obtained, and the material with the desired effect of use of the inorganic microparticles (B) can be easily formed by using the composition. When the inorganic microparticles (B) are the metal oxide microparticles (B1), a content of the metal oxide microparticles (B1) in the composition is preferably 90% by mass or more, more preferably 90% by mass or more and 98% by mass or less, and further preferably 90% by mass or more and 95% by mass or less relative to a mass of the composition excluding a mass of the solvent (S), since a material with high refractive index is easily formed. When the surfaces of the metal oxide microparticles (B1) are modified with the ethylenically unsaturated double bond-containing group, a mass of the capping agent existing on the surfaces of the metal oxide microparticles (B1) and having the ethylenically unsaturated double bond-containing group is included in a mass of the metal oxide microparticles (B1).

### <Plasticizer (D)>

The composition may include a plasticizer (D). The plasticizer (D) is a component which lowers viscosity of the composition without significantly compromising various properties such as high refractive index of the material formed using the composition.

The plasticizer (D) is preferably a compound represented by following formula (d-1).

R^{d1}—R^{d3}ᵣ—X^{d}—R^{d4}ₛ—R^{d2} (d_1)

In the formula (d-1), R^{d1} and R^{d2} are each independently phenyl group optionally substituted with 1 or more and 5 or less substituents. The substituent is selected from an alkyl group having 1 or more and 4 or less carbon atoms, an alkoxy group having 1 or more and 4 or less carbon atoms, and a halogen atom. R^{d3} and R^{d4} are each independently methylene group or ethane-1,2-diyl group. r and s are each independently 0 or 1. X^{d} is oxygen atom or sulfur atom.

By inclusion of the plasticizer (D) in the composition, viscosity of the composition is lowered without significantly compromising various properties such as high refractive index of the material formed using the composition. From the viewpoint of lowering the viscosity of the composition, the viscosity of the plasticizer (D) measured by an E-type viscometer at 25° C. is preferably 10 cP or less, more preferably 8 cP or less, and further preferably 6 cP or less. From the viewpoint that the plasticizer (D) is less likely to volatilize and an effect of lowering the viscosity of the composition can be easily maintained, the boiling point of the plasticizer (D) under atmospheric pressure is preferably 250° C. or higher, and more preferably 260° C. or higher. Upper limit of the boiling point under atmospheric pressure of the plasticizer (D) is not particularly limited, and may be 300° C. or higher, or 350° C. or higher, for example.

R^{d1} and R^{d2} in the formula (d-1) are each independently a phenyl group optionally substituted with 1 or more and 5 or less substituents. The substituent bonding to the phenyl group is a group selected from an alkyl group having 1 or more and 4 or less carbon atoms, an alkoxy group having 1 or more and 4 or less carbon atoms, and a halogen atom. When the phenyl group has the substituent, a number of substituents is not particularly limited. The number of substituents is 1 or more and 5 or less, preferably 1 or 2, and preferably 1. Since the viscosity of the composition is low, R^{d1} and R^{d2} are respectively preferably unsubstituted phenyl group.

Examples of the alkyl group having 1 or more and 4 or less carbon atoms as the substituent include methyl group, ethyl group, n-propyl group, isopropyl group, n-butyl group, isobutyl group, sec-butyl group, and tert-butyl group. Examples of the alkoxy group having 1 or more and 4 or less carbon atoms as the substituent include methoxy group, ethoxy group, n-propyloxy group, isopropyloxy group, n-butyloxy group, isobutyloxy group, sec-butyloxy group, and tert-butyloxy group. Examples of the halogen atom as the substituent include fluorine atom, chlorine atom, bromine atom, and iodine atom.

R^{d3} and R^{d4} in the formula (d-1) are each independently methylene group or ethane-1,2-diyl group. In addition, r and s are each independently 0 or 1. X^{d} in the formula (d-1) is oxygen atom or sulfur atom.

Suitable specific examples of the compound represented by the formula (d-1) described above include diphenyl ether, diphenyl sulfide, dibenzyl ether, dibenzyl sulfide, diphenethyl ether, and diphenethyl sulfide. Among these, diphenyl sulfide and/or dibenzyl ether are preferred.

From the viewpoint of both adjustment of viscosity and dispersibility of the inorganic microparticles (B), a content of the plasticizer (D) in the composition is preferably 0% by mass or more and 35% by mass or less, and more preferably 5% by mass or more and 15% by mass or less.

### <Nitrogen-Containing Compound (E)>

In order to suppress localization of the inorganic microparticles (B) in the material formed by using the composition, the composition may include an amine compound represented by following formula (e1) and/or an imine compound represented by following formula (e2) as a nitrogen-containing compound (E).

NR^{e1}R^{e2}R^{e3} (e1)

In the formula (e1), R^{e1}, R^{e2}, and R^{e3} are each independently a hydrogen atom or an organic group.

R^{e4}—N═CR^{e5}R^{e6} (e2)

In the formula (e2), R^{e4}, R^{e5}, and R^{e6} are each independently hydrogen atom or an organic group.

When R^{e1}, R^{e2}, R^{e3}, R^{e4}, R^{e5}, and R^{e6} in the formula (e1) and the formula (e2) are the organic group, the organic group can be selected from various organic groups as long as the desired effect is not impaired. As the organic group, a carbon atom-containing group is preferred, and a group consisting of 1 or more carbon atoms, and 1 or more atoms selected from the group consisting of H, O, S, Se, N, B, P, Si and a halogen atom is more preferred. The number of carbon atoms in the carbon atom-containing group is not particularly limited, and is preferably 1 or more and 50 or less, and more preferably 1 or more and 20 or less. Suitable examples of the organic group include an alkyl group, a cycloalkyl group, an optionally substituted phenyl group, an optionally substituted phenylalkyl group, an optionally substituted naphthyl group, an optionally substituted naphthyl alkyl group, an optionally substituted heterocyclyl group, and the like.

A number of carbon atoms in the alkyl group as the organic group is preferably 1 or more and 20 or less, and more preferably 1 or more and 6 or less. Structure of the alkyl group may be straight or branched. Suitable specific examples of the alkyl group include methyl group, ethyl group, n-propyl group, isopropyl group, n-butyl group, isobutyl group, sec-butyl group, tert-butyl group, n-pentyl group, isopentyl group, sec-pentyl group, tert-pentyl group, n-hexyl group, n-heptyl group, n-octyl group, isooctyl group, sec-octyl group, tert-octyl group, n-nonyl group, isononyl group, n-decyl group, isodecyl group, and the like. In addition, the alkyl group may include an ether bond (—O—) in the carbon chain. Examples of the alkyl group having an ether bond in a carbon chain include methoxyethyl group, ethoxyethyl group, methoxyethoxyethyl group, ethoxyethoxyethyl group, propyloxyethoxyethyl group, and methoxypropyl group.

A number of carbon atoms in the cycloalkyl group as the organic group is preferably 3 or more and 10 or less, and more preferably 3 or more and 6 or less. Specific examples of the cycloalkyl group include cyclopropyl group, cyclobutyl group, cyclopentyl group, cyclohexyl group, cycloheptyl group, cyclooctyl group, and the like. A number of carbon atoms in the phenylalkyl group as the organic group is preferably 7 or more and 20 or less, and more preferably 7 or more and 10 or less. A number of carbon atoms in the naphthylalkyl group as the organic group is preferably 11 or more and 20 or less, and more preferably 11 or more and 14 or less. Specific examples of the phenylalkyl group include benzyl group, 2-phenylethyl group, 3-phenylpropyl group, and 4-phenylbutyl group. Specific examples of the naphthylalkyl group include α-naphthylmethyl group, β-naphthylmethyl group, 2-(α-naphthyl)ethyl group, and 2-(β-naphthyl)ethyl group. The phenylalkyl group or the naphthylalkyl group may have a substituent on phenyl group or naphthyl group.

When the organic group is the heterocyclyl group, the heterocyclyl group is the same as when R^{c4} in the formula (c3) is the heterocyclyl group, and the heterocyclyl group may have additional substituents.

The heterocyclyl group as the organic group may be an aliphatic heterocyclic group or an aromatic heterocyclic group. The heterocyclyl group is preferably a heterocyclyl group consisting of 5- or 6-membered single ring including one or more N, S, and O, or a fused ring in which above single rings are fused each other, or a above single ring is fused with a benzene ring. When the heterocyclyl group is a fused ring, the number of rings constituting the fused ring is 3 or less. Examples of the heterocycle constituting the heterocyclyl group include furan, thiophene, pyrrole, oxazole, isoxazole, thiazole, thiadiazole, isothiazole, imidazole, pyrazole, triazole, pyridine, pyrazine, pyrimidine, pyridazine, benzofuran, benzothiophene, indole, isoindole, indolizine, benzoimidazole, benzotriazole, benzoxazole, benzothiazole, carbazole, purine, quinoline, isoquinoline, quinazoline, phthalazine, cinnoline, quinoxaline, piperidine, piperazine, morpholine, piperidine, tetrahydropyran, tetrahydrofuran, and the like.

When phenyl group, naphthyl group, and heterocyclyl group included in the above organic group have substituent, examples of the substituent include an alkyl group having 1 or more and 6 or less carbon atoms, an alkoxy group having 1 or more and 6 or less carbon atoms, a halogenated alkyl group having 1 or more and 6 or less carbon atoms, a halogenated alkoxy group having 1 or more and 6 or less carbon atoms, an aliphatic acyl group having 2 or more and 7 or less carbon atoms, an alkoxycarbonyl group having 2 or more and 7 or less carbon atoms, a saturated aliphatic acyloxy group having 2 or more and 7 or less carbon atoms, a monoalkylamino group having 1 or more and 6 or less carbon atoms, a dialkylamino group having an alkyl group having 1 or more and 6 or less carbon atoms, a benzoyl group, a halogen atom, a nitro group, a cyano group, and the like. When a phenyl group, a naphthyl group, and a heterocyclyl group included in the organic group have one or more substituents, a number of the substituents is not particularly limited, and preferably 1 or more and 4 or less. When a phenyl group, naphthyl group, and a heterocyclyl group included in the organic group have a plurality of substituents, the plurality of substituents may be the same or different.

In the formula (e1), R^{e1}, R^{e2}, and R^{e3} are each independently a hydrogen atom or an organic group, and at least one of R^{e1}, R^{e2}, and R^{e3} is an aromatic group-containing group. In addition, in the formula (e2), R^{e4}, R^{e5}, and R^{e6} are each independently a hydrogen atom or an organic group, and at least one of R^{e4}, R^{e5}, and R^{e6} is an aromatic group-containing group. The aromatic ring in the aromatic ring-containing group may be an aromatic hydrocarbon ring or an aromatic heterocycle. The aromatic ring-containing group is preferably a hydrocarbon group. As the aromatic ring-containing group, an aromatic hydrocarbon group (aryl group) and an aralkyl group are preferred. Examples of the aromatic hydrocarbon group include phenyl group, naphthalen-1-yl group, and naphthalen-2-yl group. Among these aromatic hydrocarbon groups, phenyl group is preferred. Examples of the aralkyl group include benzyl group, 2-phenylethyl group, 3-phenylpropyl group, and 4-phenylbutyl group.

In the formula (e1), at least one of R^{e1}, R^{e2}, and R^{e3} is preferably a group represented by -Ar^{e1}—CH₂—. In addition, in the formula (e2), R^{e4} is preferably a group represented by Ar^{e1}—CH₂—. Ar^{e1} is an optionally substituted aromatic group. The aromatic group as Ar^{e1} may be an aromatic hydrocarbon group, or an aromatic heterocyclic group. The aromatic group as Ar^{e1} is preferably the aromatic hydrocarbon group. Examples of the aromatic hydrocarbon group include phenyl group, naphthalen-1-yl group, and naphthalen-2-yl group. Among these aromatic hydrocarbon groups, phenyl group is preferred. The substituent which the aromatic group as Ar^{e1} may have is the same as the substituent which phenyl group, naphthyl group, and heterocyclyl group may have in case that the organic group as R^{e1}, R^{e2}, R^{e3}, R^{e4}, R^{e5}, and R^{e6} is these groups.

Suitable specific examples of the amine compound represented by the formula (e1) include triphenylamine, N,N-diphenylbenzylamine, N-phenylbenzylamine, tribenzylamine, N,N-dimethylphenylamine, N-methyldiphenylamine, N,N-dimethylbenzylamine, N-methyldibenzylamine, N-methyl-N-benzylphenylamine, N,N-diethylphenylamine, N-ethyldiphenylamine, N,N-diethylbenzylamine, N-ethylbenzylamine, and N-ethyl-N-benzylamine.

Suitable specific examples of the imine compound represented by the formula (e2) include N-benzylphenylmethaneimine, N-benzyldiphenylmethaneimine, N-benzyl-1-phenylethaneimine, and N-benzylpropan-2-imine.

A content of the nitrogen-containing compound (E) is not particularly limited as long as the desired effect is not impaired. The content of the nitrogen-containing compound (E) is preferably 5% by mass or more and 25% by mass or less, and more preferably 7% by mass or more and 20% by mass or less relative to the mass of the photopolymerizable compound (A).

### <Triazine Compound (F)>

For the purpose of forming the cured product with higher refractive index using the composition, the composition may include a compound represented by the following formula (F1) as a triazine compound (F).

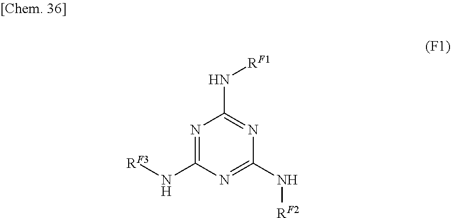

In the formula (F1), R^{F1}, R^{F2}, and R^{F3} are each independently an optionally substituted monocyclic aromatic group or an optionally substituted condensed aromatic group. Provided that, R^{F1}, R^{F2}, and R^{F3} do not include the radically polymerizable group-containing group or the cationically polymerizable group-containing group. When the monocyclic aromatic group or the condensed aromatic group has the substituent, the substituent includes no aromatic ring. Three —NH— groups bonding to the triazine ring respectively bond to aromatic rings in R^{F1}, R^{F2}, and R^{F3}.

The monocyclic aromatic group as R^{F1}, R^{F2}, and R^{F3} may be an aromatic hydrocarbon group or an aromatic heterocyclic group. Examples of the monocyclic aromatic group include phenyl group, pyridinyl group, pyrimidinyl group, pyradinyl group, pyridazinyl group, furanyl group, thienyl group, oxazolyl group, thiazolyl group, and the like.

Examples of the substituted which the monocyclic aromatic group may have include halogen atom, hydroxy group, mercapto group, cyano group, nitro group, and a monovalent organic group. Provided that, the monovalent organic group includes no aromatic ring. Examples of halogen atom as the substituent include fluorine atom, chlorine atom, bromine atom, and iodine atom. Examples of the monovalent organic group include an alkyl group, an alkoxy group, an alkoxyalkyl group, an aliphatic acryl group, an aliphatic acyloxy group, an alkoxycarbonyl group, an alkylthio group, an aliphatic acylthio group, and the like.

A number of carbon atoms in the monovalent organic group as the substituent is not particularly limited as long as the desired effect is not impaired. For example, the number of carbon atoms in the monovalent organic group as the substituent is preferably 1 or more and 20 or less, more preferably 1 or more and 12 or less, and further preferably 1 or more and 8 or less. In the alkoxyalkyl group, the aliphatic acyl group, the aliphatic acyloxy group, the alkoxycarbonyl group, the alkoxyalkylthio group, and the aliphatic acylthio group, lower limit of the number of carbon atoms is 2.

Suitable specific examples of the alkyl group as the substituent include methyl group, ethyl group, n-propyl group, isopropyl group, n-butyl group, isobutyl group, sec-butyl group, tert-butyl group, n-pentyl group, n-hexyl group, n-heptyl group, and n-octyl group.

Suitable specific examples of the alkoxy group as the substituent include methoxy group, ethoxy group, n-propyloxy group, isopropyl oxy group, n-butyloxy group, isobutyloxy group, sec-butyloxy group, tert-butyloxy group, n-pentyloxy group, n-hexyloxy group, n-heptyloxy group, and n-octyloxy group.

Suitable specific examples of the alkoxyalkyl group include methoxymethyl group, ethoxymethyl group, n-propyloxymethyl group, n-butyloxymethyl group, 2-methoxyethyl group, 2-ethoxyethyl group, 2-n-propyloxyethyl group, 2-n-butyloxyethyl group, 3-methoxy-n-propyl group, 3-ethoxy-n-propyl group, 3-n-propyloxy-n-propyl group, 3-n-butyloxy-n-propyl group, 4-methoxy-n-bnutyl group, 4-ethoxy-n-butyl group, 4-n-propyloxy-n-butyl group, and 4-n-butyloxy-n-butyl group.

Suitable specific examples of the aliphatic acyl group include acetyl group, propionyl group, butanoyl group, pentanoyl group, hexanoyl group, heptanoyl group, and octanoyl group.

Suitable specific examples of the aliphatic acyloxy group as the substituent include acetoxy group, propionyloxy group, butanoyloxy group, pentanoyloxy group, hexanoyloxy group, heptanoyloxy group, and octanoyloxy group.

Suitable specific examples of the alkoxycarbonyl group as the substituent include methoxycarbonyl group, ethoxycarbonyl group, n-propyloxycarbonyl group, isopropyloxycarbonyl group, n-butyloxycarbonyl group, isobutyloxycarbonyl group, sec-butyloxycarbonyl group, tert-butyloxycarbonyl group, n-pentyloxycarbonyl group, n-hexyloxycarbonyl group, n-heptyloxycarbonyl group, and n-octyloxycarbonyl group.

Suitable specific examples of the alkylthio group as the substituent include methylthio group, ethylthio group, n-propylthio group, isopropylthio group, n-butylthio group, isobutylthio group, sec-butylthio group, tert-butylthio group, n-pentylthio group, n-hexylthio group, n-heptylthio group, and n-octylthio group.

Suitable specific examples of the aliphatic acylthio group as the substituent include acetylthio group, propionylthio group, butanoylthio group, pentanoylthio group, hexanoylthio group, heptanoylthio group, and ocatnoylthio group.

When the monocyclic aromatic group has the substituent, a number of the substituent is not particularly limited as long as the desired effect is not impaired. When the monocyclic aromatic group has the substituent, the number of the substituent is preferably 1 or more and 4 or less, more preferably 1 or 2, and particularly preferably 1. When the monocyclic aromatic group has a plurality of the substituents, the plurality of the substituents may be different from each other.

Examples of the above-described optionally substituted monocyclic aromatic group include phenyl group, 4-cyanophenyl group, 3-cyanophenyl group, 2-cyanophenyl group, 2,3-dicyanophenyl group, 2,4-dicyanophenyl group, 2,5-dicyanophenyl group, 2,6-dicyanophenyl group, 3,4-dicyanophenyl group, 3,5-dicyanopohenyl group, 4-nitrophenyl group, 3-nitrophenyl group, 2-nitrophenyl group, 4-chlorophenyl group, 3-chlorophenyl group, 2-chlorophenyl group, 4-bromophenyl group, 3-bromophenyl group, 2-bromophenyl group, 4-iodophenyl group, 3-iodophenyl group, 2-iodophenyl group, 4-methoxyphenyl group, 3-methoxyphenyl group, 2-methoxyphenyl group, 4-methylphenyl group, 3-methylphenyl group, and 2-methylphenyl group.

Among these groups, phenyl group, 4-cyanophenyl group, 3-cyanophenyl group, 2-cyanophenyl group, 4-nitrophenyl group, 3-nitrophenyl group, and 2-nitrophenyl group are preferred, and phenyl group, and 4-cyanophenyl group are more preferred.

The condensed aromatic group as R^{F1}, R^{F2}, and R^{F3} is a group in which one hydrogen atom is excluded from the fused polycycle in which two or more aromatic monocycles are fused. A number of aromatic monocycles constituting the condensed aromatic group is not particularly limited. The number of aromatic monocycles constituting the condensed aromatic group is preferably 2 or 3, and more preferably 2. That is, the condensed aromatic group is preferably a bicyclic condensed aromatic group or a tricyclic condensed aromatic group, and more preferably a bicyclic condensed aromatic group. The condensed aromatic group may be an aromatic hydrocarbon group, or an aromatic heterocyclic group.

For example, examples of the bicyclic condensed aromatic group include naphthalen-1-yl group, naphthalen-2-yl group, quinolin-2-yl group, quinolin-3-yl group, quinolin-4-yl group, quinolin-5-yl group, quinolin-6-yl group, quinolin-7-yl group, quinolin-8-yl group, isoquinolin-1-yl group, isoquinolin-3-yl group, isoquinolin-4-yl group, isoquinolin-5-yl group, isoquinolin-6-yl group, isoquinolin-7-yl group, isoquinolin-8-yl group, benzoxazol-2-yl group, benzoxazol-4-yl group, benzoxazol-5-yl group, benzoxazol-6-yl group, benzoxazol-7-yl group, benzothiazol-2-yl group, benzothiazol-4-yl group, benzothiazol-5-yl group, benzothiazol-6-yl group, benzothiazol-7-yl group, and the like.

For example, examples of the tricyclic condensed aromatic group include anthracen-1-yl group, anthracen-2-yl group, anthracen-9-yl group, phenanthren-1-yl group, phenanthren-2-yl group, phenanthren-3-yl group, phenanthren-4-yl group, phenanthren-9-yl group, acridin-1-yl group, acridin-2-yl group, acridin-3-yl group, acridin-4-yl group, and acridin-9-yl group.

The substituent which the polycyclic condensed aromatic group such as the bicyclic condensed aromatic group and the tricyclic condensed aromatic group may have is the same as the substituent which monocyclic aromatic group may have.

As the above-described optionally substituted condensed aromatic group, naphthalen-1-yl group, naphthalen-2-yl group, quinolin-2-yl group, quinolin-3-yl group, quinolin-4-yl group, quinolin-5-yl group, quinolin-6-yl group, quinolin-7-yl group, quinolin-8-yl group, benzothiazol-2-yl group, and 2-mercaptobenzothiazol-6-yl group are preferred.

Among these groups, naphthalen-1-yl group, quinolin-3-yl group, quinolin-4-yl group, and 2-mmercaptobenzothiazol-6-yl group are preferred, and naphthalen-1-yl group is more preferred.

Among the above-described compounds represented by the formula (F1), a compound in which 1 or more of R^{F1}, R^{F2}, and R^{F3} is the optionally substituted naphthyl group, and 1 or more of R^{F1}, R^{F2}, and R^{F3} is 4-cyanophenyl group or benzothiazolyl group is preferred, since a refractive index, a surface appearance, and a heat resistance of the material formed by using the composition are good in balance. As the optionally substituted naphthyl group, naphthalen-1-yl group is preferred.

Suitable examples of the compound represented by the formula (F1) include the following compounds.

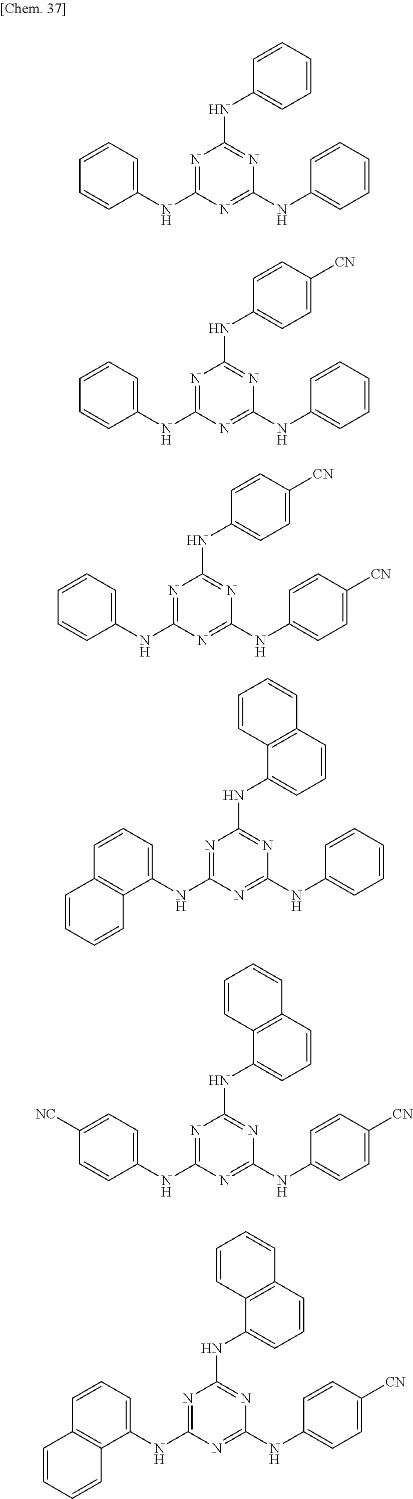

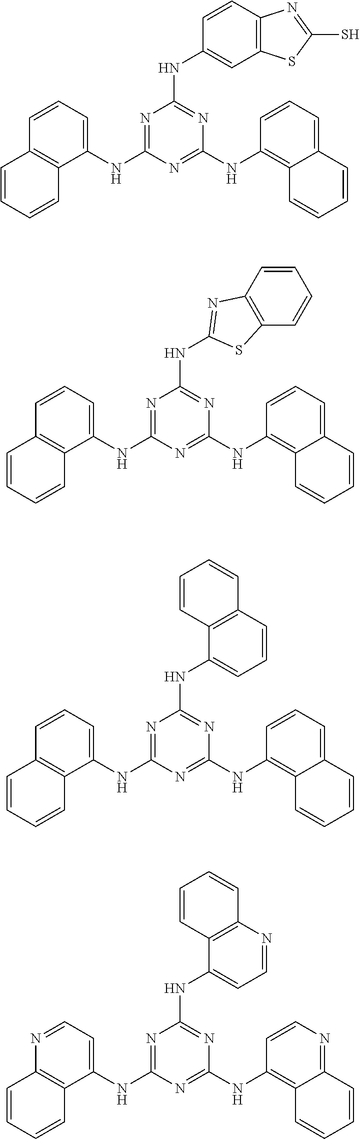 [SMILES: Sc1nc2ccc(Nc3nc(Nc4cccc5ccccc45)nc(Nc4cccc5ccccc45)n3)cc2s1 ; c1ccc2c(Nc3nc(Nc4nc5ccccc5s4)nc(Nc4cccc5ccccc45)n3)cccc2c1 ; c1ccc2c(Nc3nc(Nc4cccc5ccccc45)nc(Nc4cccc5ccccc45)n3)cccc2c1 ; c1ccc2c(Nc3nc(Nc4ccnc5ccccc45)nc(Nc4ccnc5ccccc45)n3)ccnc2c1]

Production method of the compound represented by the formula (F1) is not particularly limited. Typically, the compound represented by the formula (F1) can be prepared by reacting cyanuric halide such as cyanuric chloride with aromatic amines represented by R^{F1}—NH₂, R^{F2}—NH₂, and R^{F3}—NH₂. These multiple amines may react with the cyanuric halide simultaneously or sequentially, and preferably sequentially react with the cyanuric halide.

The compound represented by the formula (F1) is usually synthesized in an organic solvent. This organic solvent is not particularly limited as long as the solvent is an inactive solvent which does not react with cyanuric halide, aromatic amine, radically polymerizable group, cationically polymerizable group, and the like. As the solvent, solvents exemplified as specific examples of the solvent (S) described below can be used. In the production of the compound represented by the formula (F1), reaction temperature is not particularly limited when cyanuric halide is reacted with aromatic amines such as aromatic amines represented by R^{F1}—NH₂, R^{F2}—NH₂, and R^{F13}—NH₂. Typically, reaction temperature is preferably 0° C. or higher and 150° C. or lower.

An amount of the triazine compound (F) in the composition is not particularly limited as long as the desired effect is impaired. The amount of the triazine compound (F) in the composition is preferably 0.1 parts by mass or more and 30 parts by mass or less, 0.3 parts by mass or more and 20 parts by mass or less, and further preferably 0.5 parts by mass or more and 15 parts by mass or less, when a mass of the composition excluding a mass of the solvent (S) described below is 100 parts by mass.

### <Solvent (S)>

The composition may include a solvent (S) for purposes of adjusting applicability and the like. Type of the solvent (S) is not particularly limited as long as the desired effect is not impaired.

For example, suitable examples of the solvent (S) include (poly)alkyleneglycol monoalkyl ethers such as ethylene glycol monomethyl ether, ethylene glycol monoethyl ether, ethylene glycol mono-n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether (HO—CH₂CH₂CH₂—O—CH₃), propylene glycol monomethyl ether (HO—C(CH₃)HCH₂—O—CH₃ or H₃C—O—C(CH₃)HCH₂—OH), propylene glycol monoethyl ether (HO—CH₂CH₂CH₂—O—CH₂CH₃), propylene glycol monomethyl ether (HO—C(CH₃)HCH₂—O—CH₂CH₃ or H₃CH₂C—O—C(CH₃)HCH₂—OH), propylene glycol mono-n-propyl ether (HO—CH₂CH₂CH₂—O—CH₂CH₂CH₃), propylene glycol mono-n-propyl ether (HO—C(CH₃)HCH₂—O—CH₂CH₂CH₃ or H₃CH₂CH₂C—O—C(CH₃)HCH₂—OH), propylene glycol mono-n-butyl ether (HO—CH₂CH₂CH₂—O—CH₂CH₂CH₃), propylene glycol mono-n-butyl ether (HO—C(CH₃)HCH₂—O—CH₂CH₂CH₂CH₃ or H₃CH₂CH₂CH₂C—O—C(CH₃)HCH₂—OH), dipropylene glycol monoethyl ether (HO—(CH₂CH₂CH₂—O)₂—CH₃), dipropylene glycol monomethyl ether (HO—(C(CH₃)HCH₂—O)₂—CH₃ or H₃C—O—(C(CH₃)HCH₂—O)₂—H), dipropylene glycol monoethyl ether (HO—(CH₂CH₂CH₂—O)₂—CH₂CH₃), dipropylene glycol monoethyl ether (HO—(C(CH₃)HCH₂—O)₂—CH₂CH₃ or H₃CH₂C—O—(C(CH₃)HCH₂—O)₂—H), dipropylene glycol mono-n-propyl ether (HO—(CH₂CH₂CH₂—O)₂—CH₂CH₂CH₃ or H₃CH₂CH₂C—O—(CH₂CH₂CH₂—O)₂—H), dipropylene glycol mono-n-propyl ether (HO—(C(CH₃)HCH₂—O)₂—CH₂CH₂CH₃ or H₃CH₂CH₂C—O—(C(CH₃)HCH₂—O)₂—H), dipropylene glycol mono-n-butyl ether (HO—(CH₂CH₂CH₂—O)₂—CH₂CH₂CH₂CH₃ or H₃CH₂CH₂CH₂C—O—(CH₂CH₂CH₂—O)₂—H), dipropylene glycol mono-n-butyl ether (HO—(C(CH₃)HCH₂—O)₂—CH₂CH₂CH₂CH₃ or H₃CH₂CH₂CH₂C—O—(C(CH₃)HCH₂—O)₂—H), tripropylene glycol monomethyl ether (HO—(CH₂CH₂CH₂—O)₃-GE₂CH₃), tripropylene glycol monomethyl ether (HO—(C(CH₃)HCH₂—O)₃—CH₃ or H₃C—O—(C(CH₃)HCH₂—O)₃—H), tripropylene glycol monoethyl ether (HO—(CH₂CH₂CH₂—O)₃—CH₂CH₃), and tripropylene glycol monoethyl ether (HO—(C(CH₃)HCH₂—O)₃—CH₂CH₃ or H₃CH₂C—O—(C(CH₃)HCH₂—O)₃—H); (poly)alkylene glycol monoalkyl ether acetates such as ethylene glycol monomethyl ether acetate, ethylene glycol monoethyl ether acetate, diethylene glycol monomethyl ether acetate, diethylene glycol monoethyl ether acetate, propylene glycol monomethyl ether acetate, and propylene glycol monoethyl ether acetate; other ethers such as ethylene glycol dimethyl ether, ethylene glycol diethyl ether, ethylene glycol methyl ethyl ether, diethylene glycol dimethyl ether, diethylene glycol methyl ethyl ether, diethylene glycol diethyl ether, propylene glycol dimethyl ether, propylene glycol diethyl ether, propylene glycol methyl ethyl ether, dipropylene glycol dimethyl ether, dipropylene glycol methyl ethyl ether, dipropylene glycol diethyl ether, and tetrahydrofuran; ketones such as methyl ethyl ketone, cyclohexanone, 2-hepotanone, and 3-heptanone; lactic acid alkyl esters such as methyl 2-hydroxypropionate, and ethyl 2-hydroxypropionate; other esters such as ethyl 2-hydroxy-2-methylpropionate, methyl 3-methoxypropionate, ethyl 3-methoxypropionate, methyl-3-ethoxypropionate, ethyl 3-ethoxypropionate, ethyl ethoxyacetate, ethyl hydroxyacetate, methyl 2-hydroxy-3-methylbutanoate, 3-nethoxybutyl acetate, 3-methyl-3-methoxybutyl acetate, 3-methyl-3-methoxybutyl propionate, ethyl acetate, n-propyl acetate, isopropyl acetate, n-butyl acetate, isobutyl acetate, n-pentyl formate, isopentyl acetate, n-butyl propionate, ethyl butanoate, n-propyl butanoate, isopropyl butanoate, n-butyl butanoate, methyl pyruvate, ethyl pyruvate, n-propyl pyruvate, methyl acetoacetate, ethyl acetoacetate, and ethyl 2-oxabutanoate; aromatic hydrocarbons such as toluene and xylene, amides such as N-methylpyrrolidone, N,N-dimethylformamide, and N,N-dimethylacetamide, and the (poly)alkylene glycol monoalkyl ether acetates are preferred.

From the viewpoint that application by inkjet method can be performed well, the solvent (S) preferably includes a solvent having boiling point of 140° C. or higher under atmospheric pressure, and more preferably includes a high boiling point solvent (Si) having boiling point of 170° C. or higher under atmospheric pressure.

Specific examples of the solvent having boiling point of 140° C. or higher under atmospheric pressure include ethylene glycol mono-n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether (HO—CH₂CH₂CH₂—O—CH₃), propylene glycol monoethyl ether (HO—CH₂CH₂CH₂—O—CH₂CH₃), propylene glycol mono-n-propyl ether (HO—CH₂CH₂CH₂—O—CH₂CH₂CH₃), propylene glycol mono-n-propyl ether (HO—C(CH₃)HCH₂—O—CH₂CH₂CH₃ or H₃CH₂CH₂C—O—C(CH₃)HCH₂—OH), propylene glycol mono-n-butyl ether (HO—CH₂CH₂CH₂—O—CH₂CH₂CH₂CH₃), propylene glycol mono-n-butyl ether (HO—C(CH₃)HCH₂—O—CH₂CH₂CH₂CH₃ or H₃CH₂CH₂CH₂C—O—C(CH₃)HCH₂—OH), dipropylene glycol monoethyl ether (HO—(CH₂CH₂CH₂—O)₂—CH₃), dipropylene glycol monomethyl ether (HO—(C(CH₃)HCH₂—O)₂—CH₃ or H₃C—O—(C(CH₃)HCH₂—O)₂—H), dipropylene glycol monoethyl ether (HO—(CH₂CH₂CH₂—O)₂—CH₂CH₃), dipropylene glycol monoethyl ether (HO—(C(CH₃)HCH₂—O)₂—CH₂CH₃ or H₃CH₂C—O—(C(CH₃)HCH₂—O)₂—H), dipropylene glycol mono-n-propyl ether (HO—(CH₂CH₂CH₂—O)₂—CH₂CH₂CH₃ or H₃CH₂CH₂C—O—(CH₂CH₂CH₂—O)₂—H), dipropylene glycol mono-n-propyl ether (HO—(C(CH₃)HCH₂—O)₂—CH₂CH₂CH₃ or H₃CH₂CH₂C—O—(C(CH₃)HCH₂—O)₂—H), dipropylene glycol mono-n-butyl ether (HO—(CH₂CH₂CH₂—O)₂—CH₂CH₂CH₂CH₃ or H₃CH₂CH₂CH₂C—O—(CH₂CH₂CH₂—O)₂—H), dipropylene glycol mono-n-butyl ether (HO—(C(CH₃)HCH₂—O)₂—CH₂CH₂CH₂CH₃ or H₃CH₂CH₂CH₂C—O—(C(CH₃)HCH₂—O)₂—H), tripropylene glycol monomethyl ether (HO—(CH₂CH₂CH₂—O)₃-GE₂CH₃), tripropylene glycol monomethyl ether (HO—(C(CH₃)HCH₂—O)₃—CH₃ or H₃C—O—(C(CH₃)HCH₂—O)₃—H), tripropylene glycol monoethyl ether (HO—(CH₂CH₂CH₂—O)₃—CH₂CH₃), tripropylene glycol monoethyl ether (HO—(C(CH₃)HCH₂—O)₃—CH₂CH₃ or H₃CH₂C—O—(C(CH₃)HCH₂—O)₃—H), diethylene glycol monomethyl ether acetate, diethylene glycol monoethyl ether acetate, diethylene glycol dimethyl ether, cyclohexanone, 2-hepotanone, ethyl 2-hydroxypropionate, ethyl 2-hydroxy-2-methylpropionate, ethyl 3-methoxypropionate, ethyl 3-methoxypropionate, ethyl 3-ethoxypropionate, ethyl ethoxyacetate, 3-nethoxybutyl acetate, 3-methyl-3-methoxybutyl acetate, 3-methyl-3-methoxybutyl propionate, n-butyl butanoate, methyl acetoacetate, ethyl acetoacetate, ethyl 2-oxabutanoate, N-methylpyrrolidone, N,N-dimethylformamide, and N,N-dimethylacetamide.

Specific examples of the high boiling point solvent (Si) include ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol mono-n-butyl ether (HO—CH₂CH₂CH₂—O—CH₂CH₂CH₂CH₃), propylene glycol mono-n-butyl ether (HO—C(CH₃)HCH₂—O—CH₂CH₂CH₂CH₃ or H₃CH₂CH₂CH₂C—O—C(CH₃)HCH₂—OH), dipropylene glycol monoethyl ether (HO—(CH₂CH₂CH₂—O)₂—CH₃), dipropylene glycol monomethyl ether (HO—(C(CH₃)HCH₂—O)₂—CH₃ or H₃C—O—(C(CH₃)HCH₂—O)₂—H), dipropylene glycol monoethyl ether (HO—(CH₂CH₂CH₂—O)₂—CH₂CH₃), dipropylene glycol monoethyl ether (HO—(C(CH₃)HCH₂—O)₂—CH₂CH₃ or H₃CH₂C—O—(C(CH₃)HCH₂—O)₂—H), dipropylene glycol mono-n-propyl ether (HO—(CH₂CH₂CH₂—O)₂—CH₂CH₂CH₃ or H₃CH₂CH₂C—O—(CH₂CH₂CH₂—O)₂—H), dipropylene glycol mono-n-propyl ether (HO—(C(CH₃)HCH₂—O)₂—CH₂CH₂CH₃ or H₃CH₂CH₂C—O—(C(CH₃)HCH₂—O)₂—H), dipropylene glycol mono-n-butyl ether (HO—(CH₂CH₂CH₂—O)₂—CH₂CH₂CH₂CH₃ or H₃CH₂CH₂CH₂C—O—(CH₂CH₂CH₂—O)₂—H), dipropylene glycol mono-n-butyl ether (HO—(C(CH₃)HCH₂—O)₂—CH₂CH₂CH₂CH₃ or H₃CH₂CH₂CH₂C—O—(C(CH₃)HCH₂—O)₂—H), tripropylene glycol monomethyl ether (HO—(CH₂CH₂CH₂—O)₃—CH₂CH₃), tripropylene glycol monomethyl ether (HO—(C(CH₃)HCH₂—O)₃—CH₃ or H₃C—O—(C(CH₃)HCH₂—O)₃—H), tripropylene glycol monoethyl ether (HO—(CH₂CH₂CH₂—O)₃—CH₂CH₃), tripropylene glycol monoethyl ether (HO—(C(CH₃)HCH₂—O)₃—CH₂CH₃ or H₃CH₂C—O—(C(CH₃)HCH₂—O)₃—H), diethylene glycol monomethyl ether acetate, diethylene glycol monoethyl ether acetate, diethylene glycol methyl ethyl ether, diethylene glycol diethyl ether, dipropylene glycol dimethyl ether, dipropylene glycol methyl ethyl ether, dipropylene glycol diethyl ether, ethyl hydroxy acetate, 3-methoxybutyl acetate, 3-methyl-3-methoxybutyl acetate, 3-methyl-3-methoxybutyl propionate, methyl acetoacetate, ethyl acetoacetate, ethyl 2-oxobutanoate, and N-methyhlpyrrolidone.

In view of easily obtaining the desired effect, a ratio of a mass of the solvent having boiling point of 140° C. or higher or the high boiling point solvent (S1) having boiling point of 170° C. or higher is preferably 20% by mass or more, more preferably 30% by mass or more, further preferably 50% by mass or more, even more preferably 70% by mass or more, particularly preferably 90% by mass or more, and most preferably 100% by mass relative to the mass of the solvent (S).

A content of the solvent (S) is in an amount such that the concentration of components other than the solvent (S) in the composition is preferably 1% by mass or more and 99% by mass or less, more preferably 5% by mass or more and 50% by mass or less, and further preferably 10% by mass or more and 30% by mass or less.

### <Other Component>

The composition may include various additives as needed, as other components other than the components described above. Examples of the additive include a sensitizer, a curing accelerator, a filler, a dispersant, an adhesion promoter such as a silane coupling agent, an antioxidant, an antiaggregant agent, a thermal polymerization inhibitor, a defoaming agent, and a surfactant. The amount of these additives used is appropriately determined in consideration of the amount of these additives usually used in the composition.

### <<Photosensitive Composition>>

A photosensitive composition includes a photopolymerizable compound (A), inorganic microparticles (B), and an initiator (C). The initiator (C) is a component that cures the photosensitive composition. Suitable embodiments and amounts of the photopolymerizable compound (A) and inorganic microparticles (B) are respectively as described above for the above composition.

The photosensitive composition may further include at least one component selected from the group consisting of the plasticizer (D), the nitrogen-containing compound (E), the triazine compound (F), the solvent (S), and other components, and respectively described above for the composition. Suitable embodiments and amounts of these components are respectively as described above for the above composition.

### <Initiator (C)>

In order to cure the photopolymerizable compound (A), the photosensitive composition includes an initiator (C). When the photopolymerizable compound (A) has the radically polymerizable group, a radical polymerization initiator (Cl) is used as the initiator (C). When the photopolymerizable compound (A) has the cationically polymerizable group, a cationically polymerization initiator (C2) is used as the initiator (C). Since regioselective curing of the photosensitive composition is capable, and there is no concern about thermal degradation, volatilization, or sublimation of the components of the photosensitive composition, the photo initiator is preferred as the initiator (C). The initiator (C) is not particularly limited and various polymerization initiator conventionally known can be used.

Specific examples of the photo radical polymerization initiator the radical polymerization initiator (Cl) include 1-hydroxycyclohexyl phenyl ketone, 2-hydroxy-2-methyl-1-phenylpropan-1-one, 1-[4-(2-hydroxyethoxy)phenyl]-2-hydroxy-2-methyl-1-propan-1-one, 1-(4-isopropylphenyl)-2-hydroxy-2-methylpropan-1-one, 1-(4-dodecylphenyl)-2-hydroxy-2-methylpropan-1-one, 2,2-dimethoxy-1,2-diphenylethan-1-one, bis(4-dimethylaminophenyl) ketone, 2-methyl-1-[4-(methylthio)phenyl]-2-morpholinopropan-1-one, 2-benzyl-2-dimethylamino-1-(4-morpholinophenyl)-butan-1-one, O-acetyl-1-[6-(2-methylbenzoyl)-9-ethyl-9H-carbazol-3-yl]ethanone oxime, (9-ethyl-6-nitro-9H-carbazol-3-yl) [4-(2-methoxy-1-methylethoxy)-2-methylphenyl]methanon 0-acetyloxime, 1,2-octanedione, 2-(benzoyloxyimino)-1-[4-(phenylthio)phenyl]-1-octanone, 2,4,6-trimethylbenzoyldiphenylphosphine oxide, 4-benzoyl-4′-methyldimethyl sulfide, 4-dimethylaminobenzoic acid, methyl 4-dimethylaminobenzoate, ethyl 4-dimethylaminobenzoate, butyl 4-dimethylaminobenzoate, 4-dimethylamino-2-ethylhexylbenzoic acid, 4-dimethylamino-2-isoamylbenzoic acid, benzyl-β-methoxyethyl acetal, benzyl dimethyl ketal, 1-phenyl-1,2-propanedion-2-(0-ethoxycarbonyl) oxime, methyl o-benzoylbenzoate, 2,4-diethylthioxanthone, 2-chlorothioxanthone, 2,4-dimethylthioxanthone, 1-chloro-4-propoxythioxanthone, thioxanthene, 2-chlorothioxanthene, 2,4-diethylthioxanthene, 2-methylthioxanthene, 2-isopropylthioxanthene, 2-ethylanthraquinone, octamethylanthraquinone, 1,2-benzanthraquinone, 2,3-diphenylanthraquinone, azobisisobutyronitrile, benzoyl peroxide, cumene hydroperoxide, 2-mercaptobenzimidazole, 2-mercaptobenzoxazole, 2-mercaptobenzothiazole, 2-(o-chlorophenyl)-4,5-di(m-methoxyphenyl)-imidazolyl dimer, benzophenone, 2-chlorobenzophenone, p,p′-bisdimethylaminobenzophenone, 4,4′-bisdiethylaminobenzophenone, 4,4′-dichlorobenzophenone, 3,3-dimethyl-4-methoxybenzophenone, benzil, benzoin, benzoin methyl ether, benzoin ethyl ether, benzoin isopropyl ether, benzoin-n-butyl ether, benzoin isobutyl ether, benzoin butyl ether, acetophenone, 2,2-diethoxyacetophenone, p-dimethylacetophenone, p-dimethylaminopropiophenone, dichloroacetophenone, trichloroacetophenone, p-tert-butylacetophenone, p-dimethylaminoacetophenone, p-tert-butyltrichloroacetophenone, p-tert-butyldichloroacetophenone, α,α-dichloro-4-phenoxyacetophenone, thioxanthone, 2-methylthioxanthone, 2-isopropylthioxanthone, dibenzosuberone, pentyl 4-dimethylaminobenzoate, 9-phenylacridine, 1,7-bis-(9-acridinyl)heptane, 1,5-bis-(9-acridinyl)pentane, 1,3-bis-(9-acridinyl)propane, p-methoxytriazine, 2,4,6-tris(trichloromethyl)-s-triazine, 2-methyl-4,6-bis(trichloromethyl)-s-triazine, 2-[2-(5-methylfuran-2-yl)ethenyl]-4,6-bis(trichloromethyl)-s-triazine, 2-[2-(furan-2-yl)ethenyl]-4,6-bis(trichloromethyl)-s-triazine, 2-[2-(4-diethylamino-2-methylphenyl)ethenyl]-4,6-bis(trichloromethyl)-s-triazine, 2-[2-(3,4-dimethoxyphenyl)ethenyl]-4,6-bis(trichloromethyl)-s-triazine, 2-(4-methoxyphenyl)-4,6-bis(trichloromethyl)-s-triazine, 2-(4-ethoxystyryl)-4,6-bis(trichloromethyl)-s-triazine, 2-(4-n-butoxyphenyl)-4,6-bis(trichloromethyl)-s-triazine, 2,4-bis-trichloromethyl-6-(3-bromo-4-methoxy)phenyl-s-triazine, 2,4-bis-trichloromethyl-6-(2-bromo-4-methoxy)phenyl-s-triazine, 2,4-bis-trichloromethyl-6-(3-bromo-4-methoxy)styrylphenyl-s-triazine, 2,4-bis-trichloromethyl-6-(2-bromo-4-methoxy)styrylphenyl-s-triazine, and the like. These photo radical polymerization initiators may be used either individually or in combination of two or more.

Among photo radical polymerization initiators, an oxime ester compound is preferred from the viewpoint of sensitivity of the photosensitive composition. A compound including the partial structure represented by the formula (c1) is preferred as the oxime ester compound.

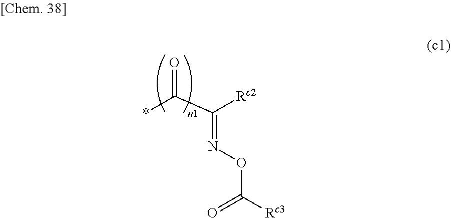

In the formula (c1), n1 is 0 or 1. R^{c2} is a monovalent organic group. R^{c3} is hydrogen atom, an optionally substituted aliphatic hydrocarbon group having 1 or more and 20 or less carbon atoms, or an optionally substituted aryl group. * is a bond.

The compound including the partial structure represented by the formula (c1) preferably has a carbazole skeleton, a fluorene skeleton, a diphenyl ether skeleton, or a phenyl sulfide skeleton. The compound including the partial structure represented by the formula (c1) preferably has 1 or 2 partial structures represented by the formula (c1).

Examples of the compound including the partial structure represented by the formula (c1) includes a compound represented by the following formula (c2).

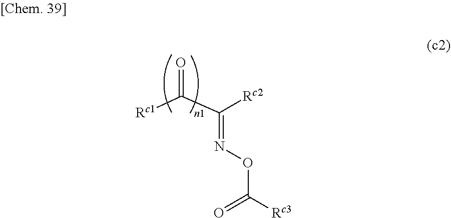

In the formula (c2), R^{c1} is a group represented by following formula (c3), (c4) or (c5). n1 is 0 or 1. R^{c2} is a monovalent organic group. R^{c3} is a hydrogen atom, an optionally substituted aliphatic hydrocarbon group having 1 or more and 20 or less carbon atoms, or an optionally substituted aryl group.

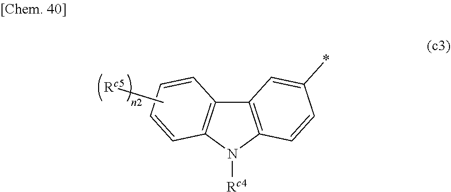

In the formula (c3), R^{c4} and R^{c5} are each independently a monovalent organic group. n2 is an integer of 0 or more and 3 or less. When n2 is 2 or 3, plural R^{c5}s may be the same as or different each other, and plural R^{c5}s may be combined to each other to form a ring. * is a bond.

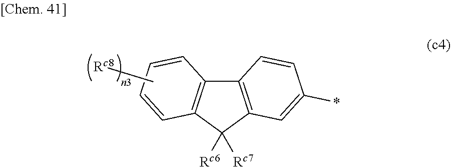

In the formula (c4), R^{c6} and R^{c7} are each independently an optionally substituted chain alkyl group, an optionally substituted chain alkoxy group, an optionally substituted cyclic organic group, or hydrogen atom. R^{c6} and R^{c7} may be combined to each other to form a ring. R^{c7} and a benzene ring in the fluorene skeleton may be combined to each other to form a ring. R^{c8} is a nitro group or a monovalent organic group. n3 is an integer of 0 or more and 4 or less. * is a bond.

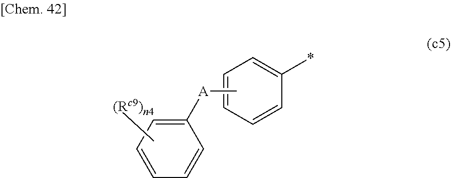

In the formula (C5), R^{c9} is a monovalent organic group, a halogen atom, nitro group, or cyano group. A is sulfur atom or oxygen atom. n4 is an integer of 0 or more and 4 or less. * is a bond.

In the formula (c3), R^{c4} is a monovalent organic group. R^{c4} can be selected from various kinds of organic groups as long as it does not interfere with the object of the present invention. As the organic group, a carbon atom-containing group is preferred, and a group consisting of 1 or more carbon atoms, and 1 or more atoms selected from the group consisting of H, O, S, Se, N, B, P, Si and a halogen atom is more preferred. The number of carbon atoms in the carbon atom-containing group is not particularly limited, and is preferably 1 or more and 50 or less, and more preferably 1 or more and 20 or less. Suitable examples of R^{c4} include an optionally substituted alkyl group having 1 or more and 20 or less carbon atoms, an optionally substituted cycloalkyl group having 3 or more an 20 or less carbon atoms, an optionally substituted saturated aliphatic acyl group having 2 or more and 20 or less carbon atoms, an optionally substituted alkoxycarbonyl group having 2 or more and 20 or less carbon atoms, an optionally substituted phenyl group, an optionally substituted benzoyl group, an optionally substituted phenoxycarbonyl group, an optionally substituted phenylalkyl group having 7 or more and 20 or less carbon atoms, an optionally substituted naphthoyl group, an optionally substituted naphthoxycarbonyl group, an optionally substituted naphthylalkyl group having 11 or more and 20 or less carbon atoms, an optionally substituted heterocyclyl group, an optionally substituted heterocyclylcarbonyl group, and the like.

The alkyl group having 1 or more and 20 or less carbon atoms is preferred as R^{c4}. The alkyl group may be linear or branched. The number of carbon atoms in the alkyl group as R^{c4} is preferably 2 or more, more preferably 5 or more, and particularly preferably 7 or more, from the viewpoint of good solubility of the compound represented by the formula (c3) in the photosensitive composition. From the viewpoint of good compatibility between the compound represented by the formula (c3) and other components in the photosensitive composition, the number of carbon atoms in the alkyl group as R^{c4} is preferably 15 or less, and more preferably 10 or less.

When R^{c4} has a substituent, suitable examples of the substituent is a hydroxy group, an alkyl group having 1 or more and 20 or less carbon atoms, an alkoxy group having 1 or more and 20 or less carbon atoms, an aliphatic acyl group having 2 or more and 20 or less carbon atoms, an aliphatic acyloxy group having 2 or more and 20 or less carbon atoms, a phenoxy group, a benzoyl group, a benzoyloxy group, a group represented by — PO(OR)₂ (in which, R is an alkyl group having 1 or more and 6 or less carbon atoms), a halogen atom, a cyano group, a heterocyclyl group, and the like.

When R^{c4} is a heterocyclyl group, the heterocyclyl group may be an aliphatic heterocyclic group or an aromatic heterocyclic group. When R^{c4} is the heterocyclyl group, the heterocyclyl group is a 5- or 6-membered single ring containing one or more N, S, and O, or a heterocyclyl group in which single rings are fused each other, or a single ring is fused with a benzene ring. When the heterocyclyl group is a fused ring, the number of rings constituting the fused ring is 3 or less. Examples of the heterocycle constituting the heterocyclyl group include furan, thiophene, pyrrole, oxazole, isoxazole, thiazole, thiadiazole, isothiazole, imidazole, pyrazole, triazole, pyridine, pyrazine, pyrimidine, pyridazine, benzofuran, benzothiophene, indole, isoindole, indolizine, benzoimidazole, benzotriazole, benzoxazole, benzothiazole, carbazole, purine, quinoline, isoquinoline, quinazoline, phthalazine, cinnoline, quinoxaline, piperidine, piperazine, morpholine, piperidine, tetrahydropyran, tetrahydrofuran, and the like. When R^{c4} is the heterocyclyl group, examples of substituent that the heterocyclyl group may have include a hydroxy group, an alkoxy group having 1 or more and 6 or less carbon atoms, a halogen atom, a cyano group, a nitro group, and the like. Suitable examples of above described R^{c4} include methyl group, ethyl group, n-propyl group, isopropyl group, n-butyl group, isobutyl group, sec-butyl group, tert-butyl group, n-pentyl group, isopentyl group, neopentyl group, pentan-3-yl group, sec-pentyl group, tert-pentyl group, n-hexyl group, n-heptyl group, n-octyl group and 2-ethylhexyl group. Since solubility of the compound represented by the formula (c3) in the photosensitive composition is good, n-octyl group and 2-ethylhexyl group are preferred, and 2-ethylhexyl group is more preferred.

In the formula (c3), R^{c5} is a monovalent organic group. R^{c5} can be selected from various kinds of organic groups as long as it does not interfere with the object of the present invention. As the organic group, a carbon atom-containing group is preferred, and a group consisting of 1 or more carbon atoms, and 1 or more atoms selected from the group consisting of H, O, S, Se, N, B, P, Si and a halogen atom is more preferred. The number of carbon atoms in the carbon atom-containing group is not particularly limited, and is preferably 1 or more and 50 or less, and more preferably 1 or more and 20 or less. Examples of the organic group suitable for R^{c5} include an alkyl group, an alkoxy group, a cycloalkyl group, a cycloalkoxy group, a saturated aliphatic acyl group, an alkoxycarbonyl group, a saturated aliphatic acyloxy group, an optionally substituted phenyl group, an optionally substituted phenoxy group, an optionally substituted benzoyl group, an optionally substituted phenoxycarbonyl group, an optionally substituted benzoyloxy group, an optionally substituted phenylalkyl group, an optionally substituted naphthyl group, an optionally substituted naphthoxy group, an optionally substituted naphthoyl group, an optionally substituted naphthoxycarbonyl group, an optionally substituted naphthoyloxy group, an optionally substituted naphthylalkyl group, an optionally substituted heterocyclyl group, an optionally substituted heterocyclylcarbonyl group, an amino group substituted with 1 or 2 organic groups, morpholin-1-yl group, piperazin-1-yl group, a halogen atom, nitro group, cyano group, a substituent including a group represented by HX₂C— or H₂XC—(in which, X is each independently a halogen atom), and the like.

When R^{c5} is the alkyl group, a number of carbon atoms in the alkyl group is preferably 1 or more and 20 or less, and more preferably 1 or more and 6 or less. When R^{c5} is the alkyl group, the alkyl group may be linear or branched. Specific examples of the alkyl group as R^{c5} include methyl group, ethyl group, n-propyl group, isopropyl group, n-butyl group, isobutyl group, sec-butyl group, tert-butyl group, n-pentyl group, isopentyl group, sec-pentyl group, tert-pentyl group, n-hexyl group, n-heptyl group, n-octyl group, isooctyl group, sec-octyl group, tert-octyl group, n-nonyl group, isononyl group, n-decyl group, isodecyl group, and the like. When R^{c5} is alkyl group, the alkyl group may contain an ether bond (—O—) in the carbon chain. Examples of the alkyl group having an ether bond in a carbon chain include methoxyethyl group, ethoxyethyl group, methoxyethoxyethyl group, ethoxyethoxyethyl group, propyloxyethoxyethyl group, and methoxypropyl group.

When R^{c5} is the alkoxy group, the number of carbon atoms in the alkoxy group is preferably 1 or more and 20 or less, and more preferably 1 or more and 6 or less. When R^{c5} is the alkoxy group, the alkoxy group may be linear or branched. When R^{c5} is the alkoxy groups, specific examples thereof include methoxy group, ethoxy group, n-propyloxy group, isopropyloxy group, n-butyloxy group, isobutyloxy group, sec-butyloxy group, tert-butyloxy group, n-pentyloxy group, isopentyloxy group, sec-pentyloxy group, tert-pentyloxy group, n-hexyloxy group, n-heptyloxy group, n-octyloxy group, isooctyloxy group, sec-octyloxy group, tert-octyloxy group, n-nonyloxy group, isononyloxy group, n-decyloxy group, isodecyloxy group, and the like. When R^{c5} is the alkoxy groups, the alkoxy group may have an ether bond (—O—) in a carbon chain. Examples of the alkoxy group having an ether bond in a carbon chain include methoxyethoxy group, ethoxyethoxy group, methoxyethoxyethoxy group, ethoxyethoxyethoxy group, propyloxyethoxyethoxy group, and methoxypropyloxy group.

When R^{c5} is the cycloalkyl group or the cycloalkoxy group, a number of carbon atoms in the cycloalkyl group or the cycloalkoxy group is preferably 3 or more and 10 or less, and more preferably 3 or more and 6 or less. Specific examples of the cycloalkyl group as R^{c5} include cyclopropyl group, cyclobutyl group, cyclopentyl group, cyclohexyl group, cycloheptyl group, cyclooctyl group, and the like. Specific examples of the cycloalkoxy group as R^{c5} include cyclopropyloxy group, cyclobutyloxy group, cyclopentyloxy group, cyclohexyloxy group, cycloheptyloxy group, cyclooctyloxy group, and the like.

When R^{c5} is a saturated aliphatic acyl group or a saturated aliphatic acyloxy group, a number of carbon atoms is preferably 2 or more and 21 or less, and more preferably 2 or more and 7 or less. When R^{c5} is a saturated aliphatic acyl group, specific examples thereof include acetyl group, propanoyl group, n-butanoyl group, 2-methylpropanoyl group, n-pentanoyl group, 2,2-dimethylpropanoyl group, n-hexanoyl group, n-heptanoyl group, n-octanoyl group, n-nonanoyl group, n-decanoyl group, n-undecanoyl group, n-dodecanoyl group, n-tridecanoyl group, n-tetradecanoyl group, n-pentadecanoyl group, n-hexadecanoyl group, and the like. When R^{c5} is a saturated aliphatic acyloxy group, specific examples thereof include acetyloxy group, propanoyloxy group, n-butanoyloxy group, 2-methylpropanoyloxy group, n-pentanoyloxy group, 2,2-dimethylpropanoyloxy group, n-hexanoyloxy group, n-heptanoyloxy group, n-octanoyloxy group, n-nonanoyloxy group, n-decanoyloxy group, n-undecanoyloxy group, n-dodecanoyloxy group, n-tridecanoyloxy group, n-tetradecanoyloxy group, n-pentadecanoyloxy group, n-hexadecanoyloxy group, and the like.

When R^{c5} is an alkoxycarbonyl group, a number of carbon atoms is preferably 2 or more and 20 or less, and preferably 2 or more and 7 or less. When R^{c5} is an alkoxycarbonyl group, specific examples thereof include methoxycarbonyl group, ethoxycarbonyl group, n-propyloxycarbonyl group, isopropyloxycarbonyl group, n-butyloxycarbonyl group, isobutyloxycarbonyl group, sec-butyloxycarbonyl group, tert-butyloxycarbonyl group, n-pentyloxycarbonyl group, isopentyloxycarbonyl group, sec-pentyloxycarbonyl group, tert-pentyloxycarbonyl group, n-hexyloxycarbonyl group, n-heptyloxycarbonyl group, n-octyloxycarbonyl group, isooctyloxycarbonyl group, sec-octyloxycarbonyl group, tert-octyloxycarbonyl group, n-nonyloxycarbonyl group, isononyloxycarbonyl group, n-decyloxycarbonyl group, and isodecyloxycarbonyl group.

When R^{c5} is a phenylalkyl group, a number of carbon atoms in the phenylalkyl group is preferably 7 or more and 20 or less, and more preferably 7 or more and 10 or less. In addition, when R^{c5} is a naphthylalkyl group, a number of carbon atoms in the naphthylalkyl group is preferably 11 or more and 20 or less, and more preferably 11 or more and 14 or less. When R^{c5} is a phenylalkyl group, specific examples thereof include benzyl group, 2-phenylethyl group, 3-phenylpropyl group, and 4-phenylbutyl group. When R^{c5} is a naphthylalkyl group, specific examples thereof include α-naphthylmethyl group, β-naphthylmethyl group, 2-(α-naphthyl)ethyl group, and 2-(β-naphthyl)ethyl group. When R^{c5} is a phenylalkyl group or a naphthylalkyl group, R^{c5} may further have a substituent on phenyl group or naphthyl group.

When R^{c5} is a heterocyclyl group, the heterocyclyl group is the same as the heterocyclyl group as R^{c4} in the formula (c3), and may further have a substituent. When R^{c5} is a heterocyclylcarbonyl group, the heterocyclyl group included in the heterocyclylcarbonyl group is the same as the heterocyclyl group as R^{c5}.

When R^{c5} is an amino group substituted with one or two organic groups, suitable examples of the organic groups include an alkyl group having 1 or more and 20 or less carbon atoms, a cycloalkyl group having 3 or more and 10 or less carbon atoms, a saturated aliphatic acyl group having 2 or more and 21 or less carbon atoms, an optionally substituted phenyl group, an optionally substituted benzoyl group, an optionally substituted phenylalkyl group having 7 or more and 20 or less carbon atoms, an optionally substituted naphthyl group, an optionally substituted naphthoyl group, an optionally substituted naphthylalkyl group having 11 or more and 20 or less carbon atoms, and a heterocyclyl group. Specific examples of these suitable organic group are the same as R^{c5}. Specific examples of the amino group substituted with one or two organic groups include methylamino group, ethylamino group, diethylamino group, n-propylamino group, di-n-propylamino group, isopropylamino group, n-butylamino group, di-n-butylamino group, n-pentylamino group, n-hexylamino group, n-heptylamino group, n-octylamino group, n-nonylamino group, n-decylamino group, phenylamino group, naphthylamino group, acetylamino group, propanoylamino group, n-butanoylamino group, n-pentanoylamino group, n-hexanoylamino group, n-heptanoylamino group, n-octanoylamino group, n-decanoylamino group, benzoylamino group, α-naphthoylamino group, β-naphthoylamino group, and the like.

When the phenyl group, the naphthyl group, and the heterocyclyl group included in R^{c5} further have a substituent, examples of the substituent include a substituent including a group represented by HX₂C— or H₂XC—(for example, a halogenated alkoxy group including a group represented by HX₂C— or H₂XC—, and a halogenated alkyl group including a group represented by HX₂C— or H₂XC—), an alkyl group having 1 or more and 6 or less carbon atoms, an alkoxy group having 1 or more and 6 or less carbon atoms, a saturated aliphatic acyl group having 2 or more and 7 or less carbon atoms, an alkoxycarbonyl group having 2 or more and 7 or less carbon atoms, a saturated aliphatic acyloxy group having 2 or more and 7 or less carbon atoms, a monoalkylamino group having an alkyl group having 1 or more and 6 or less carbon atoms, a dialkylamino group having alkyl groups having 1 or more and 6 or less carbon atoms, morpholin-1-yl group, piperazin-1-yl group, benzoyl group, a halogen atom, nitro group, cyano group, and the like. When the phenyl group, the naphthyl group, and the heterocyclyl group included in R^{c5} further having one or more substituents, the number of substituents is not particularly limited as long as it does not interfere with the object of the present invention, and is preferably 1 or more and 4 or less. When a phenyl group, a naphthyl group, and a heterocyclyl group included in R^{c5} have a plurality of substituents, the plurality of substituents may be the same or different.

When a benzoyl group, a naphthyl group included in R^{c5} further have a substituent, examples of the substituent include an alkyl group having 1 or more and 6 or less carbon atoms, a morpholin-1-yl group, a piperazin-1-yl group, a 2-thenoyl group (thiophen-2-ylcarbonyl group), a furan-3-ylcarbonyl group, a phenyl group, and the like.

As a halogen atom represented by X, a fluorine atom, a chlorine atom, a bromine atom, and the like are exemplified, and the fluorine atom is preferred.

As a substituent including a group represented by HX₂C— or H₂XC—, a halogenated alkoxy group including a group represented by HX₂C— or H₂XC—, a group having a halogenated alkoxy group including a group represented by HX₂C— or H₂XC—, a halogenated alkyl group including a group represented by HX₂C— or H₂XC—, a group having a halogenated alkyl group including a group represented by HX₂C— or H₂XC—, and the like are exemplified, and the halogenated alkoxy group including a group represented by HX₂C— or H₂XC— or the group having a halogenated alkoxy group including a group represented by HX₂C— or H₂XC— is preferred.

As a group having a halogenated alkyl group including a group represented by HX₂C— or H₂XC—, an aromatic group, such as phenyl group, and naphthyl group, substituted with the halogenated alkyl group including a group represented by HX₂C— or H₂XC—, a cycloalkyl group, such as cyclopentyl group, and cyclohexyl group, substituted with the halogenated alkyl group including a group represented by HX₂C— or H₂XC—, and the like are exemplified, and the aromatic group substituted with the halogenated alkyl group including a group represented by HX₂C— or H₂XC— is preferred.

As a group having a halogenated alkoxy group including a group represented by HX₂C— or H₂XC—, an aromatic group, such as phenyl group, and naphthyl group, substituted with the halogenated alkoxy group including a group represented by HX₂C— or H₂XC—, an alkyl group, such as methyl group, ethyl group, n-propyl group, and i-propyl group, substituted with the halogenated alkoxy group including a group represented by HX₂C— or H₂XC—, a cycloalkyl group, such as cyclopentyl group, and cyclohexyl group, substituted with the halogenated alkoxy group including a group represented by HX₂C— or H₂XC—, and the like are exemplified, and the aromatic group substituted with the halogenated alkoxy group including a group represented by HX₂C— or H₂XC—.

In addition, a cycloalkyl group, a phenoxyalkyl group that may have a substituent on an aromatic ring, and a phenylthioalkyl group that may have a substituent on an aromatic ring are also preferred as R^{c5}. The substituent that the phenoxyalkyl group and phenylthioalkyl group may have is the same as the substituent that the phenyl group included in R^{c5} may have.

Among the monovalent organic groups, an alkyl group, a cycloalkyl group, an optionally substituted phenyl group or cycloalkyl group, and phenylthioalkyl group that may have a substituent on an aromatic ring are preferred. As the alkyl group, an alkyl group having 1 or more and 20 or less carbon atoms is preferred, an alkyl group having 1 or more and 8 or less carbon atoms is more preferred, an alkyl group having 1 or more and 4 or less carbon atoms is particularly preferred, and methyl group is most preferred. Among the optionally substituted phenyl groups, methylphenyl group is preferred, and 2-methylphenyl group is more preferred. The number of carbon atoms in the cycloalkyl group included in the cycloalkylalkyl group is preferably 5 or more and 10 or less, more preferably 5 or more and 8 or less, and particularly preferably 5 or 6. The number of carbon atoms in the alkylene group included in the cycloalkylalkyl group is preferably 1 or more and 8 or less, more preferably 1 or more and 4 or less, and particularly preferably 2. Among the cycloalkylalkyl groups, cyclopentylethyl group is preferred. The number of carbon atoms in the alkylene group included in the phenylthioalkyl group that may have a substituent on the aromatic ring is preferably 1 or more and 8 or less, more preferably 1 or more and 4 or less, and particularly preferably 2. Among the phenylthioalkyl groups that may have a substituent on the aromatic ring, 2-(4-chlorophenylthio)ethyl group is preferred.

In the group represented by the formula (c3), when there is a plurality of R^{c5}s and the plurality of R^{c5}s bonds to each other to form a ring, examples of the ring formed include a hydrocarbon ring, a heterocyclic ring, and the like. As a heteroatom included in the heterocycle, for example, N, O, and S is exemplified. An aromatic ring is particularly preferred as the ring formed by combining a plurality of R^{c5}s each other. Such an aromatic ring may be an aromatic hydrocarbon ring or an aromatic heterocyclic ring. Such an aromatic ring is preferably an aromatic hydrocarbon ring. Specific examples of the group in which a benzene ring is formed by combining a plurality of R^{c5}s in the formula (3) each other are shown below.

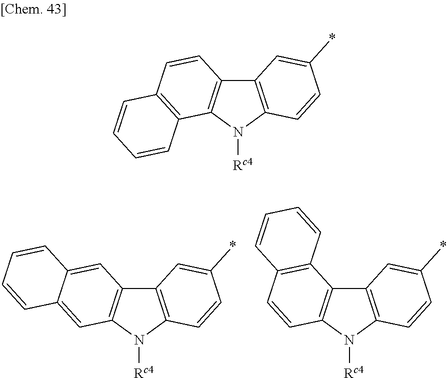

In a group represented by the formula (c4), R^{c8} is a nitro group or a monovalent organic group. R^{c8} bonds to a 6-membered aromatic ring other than an aromatic ring that bonds to a group represented by —(CO)ₙ₁— on a condensed ring in the formula (c4). In the formula (c4), the bond position of R^{c8} is not particularly limited. When a group represented by the formula (c4) has 1 or more R^{c8}s, one of 1 or more R^{c8}s preferably bonds to 7-position in a fluorene skeleton from the viewpoint that a compound having the group represented by the formula (c4) can be easily synthesized. In other words, when a group represented by the formula (c4) has 1 or more R^{c8}s, the group represented by the formula (c4) is preferably a group represented by following formula (c6). When there is a plurality of R^{c8}s, plurality of substituents may be the same or different.

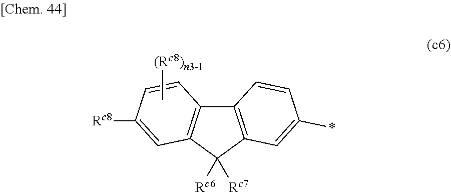

In the formula (c6), R^{c6}, R^{c7}, R^{c8}, and n3 are same as R^{c6}, R^{c7}, R^{c8}, and n3 in the formula (c4).

When R^{c8} is the monovalent organic group, R^{c8} is not particularly limited as long as it does not interfere with the object of the present invention. As the organic group, a carbon atom-containing group is preferred, and a group consisting of 1 or more carbon atoms, and 1 or more atoms selected from the group consisting of H, O, S, Se, N, B, P, Si and a halogen atom is more preferred. The number of carbon atoms in the carbon atom-containing group is not particularly limited, and is preferably 1 or more and 50 or less, and more preferably 1 or more and 20 or less. Suitable examples of the monovalent organic group as R^{c8} include the same groups as the examples of the monovalent organic group as R^{c5} in the formula (c3).

In the formula (c4), R^{c6} and R^{c7} each represent an optionally substituted chain alkyl group, an optionally substituted chain alkoxy group, an optionally substituted cyclic organic group, or hydrogen atom. R^{c6} and R^{c7} may be combined to one another to form a ring. Among these groups, R^{c6} and R^{c7} are preferably the optionally substituted chain alkyl groups. When R^{c6} and R^{c7} are the optionally substituted chain alkyl groups, the chain alkyl group may be either a straight-chain alkyl group or a branched-chain alkyl group.

When R^{c6} and R^{c7} are chain alkyl groups having no substituent, the number of carbon atoms in the chain alkyl group is preferably 1 or more and 20 or less, more preferably 1 or more and 10 or less, and particularly preferably 1 or more and 6 or less. When R^{c6} and R^{c7} are chain alkyl groups, specific examples thereof include methyl group, ethyl group, n-propyl group, isopropyl group, n-butyl group, isobutyl group, sec-butyl group, tert-butyl group, n-pentyl group, isopentyl group, sec-pentyl group, tert-pentyl group, n-hexyl group, n-heptyl group, n-octyl group, isooctyl group, sec-octyl group, tert-octyl group, n-nonyl group, isononyl group, n-decyl group, isodecyl group, and the like. When R^{c6} and R^{c7} are the alkyl group, the alkyl group may have an ether bond (—O—) in a carbon chain. Examples of the alkyl group having an ether bond in a carbon chain include methoxyethyl group, ethoxyethyl group, methoxyethoxyethyl group, ethoxyethoxyethyl group, propyloxyethoxyethyl group, and methoxypropyl group.

When R^{c6} and R^{c7} are chain alkyl group having a substituent, the number of carbon atoms in the chain alkyl group is preferably 1 or more and 20 or less, more preferably 1 or more and 10 or less, and particularly preferably 1 or more and 6 or less. In this case, the number of carbon atoms in the substituent is not included in the number of carbon atoms in the chain alkyl group. The chain alkyl group having a substituent is preferably a straight-chain group.

The substituent, with which the alkyl group is optionally substituted, is not particularly limited as long as it does not interfere with the object of the present invention. Suitable examples of the substituent include an alkoxy group, cyano group, a halogen atom, a halogenated alkyl group, a cyclic organic group, and an alkoxycarbonyl group. Examples of the halogen atom include fluorine atom, chlorine atom, bromine atom, and iodine atom. Among these, fluorine atom, chlorine atom, and bromine atom are preferred. Examples of the cyclic organic group include a cycloalkyl group, an aromatic hydrocarbon group, and a heterocyclyl group. Specific examples of the cycloalkyl group are the same as suitable examples in case R^{c8} is a cycloalkyl group. Specific examples of the aromatic hydrocarbon group include phenyl group, naphthyl group, biphenylyl group, anthryl group, phenanthryl group, and the like. Specific examples of the heterocyclyl group are the same as suitable examples in case RCS is a heterocyclyl group. When RCS is an alkoxycarbonyl group, an alkoxy group included in the alkoxycarbonyl group may be straight or branched, and preferably straight. The number of carbon atoms in an alkoxy group included in the alkoxycarbonyl group is preferably 1 or more and 10 or less, and more preferably 1 or more and 6 or less.

When the chain alkyl group has a substituent, the number of substituents is not particularly limited. The number of substituents preferably varies depending on the number of carbon atoms in the chain alkyl group. The number of substituents is typically 1 or more and 20 or less, preferably 1 or more and 10 or less, and more preferably 1 or more and 6 or less.

When R^{c6} and R^{c7} are unsubstituted chain alkoxy group, the number of carbon atoms in the chain alkoxy group is preferably 1 or more and 20 or less, more preferably 1 or more and 10 or less, and particularly preferably 1 or more and 6 or less. When R^{c6} and R^{c7} are chain alkoxy group, specific examples thereof include methoxy group, ethoxy group, n-propyloxy group, isopropyloxy group, n-butyloxy group, isobutyloxy group, sec-butyloxy group, tert-butyloxy group, n-pentyloxy group, isopentyloxy group, sec-pentyloxy group, tert-pentyloxy group, n-hexyloxy group, n-heptyloxy group, n-octyloxy group, isooctyloxy group, sec-octyloxy group, tert-octyloxy group, n-nonyloxy group, isononyloxy group, n-decyloxy group, isodecyloxy group, and the like. In addition, when R^{c6} and R^{c7} are alkoxy group, the alkoxy group may include an ether bond (—O—) in the carbon chain. Examples of the alkoxy group having an ether bond in a carbon chain include methoxyethoxy group, ethoxyethoxy group, methoxyethoxyethoxy group, ethoxyethoxyethoxy group, propyloxyethoxyethoxy group, and methoxypropyloxy group.

When R^{c6} and R^{c7} are chain alkoxy groups having a substituent, the substituent that the alkoxy group may have is the same as the substituent that the chain alkyl group as R^{c6} and R^{c7} may have.

When R^{c6} and R^{c7} are cyclic organic group, the cyclic organic group may be an alicyclic group or an aromatic group. Examples of the cyclic organic group include an aliphatic cyclic hydrocarbon group, an aromatic hydrocarbon group, and a heterocyclyl group. When R^{c6} and R^{c7} are cyclic organic group, the substituent, with which the cyclic organic group is optionally substituted, is the same as in the case where R^{c6} and R^{c7} are chain alkyl groups.

When R^{c6} and R^{c7} are aromatic hydrocarbon groups, the aromatic hydrocarbon group is preferably a phenyl group, or a group formed by bonding a plurality of benzene rings through a carbon-carbon bond, or a group formed by condensing a plurality of benzene rings. When the aromatic hydrocarbon group is a phenyl group, or a group formed by bonding or condensing a plurality of benzene rings, the number of benzene rings included in the aromatic hydrocarbon group is not particularly limited, and is preferably 3 or less, more preferably 2 or less, and particularly preferably 1. Suitable specific examples of the aromatic hydrocarbon group include phenyl group, naphthyl group, biphenylyl group, anthryl group, and phenanthryl group.

When R^{c6} and R^{c7} are aliphatic cyclic hydrocarbon groups, the aliphatic cyclic hydrocarbon group may be a monocyclic or polycyclic group. The number of carbon atoms in the aliphatic cyclic hydrocarbon group is not particularly limited, and is preferably 3 or more and 20 or less, and more preferably 3 or more and 10 or less. Examples of the monocyclic cyclic hydrocarbon group include cyclopropyl group, cyclobutyl group, cyclopentyl group, cyclohexyl group, cycloheptyl group, cyclooctyl group, norbornyl group, isobornyl group, tricyclononyl group, tricyclodecyl group, tetracyclododecyl group, and adamantyl group.

When R^{c6} and R^{c7} are heterocyclyl groups, the same groups as the heterocyclyl groups as R^{c5} in the formula (c3) are exemplified.

R^{c6} and R^{c7} may be combined to one another to form a ring. The group composed of the ring formed by R^{c6} and R^{c7} is preferably a cycloalkylidene group. When R^{c6} and R^{c7} are combined to form a cycloalkylidene group, the ring constituting the cycloalkylidene group is preferably a 5- to 6-membered ring, and more preferably a 5-membered ring.

When R^{c7} and a benzene ring in fluorene skeleton are combined to form a ring, the ring may be an aromatic ring or an aliphatic ring.

When the group formed by combining R^{c6} and R^{c7} is a cycloalkylidene group, the cycloalkylidene group may be condensed with one or more other rings. Examples of the ring which may be condensed with the cycloalkylidene group include benzene ring, naphthalene ring, cyclobutane ring, cyclopentane ring, cyclohexane ring, cycloheptane ring, cyclooctane ring, furan ring, thiophene ring, pyrrole ring, pyridine ring, pyrazine ring, pyrimidine ring, and the like.

Examples of a suitable group among R^{c6} and R^{c7} described above include a group represented by the formula: -A¹-A². In the formula, A¹ is a linear alkylene group. A² is an alkoxy group, a cyano group, a halogen atom, a halogenated alkyl group, a cyclic organic group, or an alkoxycarbonyl group.

The number of carbon atoms in the linear alkylene group for A¹ is preferably 1 or more and 10 or less, and more preferably 1 or more and 6 or less. When A² is an alkoxy group, the alkoxy group may be a linear or branched alkoxy group, and preferably a linear alkoxy group. The number of carbon atoms in the alkoxy group is preferably 1 or more and 10 or less, and more preferably 1 or more and 6 or less. When A² is a halogen atom, fluorine atom, chlorine atom, bromine atom, or iodine atom is preferred, and fluorine atom, chlorine atom, or bromine atom is more preferred. When A² is a halogenated alkyl group, a halogen atom included in the halogenated alkyl group is preferably fluorine atom, chlorine atom, bromine atom, or iodine atom, and more preferably is fluorine atom, chlorine atom, or bromine atom. The halogenated alkyl group may be a linear or branched halogenated alkyl group, preferably a linear halogenated alkyl group. When A² is a cyclic organic group, examples of the cyclic organic group are the same as the cyclic organic group possessed by R^{c6} and R^{c7} as a substituent. When A² is an alkoxycarbonyl group, examples of the alkoxycarbonyl group are the same as the alkoxycarbonyl group possessed by R^{c6} and R^{c7} as a substituent.

Suitable specific examples of R^{c6} and R^{c7} include alkyl groups such as ethyl group, n-propyl group, n-butyl group, n-hexyl group, n-heptyl group, and n-octyl group; alkoxyalkyl groups such as 2-methoxyethyl group, 3-methoxy-n-propyl group, 4-methoxy-n-butyl group, 5-methoxy-n-pentyl group, 6-methoxy-n-hexyl group, 7-methoxy-n-heptyl group, 8-methoxy-n-octyl group, 2-ethoxyethyl group, 3-ethoxy-n-propyl group, 4-ethoxy-n-butyl group, 5-ethoxy-n-pentyl group, 6-ethoxy-n-hexyl group, 7-ethoxy-n-heptyl group, and 8-ethoxy-n-octyl group; cyanoalkyl groups such as 2-cyanoethyl group, 3-cyano-n-propyl group, 4-cyano-n-butyl group, 5-cyano-n-pentyl group, 6-cyano-n-hexyl group, 7-cyano-n-heptyl group, and 8-cyano-n-octyl group; phenylalkyl groups such as 2-phenylethyl group, 3-phenyl-n-propyl group, 4-phenyl-n-butyl group, 5-phenyl-n-pentyl group, 6-phenyl-n-hexyl group, 7-phenyl-n-heptyl group, and 8-phenyl-n-octyl group; cycloalkylalkyl groups such as 2-cyclohexylethyl group, 3-cyclohexyl-n-propyl group, 4-cyclohexyl-n-butyl group, 5-cyclohexyl-n-pentyl group, 6-cyclohexyl-n-hexyl group, 7-cyclohexyl-n-heptyl group, 8-cyclohexyl-n-octyl group, 2-cyclopentylethyl group, 3-cyclopentyl-n-propyl group, 4-cyclopentyl-n-butyl group, 5-cyclopentyl-n-pentyl group, 6-cyclopentyl-n-hexyl group, 7-cyclopentyl-n-heptyl group, and 8-cyclopentyl-n-octyl group; alkoxycarbonylalkyl groups such as 2-methoxycarbonylethyl group, 3-methoxycarbonyl-n-propyl group, 4-methoxycarbonyl-n-butyl group, 5-methoxycarbonyl-n-pentyl group, 6-methoxycarbonyl-n-hexyl group, 7-methoxycarbonyl-n-heptyl group, 8-methoxycarbonyl-n-octyl group, 2-ethoxycarbonylethyl group, 3-ethoxycarbonyl-n-propyl group, 4-ethoxycarbonyl-n-butyl group, 5-ethoxycarbonyl-n-pentyl group, 6-ethoxycarbonyl-n-hexyl group, 7-ethoxycarbonyl-n-heptyl group, and 8-ethoxycarbonyl-n-octyl group; and halogenated alkyl groups such as 2-chloroethyl group, 3-chloro-n-propyl group, 4-chloro-n-butyl group, 5-chloro-n-pentyl group, 6-chloro-n-hexyl group, a 7-chloro-n-heptyl group, 8-chloro-n-octyl group, 2-bromoethyl group, 3-bromo-n-propyl group, 4-bromo-n-butyl group, 5-bromo-n-pentyl group, 6-bromo-n-hexyl group, 7-bromo-n-heptyl group, 8-bromo-n-octyl group, 3,3,3-trifluoropropyl group, and 3,3,4,4,5,5,5-heptafluoro-n-pentyl group.

Among groups mentioned above, Suitable groups as R^{c6} and R^{c7} are ethyl group, n-propyl group, n-butyl group, n-pentyl group, 2-methoxyethyl group, 2-cyanoethyl group, 2-phenylethyl group, 2-cyclohexylethyl group, 2-methoxycarbonylethyl group, 2-chloroethyl group, 2-bromoethyl group, 3,3,3-trifluoropropyl group, and 3,3,4,4,5,5,5-heptafluoro-n-pentyl group.

From the view point that a highly sensitive photo polymerization initiator is likely to be easily obtained, A is preferably S.

In the formula (c5), R^{c9} is a monovalent organic group, a halogen atom, nitro group, or cyano group. When R^{c9} in the formula (c5) is the monovalent organic group, the monovalent organic group can be selected from various organic groups as long as it does not interfere with the object of the present invention. As the organic group, a carbon atom-containing group is preferred, and a group consisting of one or more carbon atoms, and one or more atoms selected from the group consisting of H, O, S, Se, N, B, P, Si and a halogen atom is more preferred. The number of carbon atoms in the carbon atom-containing group is not particularly limited, and preferably 1 or more and 50 or less, and more preferably 1 or more and 20 or less. Suitable examples of the organic group as R^{c9} in the formula (c5) are the same groups as the monovalent organic groups as R^{c5} in the formula (c5)

Among R^{c9}, benzoyl group; naphthoyl group; benzoyl groups substituted with a group selected from the group consisting of an alkyl group having 1 or more and 6 or less carbon atoms, morpholin-1-yl group, piperazin-1-yl group, and a phenyl group; nitro group; optionally substituted benzofuranylcarbonyl group are preferred, and benzoyl group; naphthoyl group; 2-methylphenylcarbonyl group; 4-(piperazin-1-yl)phenylcarbonyl group; and 4-(phenyl)phenylcarbonyl group are more preferred.

In addition, in the formula (c5), n4 is preferably an integer of 0 or more and 3 or less, more preferably an integer of 0 or more and 2 or less, and particularly preferably 0 or 1. When n4 is 1, the position at which R^{c9} bonds is preferably the para-position relative to the bonding through which the phenyl group (to which R^{c9} bonds) bonds to an oxygen atom or a sulfur atom.

In the formula (c1) and the formula (c2), the monovalent organic group as R^{c2} is not particularly limited as long as it does not interfere with the object of the present invention. As the organic group, a carbon atom-containing group is preferred, and a group consisting of one or more carbon atoms, and one or more atoms selected from the group consisting of H, O, S, Se, N, B, P, Si and a halogen atom is more preferred. The number of carbon atoms in the carbon atom-containing group is not particularly limited, and preferably 1 or more and 50 or less, and more preferably 1 or more and 20 or less. Suitable examples of the monovalent organic group as R^{c2} are the same groups as the monovalent organic groups as R^{c5} in the formula (c3). Specific examples of these groups are the same as the groups described for R^{c5} in the formula (c3). In addition, a cycloalkyl group, a phenoxyalkyl group that may have a substituent on an aromatic ring, and a phenylthioalkyl group that may have a substituent on an aromatic ring are also preferred as R^{c2}. The substituent that the phenoxyalkyl group and phenylthioalkyl group may have is the same as the substituent that the phenyl group, the naphthyl group, and the heterocyclyl group included in R^{c5} in the formula (c3) may have.

Among substituents, a substituent including the group represented by HX₂C— or H₂XC— described above, an alkyl group, a cycloalkyl group, an optionally substituted phenyl group, an optionally substituted cycloalkyl group, and a phenylthioalkyl group that may have a substituent on an aromatic ring are preferred. The alkyl group, the optionally substituted alkyl group, the number of carbon atoms in the cycloalkyl group included in the cycloalkylalkyl group, the cycloalkylalkyl group, the number of carbon atoms in the alkylene group included in the phenylthioalkyl group that may have a substituent on an aromatic ring, and the phenylthioalkyl group that may have a substituent on an aromatic ring are the same as these about R^{c5}.

A group represented by -A³-CO—O-A⁴ is also preferred as R^{c2}. A³ is a divalent organic group, preferably a divalent hydrocarbon group, and more preferably an alkylene group. A⁴ is a monovalent organic group, and preferably a monovalent hydrocarbon group.

When A³ is the alkylene group, the alkylene group may be straight or branched, and is preferably straight. When A³ is the alkylene group, the number of carbon atoms in the alkylene group is preferably 1 or more and 10 or less, more preferably 1 or more and 6 or less, and particularly preferably 1 or more and 4 or less.

Suitable examples of A⁴ include an alkyl group having 1 or more and 10 or less carbon atoms, an aralkyl group having 7 or more and 20 or less carbon atoms, and an aromatic hydrocarbon group having 6 or more and 20 or less carbon atoms. Suitable specific examples of A⁴ include methyl group, ethyl group, n-propyl group, isopropyl group, n-butyl group, isobutyl group, sec-butyl group, tert-butyl group, n-pentyl group, n-hexyl group, phenyl group, naphthyl group, benzyl group, phenethyl group, α-naphthylmethyl group, β-naphthylmethyl group, and the like.

Specific examples of a suitable group represented by -A³-CO—O-A⁴ include 2-methoxycarbonylethyl group, 2-ethoxycarbonylethyl group, 2-n-propyloxycarbonylethyl group, 2-n-butyloxycarbonylethyl group, 2-n-pentyloxycarbonylethyl group, 2-n-hexyloxycarbonylethyl group, 2-benzyloxycarbonylethyl group, 2-phenoxycarbonylethyl group, 3-methoxycarbonyl-n-propyl group, 3-ethoxycarbonyl-n-propyl group, n-propyloxycarbonyl-n-propyl group, 3-n-butyloxycarbonyl-n-propyl group, 3-n-pentyloxycarbonyl-n-propyl group, 3-n-hexyloxycarbonyl-n-propyl group, 3-benzyloxycarbonyl-n-propyl group, 3-phenoxycarbonyl-n-propyl group, and the like.

A group represented by the following formula (c7) or the following formula (c8) is also preferred as R^{c2}.

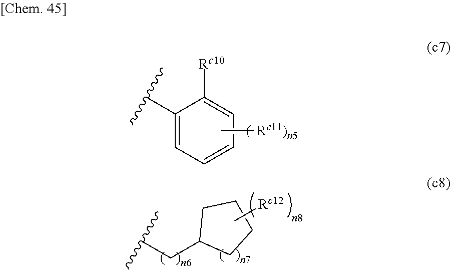

In the formulas (c7) and (c8), R^{c10} and R^{c11} are each independently a monovalent organic group. n5 is an integer of 0 or more and 4 or less. when R^{c10} and R^{c11} are adjacent to each other on the benzene ring, R^{c10} and R^{c11} may be combined to each other to form a ring. R^{c12} is a monovalent organic group. n6 is an integer of 1 or more and 8 or less. n7 is an integer of 1 or more and 5 or less. n8 is an integer of 0 or more and (n7+3) or less.

The organic group as R^{c10} and R^{c11} in the formula (c7) is the same as R^{c8} in the formula (c4). As R^{c10}, a halogenated alkoxy group including a group represented by HX₂C— or H₂XC—, a halogenated alkyl group including a group represented by HX₂C— or H₂XC—, an alkyl group, or a phenyl group is preferred. When R^{c10} and R^{c11} are combined to each other to form a ring, the ring may be an aromatic ring or an aliphatic ring. Suitable examples of the group represented by the formula (c7) in which R^{c10} and R^{c11} form a ring include naphthalen-1-yl group, 1,2,3,4-tetrahydronaphthalen-5-yl group, and the like. In the formula (c7), n5 is an integer of 0 or more and 5 or less, preferably 0 or 1, and more preferably 0.

In the above formula (c8), R^{c12} is an organic group. As the organic group, the same group as the organic group described above as R^{c8} in the formula (c4) is exemplified. Among the organic groups, an alkyl group is preferred. The alkyl group may be straight or branched. The number of carbon atoms in the alkyl group is preferably 1 or more and 10 or less, more preferably 1 or more and 5 or less, and particularly preferably 1 or more and 3 or less. Methyl group, ethyl group, propyl group, isopropyl group, butyl group, and the like are preferably exemplified as R^{c12}. Among these, methyl group is preferred.

In the above formula (c8), n7 is an integer of 1 or more and 5 or less, preferably an integer of 1 or more and 3 or less, and more preferably 1 or 2. In the above formula (c8), n8 is 0 or more and (n7+3) or less, preferably an integer of 0 or more and 3 or less, more preferably an integer of 0 or more and 2 or less, and particularly preferably 0. In the above formula (c8), n6 is an integer of 1 or more and 8 or less, preferably an integer of 1 or more and 5 or less, further preferably an integer of 1 or more and 3 or less, and particularly preferably 1 or 2.

In the formula (c2), R^{c3} is hydrogen atom, an optionally substituted aliphatic hydrocarbon group having 1 or more and 20 or less carbon atoms, or an optionally substituted aryl group. When R^{c3} is the aliphatic hydrocarbon group, preferable examples of the substituent which may be possessed by the aliphatic hydrocarbon group includes phenyl group, naphthyl group and the like.

In the formula (c1) and (c2), suitable examples of R^{c3} include hydrogen atom, methyl group, ethyl group, n-propyl group, isopropyl group, n-butyl group, 2-cyclopentylethyl group, 2-cyclobutylethyl group, cyclohexylmethyl group, phenyl group, benzyl group, methylphenyl group, naphthyl group, and the like. Among these, methyl group or phenyl group is more preferred.

Preferable specific examples of the compound represented by the formula (c2) and having the group represented by the formula (c3) as R^{c1} include the following compounds.

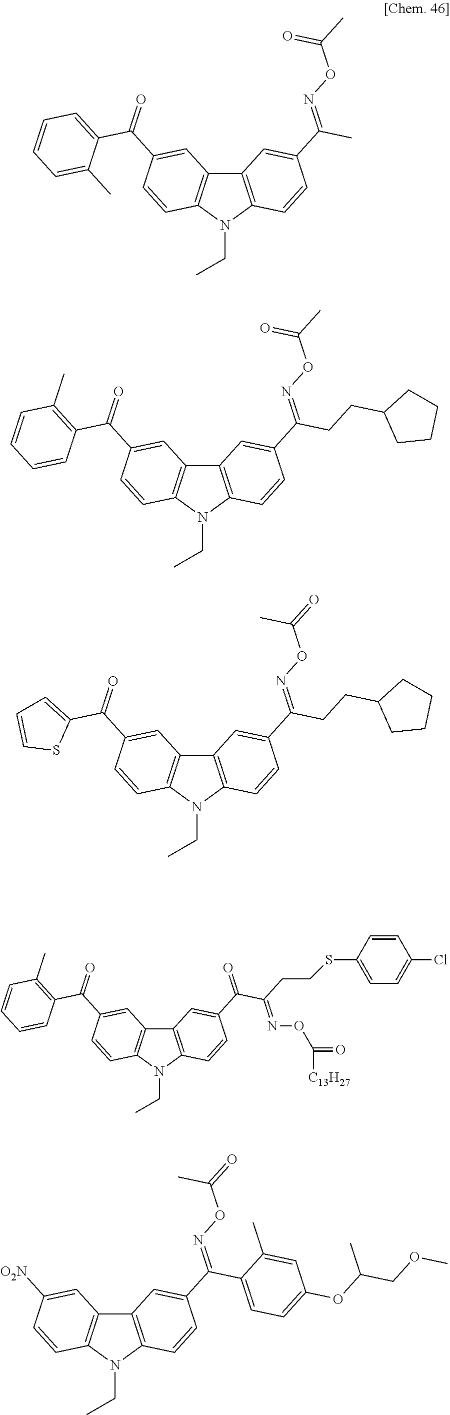

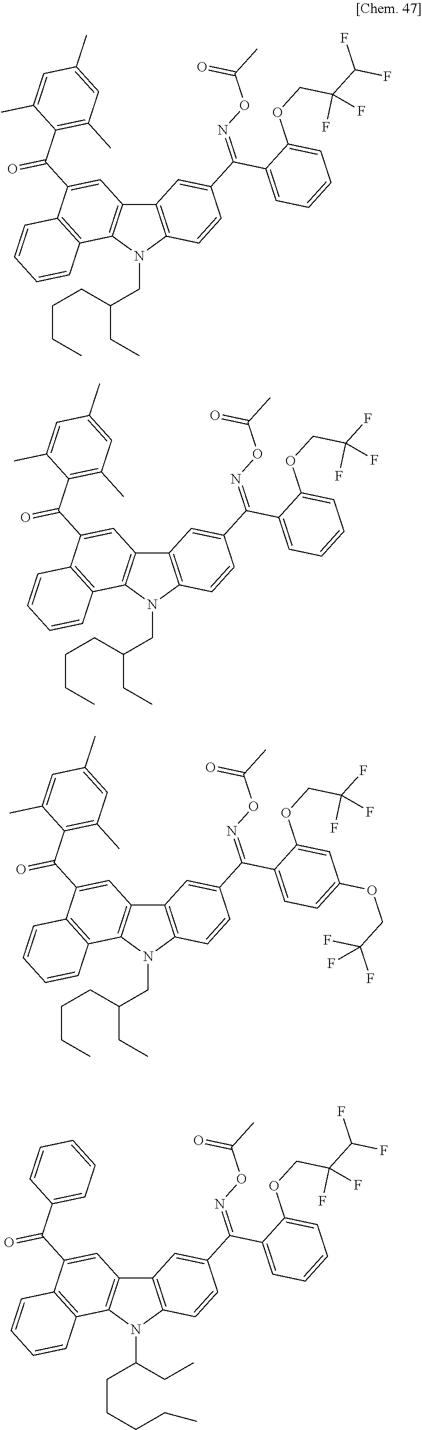

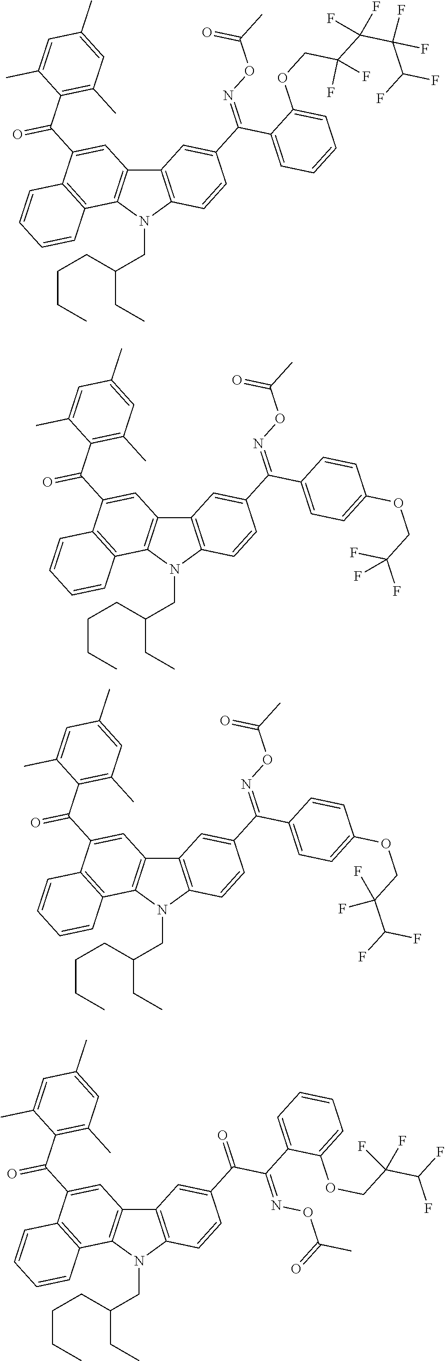

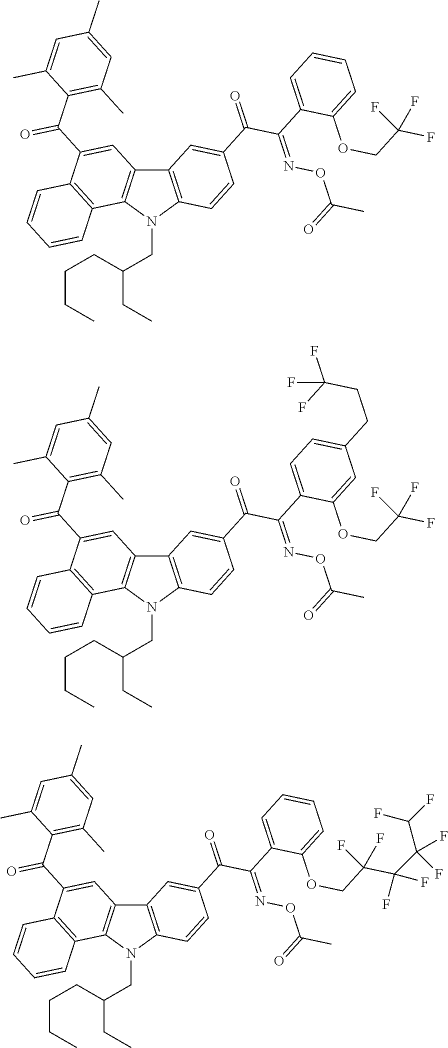

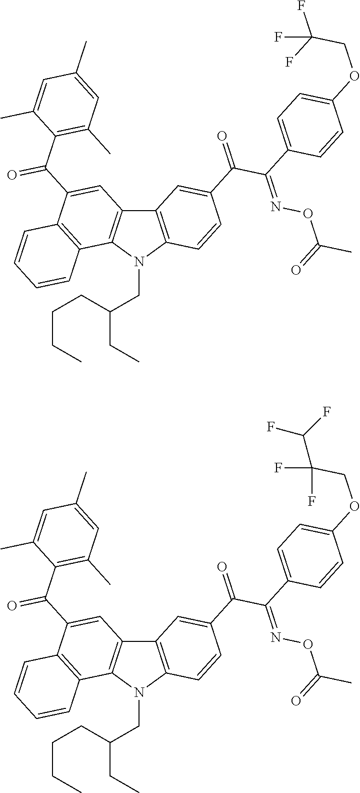 [SMILES: CCCCC(CC)Cn1c2ccc(C(=O)/C(=N/OC(C)=O)c3ccc(OCC(F)(F)F)cc3)cc2c2cc(C(=O)c3c(C)cc(C)cc3C)c3ccccc3c21 ; CCCCC(CC)Cn1c2ccc(C(=O)/C(=N/OC(C)=O)c3ccc(OCC(F)(F)C(F)F)cc3)cc2c2cc(C(=O)c3c(C)cc(C)cc3C)c3ccccc3c21]

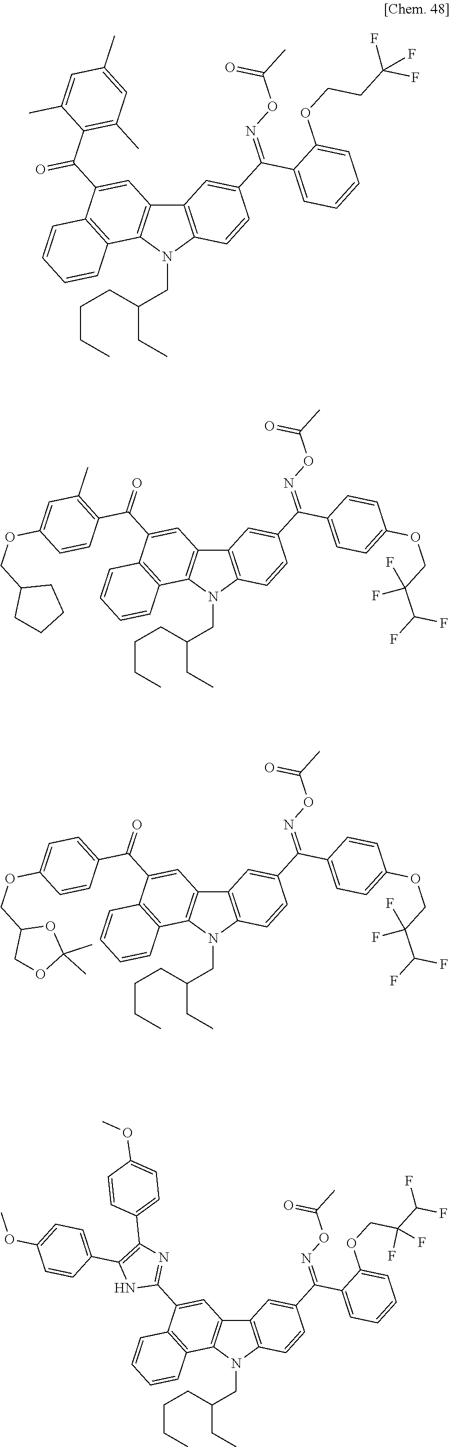 [SMILES: CCCCC(CC)Cn1c2ccc(/C(=N/OC(C)=O)c3ccccc3OCCC(F)(F)F)cc2c2cc(C(=O)c3c(C)cc(C)cc3C)c3ccccc3c21 ; CCCCC(CC)Cn1c2ccc(/C(=N/OC(C)=O)c3ccc(OCC(F)(F)C(F)F)cc3)cc2c2cc(C(=O)c3ccc(OCC4CCCC4)cc3C)c3ccccc3c21 ; CCCCC(CC)Cn1c2ccc(/C(=N/OC(C)=O)c3ccc(OCC(F)(F)C(F)F)cc3)cc2c2cc(C(=O)c3ccc(OCC4COC(C)(C)O4)cc3)c3ccccc3c21 ; CCCCC(CC)Cn1c2ccc(/C(=N/OC(C)=O)c3ccccc3OCC(F)(F)C(F)F)cc2c2cc(-c3nc(-c4ccc(OC)cc4)c(-c4ccc(OC)cc4)[nH]3)c3ccccc3c21]

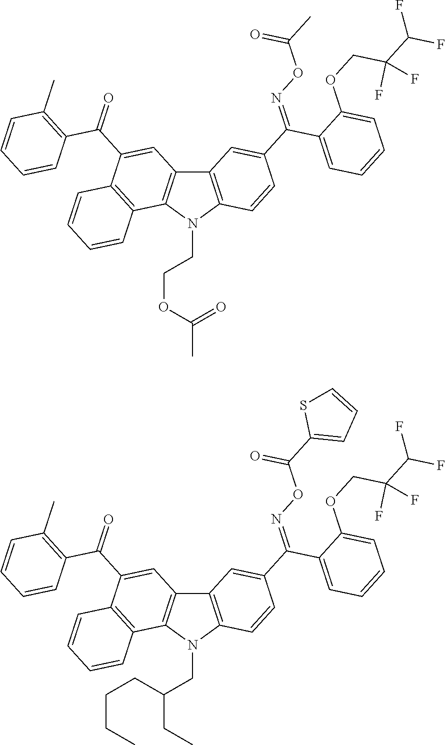 [SMILES: CC(=O)OCCn1c2ccc(/C(=N/OC(C)=O)c3ccccc3OCC(F)(F)C(F)F)cc2c2cc(C(=O)c3ccccc3C)c3ccccc3c21 ; CCCCC(CC)Cn1c2ccc(/C(=N/OC(=O)c3cccs3)c3ccccc3OCC(F)(F)C(F)F)cc2c2cc(C(=O)c3ccccc3C)c3ccccc3c21]

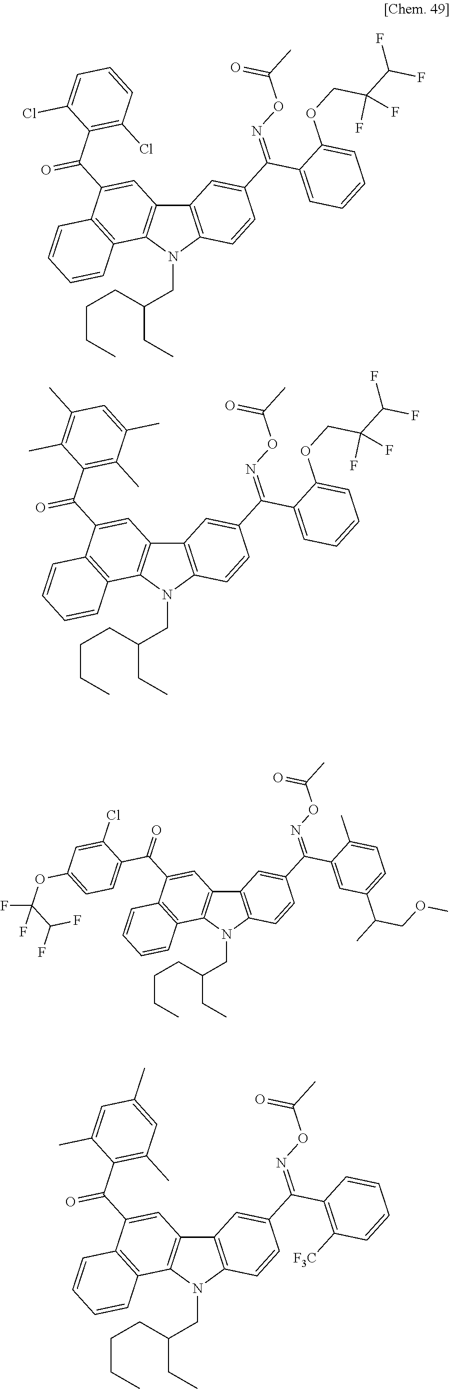

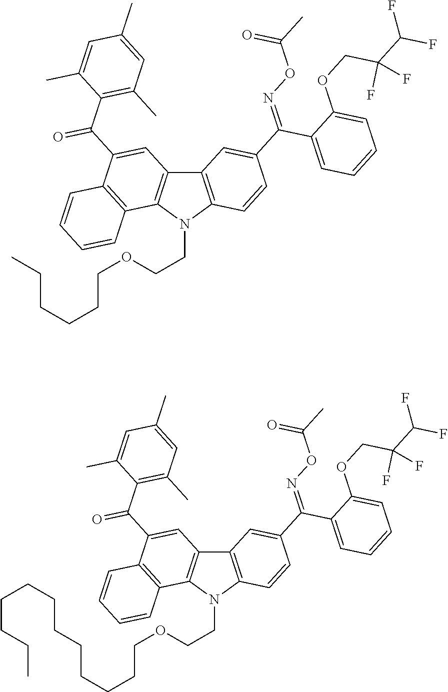 [SMILES: CCCCCCOCCn1c2ccc(/C(=N/OC(C)=O)c3ccccc3OCC(F)(F)C(F)F)cc2c2cc(C(=O)c3c(C)cc(C)cc3C)c3ccccc3c21 ; CCCCCCCCCCCCOCCn1c2ccc(/C(=N/OC(C)=O)c3ccccc3OCC(F)(F)C(F)F)cc2c2cc(C(=O)c3c(C)cc(C)cc3C)c3ccccc3c21]

Preferable specific examples of the compound represented by the formula (c2) and having the group represented by the formula (c4) as R^{c1} include the following compounds.

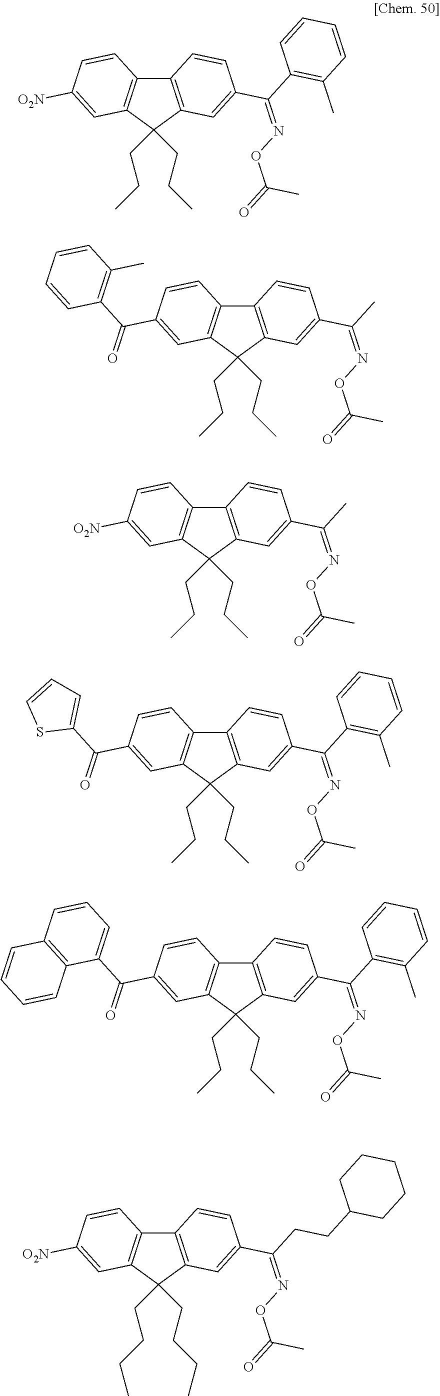

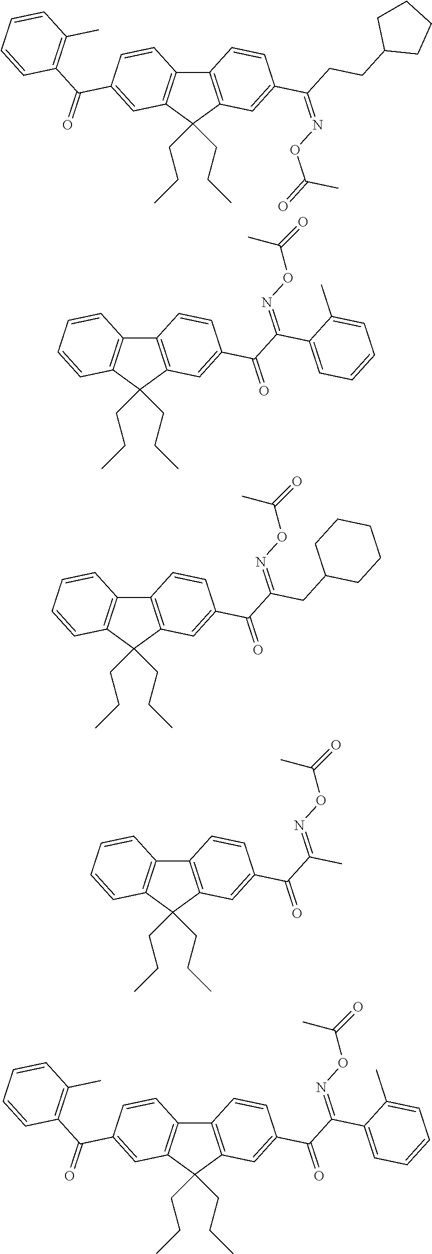

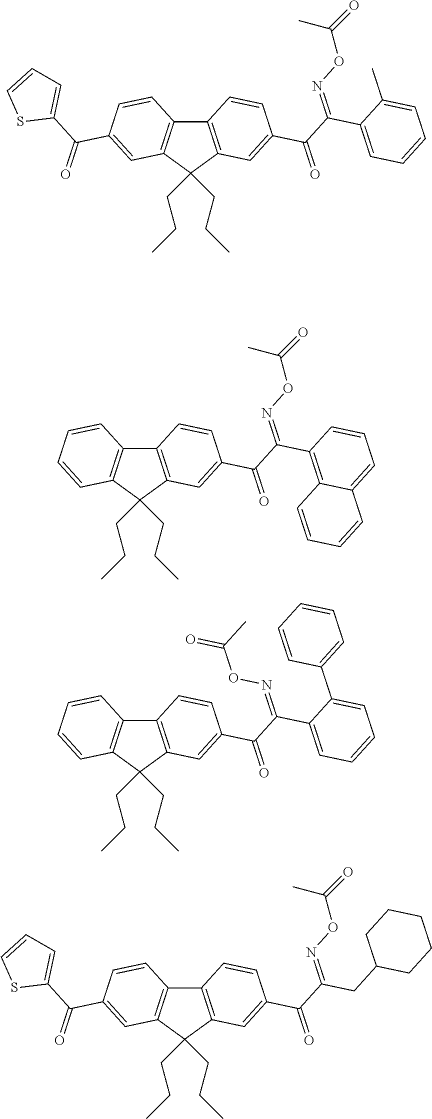 [SMILES: CCCC1(CCC)c2cc(C(=O)/C(=N/OC(C)=O)c3ccccc3C)ccc2-c2ccc(C(=O)c3cccs3)cc21 ; CCCC1(CCC)c2ccccc2-c2ccc(C(=O)/C(=N/OC(C)=O)c3cccc4ccccc34)cc21 ; CCCC1(CCC)c2ccccc2-c2ccc(C(=O)/C(=N\OC(C)=O)c3ccccc3-c3ccccc3)cc21 ; CCCC1(CCC)c2cc(C(=O)/C(CC3CCCCC3)=N/OC(C)=O)ccc2-c2ccc(C(=O)c3cccs3)cc21]

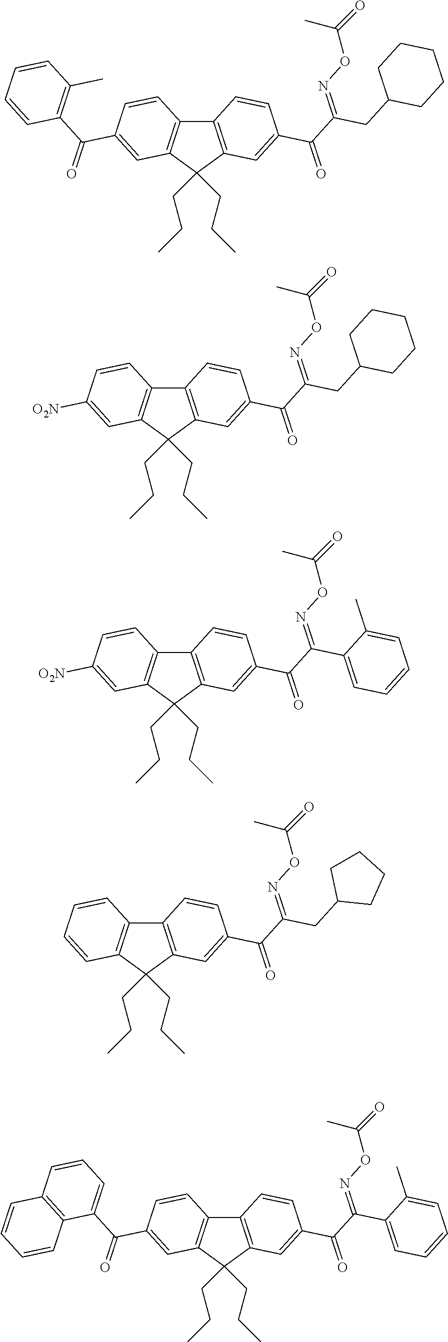 [SMILES: CCCC1(CCC)c2cc(C(=O)/C(CC3CCCCC3)=N/OC(C)=O)ccc2-c2ccc(C(=O)c3ccccc3C)cc21 ; CCCC1(CCC)c2cc(C(=O)/C(CC3CCCCC3)=N/OC(C)=O)ccc2-c2ccc([N+](=O)[O-])cc21 ; CCCC1(CCC)c2cc(C(=O)/C(=N/OC(C)=O)c3ccccc3C)ccc2-c2ccc([N+](=O)[O-])cc21 ; CCCC1(CCC)c2ccccc2-c2ccc(C(=O)/C(CC3CCCC3)=N/OC(C)=O)cc21 ; CCCC1(CCC)c2cc(C(=O)/C(=N/OC(C)=O)c3ccccc3C)ccc2-c2ccc(C(=O)c3cccc4ccccc34)cc21]

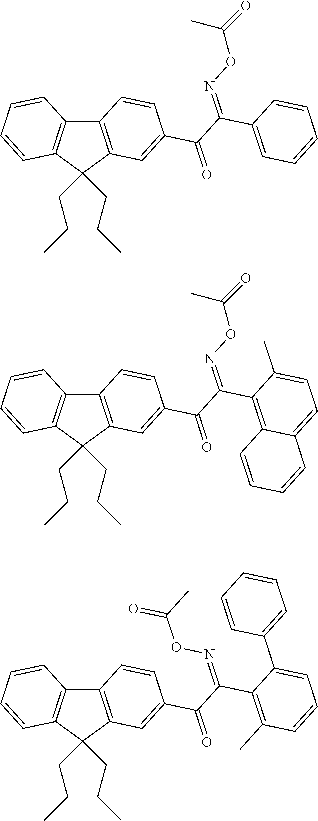 [SMILES: CCCC1(CCC)c2ccccc2-c2ccc(C(=O)/C(=N/OC(C)=O)c3ccccc3)cc21 ; CCCC1(CCC)c2ccccc2-c2ccc(C(=O)/C(=N/OC(C)=O)c3c(C)ccc4ccccc34)cc21 ; CCCC1(CCC)c2ccccc2-c2ccc(C(=O)/C(=N\OC(C)=O)c3c(C)cccc3-c3ccccc3)cc21]

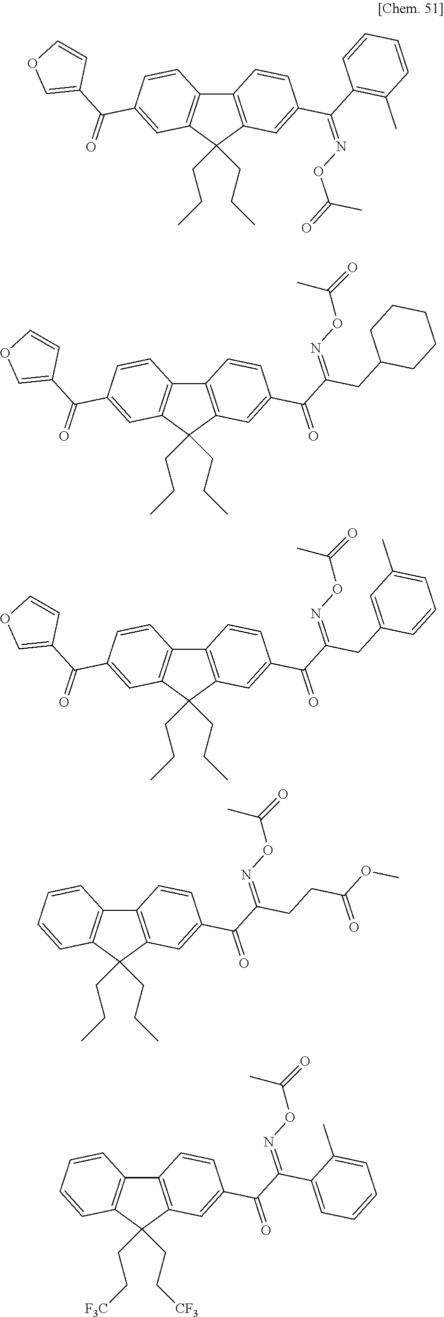

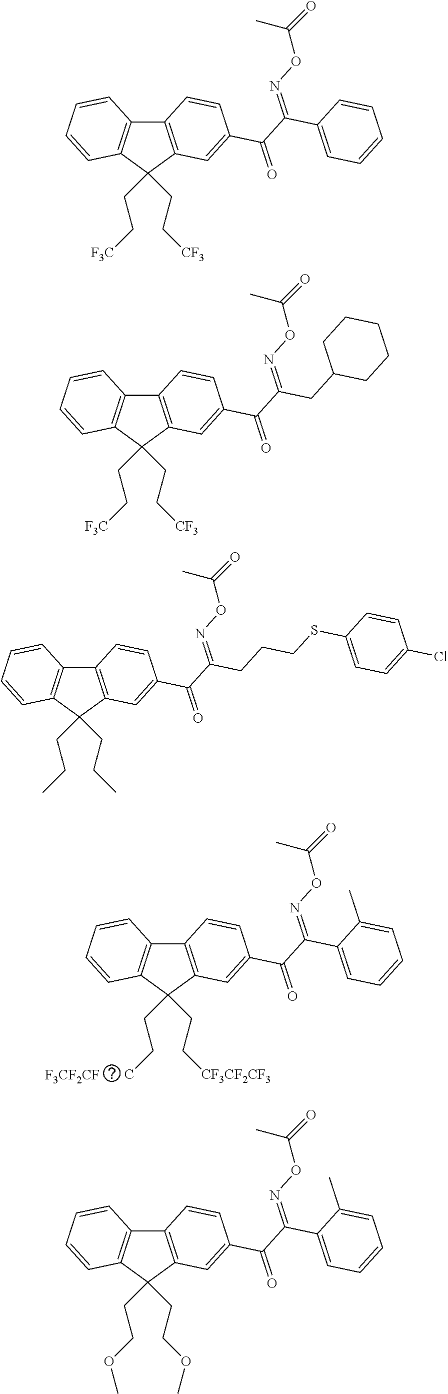

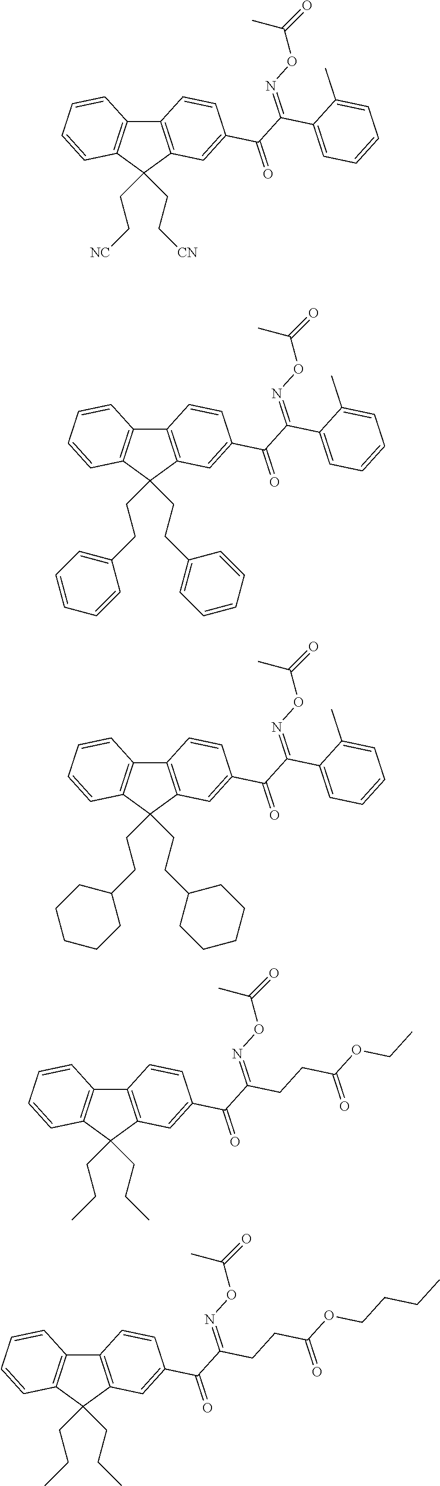

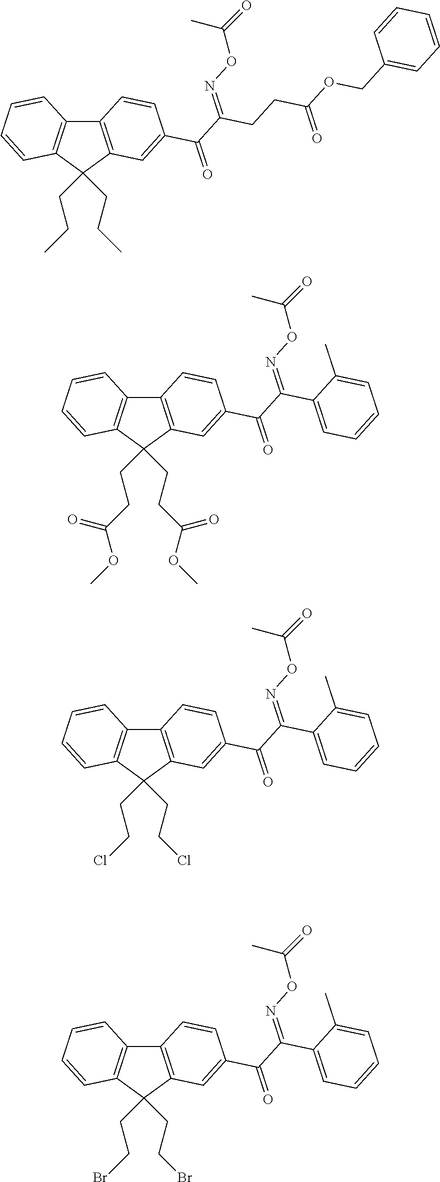 [SMILES: CCCC1(CCC)c2ccccc2-c2ccc(C(=O)/C(CCC(=O)OCc3ccccc3)=N/OC(C)=O)cc21 ; COC(=O)CCC1(CCC(=O)OC)c2ccccc2-c2ccc(C(=O)/C(=N/OC(C)=O)c3ccccc3C)cc21 ; CC(=O)O/N=C(/C(=O)c1ccc2c(c1)C(CCCl)(CCCl)c1ccccc1-2)c1ccccc1C ; CC(=O)O/N=C(/C(=O)c1ccc2c(c1)C(CCBr)(CCBr)c1ccccc1-2)c1ccccc1C]

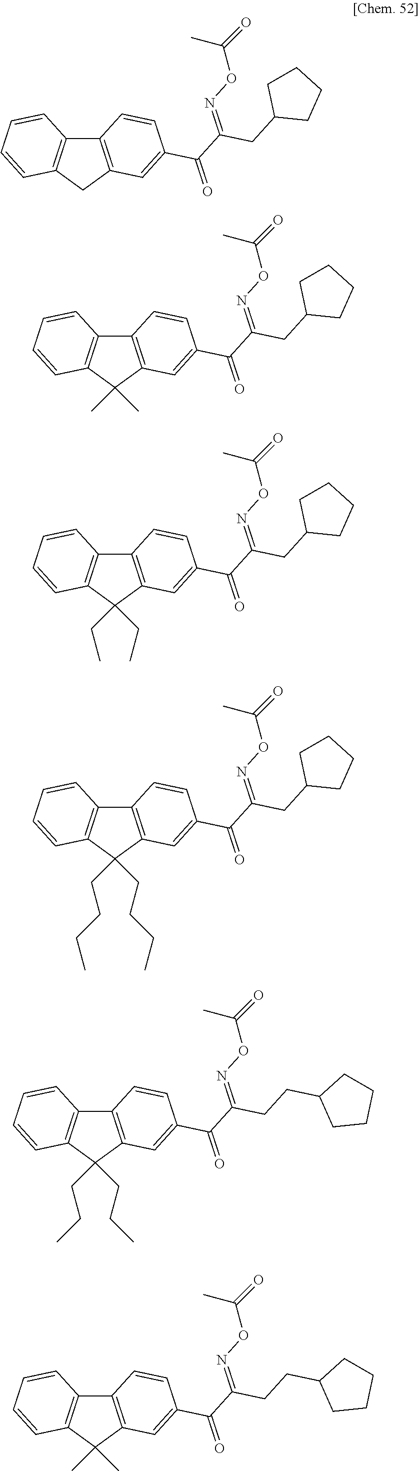

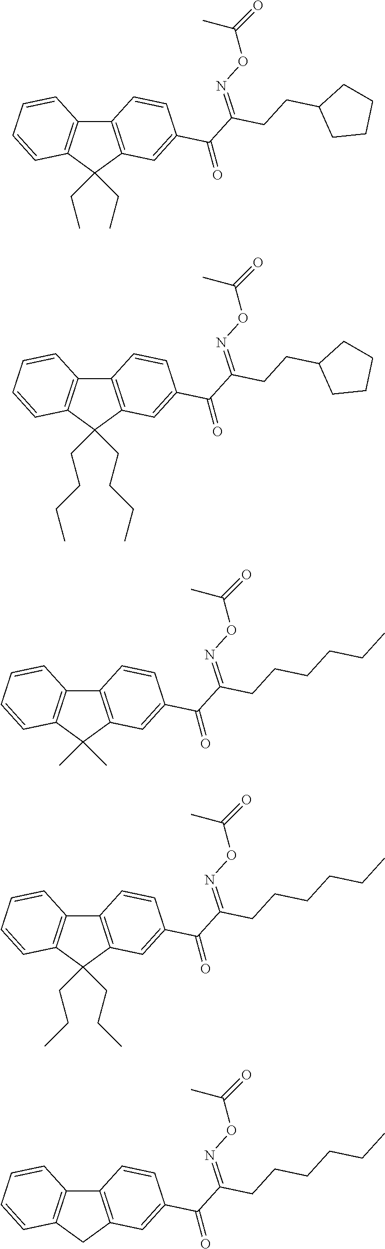 [SMILES: CCC1(CC)c2ccccc2-c2ccc(C(=O)/C(CCC3CCCC3)=N/OC(C)=O)cc21 ; CCCCC1(CCCC)c2ccccc2-c2ccc(C(=O)/C(CCC3CCCC3)=N/OC(C)=O)cc21 ; CCCCCC/C(=N\OC(C)=O)C(=O)c1ccc2c(c1)C(C)(C)c1ccccc1-2 ; CCCCCC/C(=N\OC(C)=O)C(=O)c1ccc2c(c1)C(CCC)(CCC)c1ccccc1-2 ; CCCCCC/C(=N\OC(C)=O)C(=O)c1ccc2c(c1)Cc1ccccc1-2]

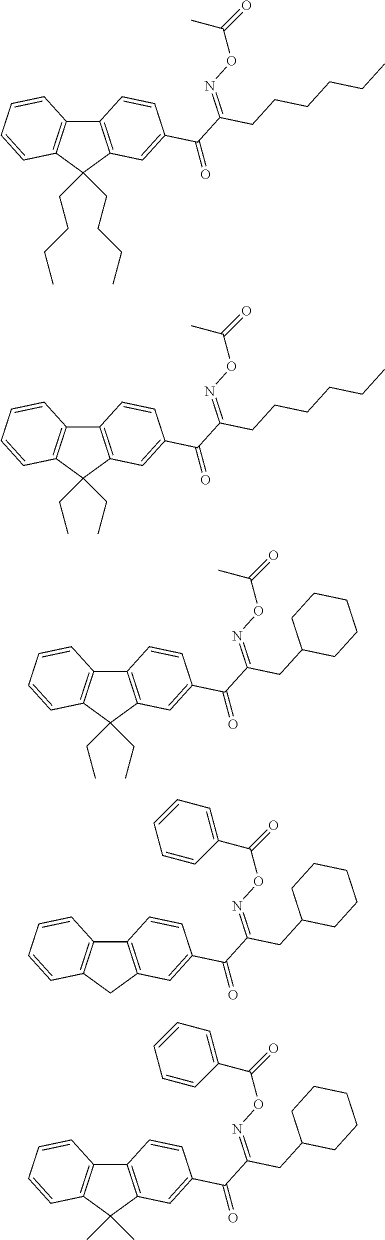

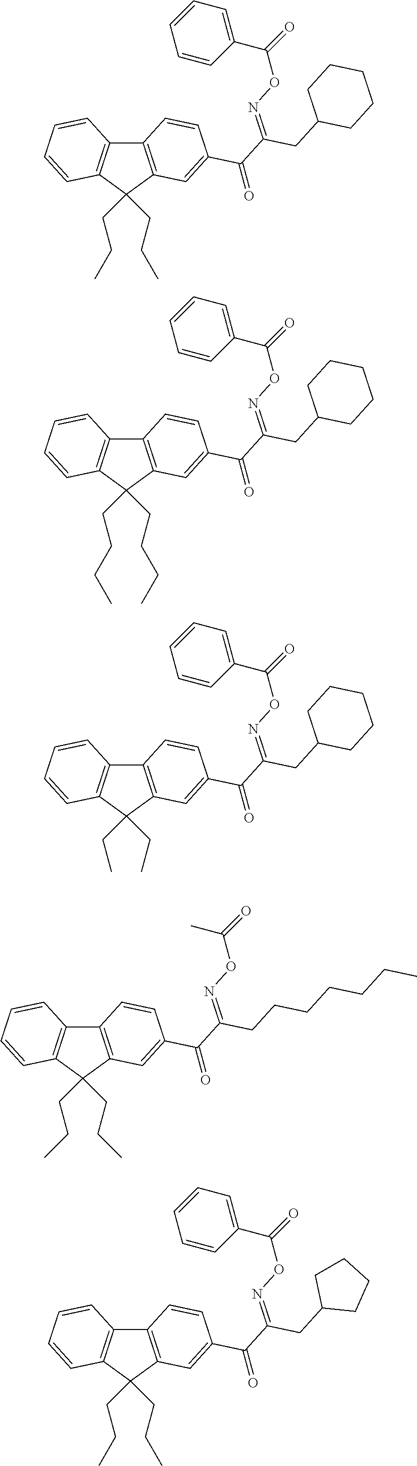

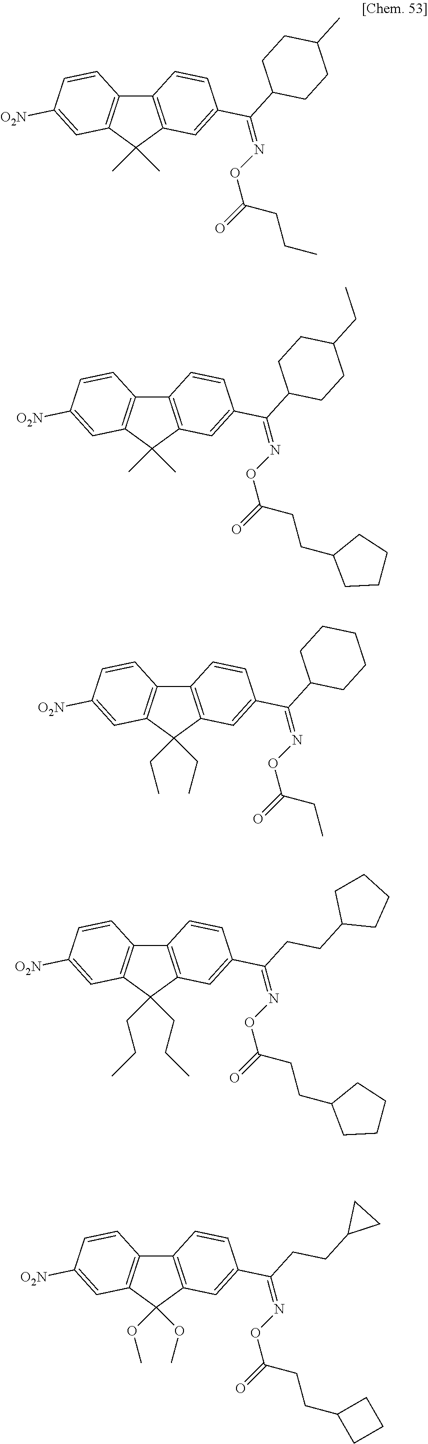

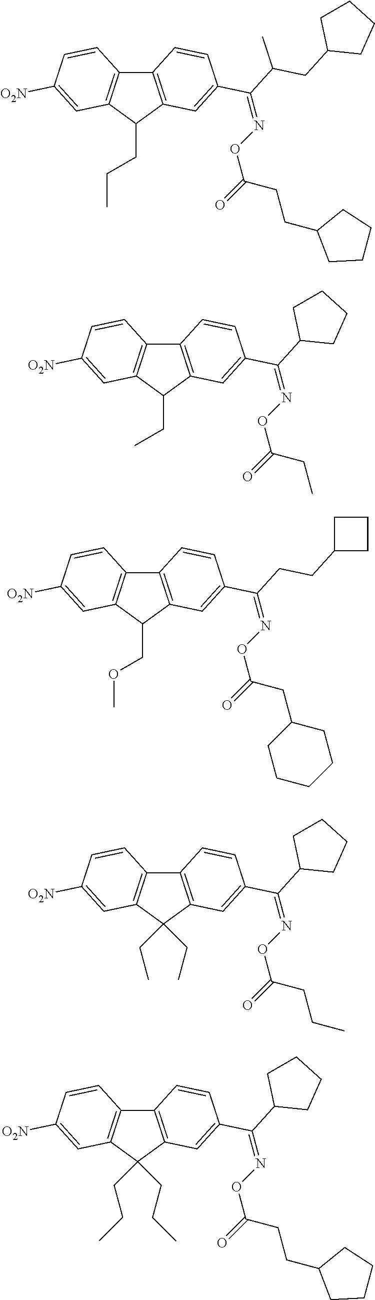

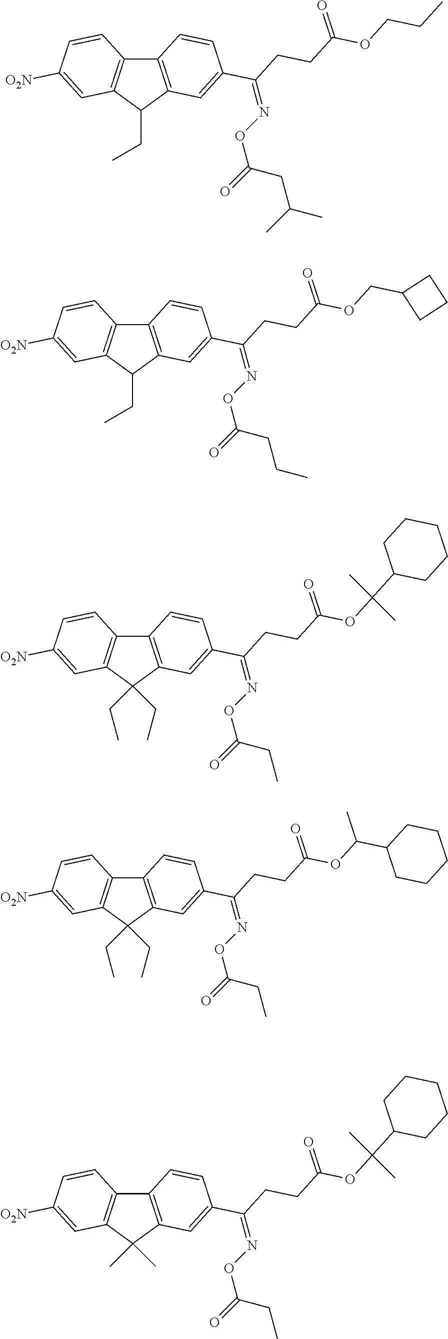 [SMILES: CCCOC(=O)CC/C(=N/OC(=O)CC(C)C)c1ccc2c(c1)C(CC)c1cc([N+](=O)[O-])ccc1-2 ; CCCC(=O)O/N=C(/CCC(=O)OCC1CCC1)c1ccc2c(c1)C(CC)c1cc([N+](=O)[O-])ccc1-2 ; CCC(=O)O/N=C(/CCC(=O)OC(C)(C)C1CCCCC1)c1ccc2c(c1)C(CC)(CC)c1cc([N+](=O)[O-])ccc1-2 ; CCC(=O)O/N=C(/CCC(=O)OC(C)C1CCCCC1)c1ccc2c(c1)C(CC)(CC)c1cc([N+](=O)[O-])ccc1-2 ; CCC(=O)O/N=C(/CCC(=O)OC(C)(C)C1CCCCC1)c1ccc2c(c1)C(C)(C)c1cc([N+](=O)[O-])ccc1-2]

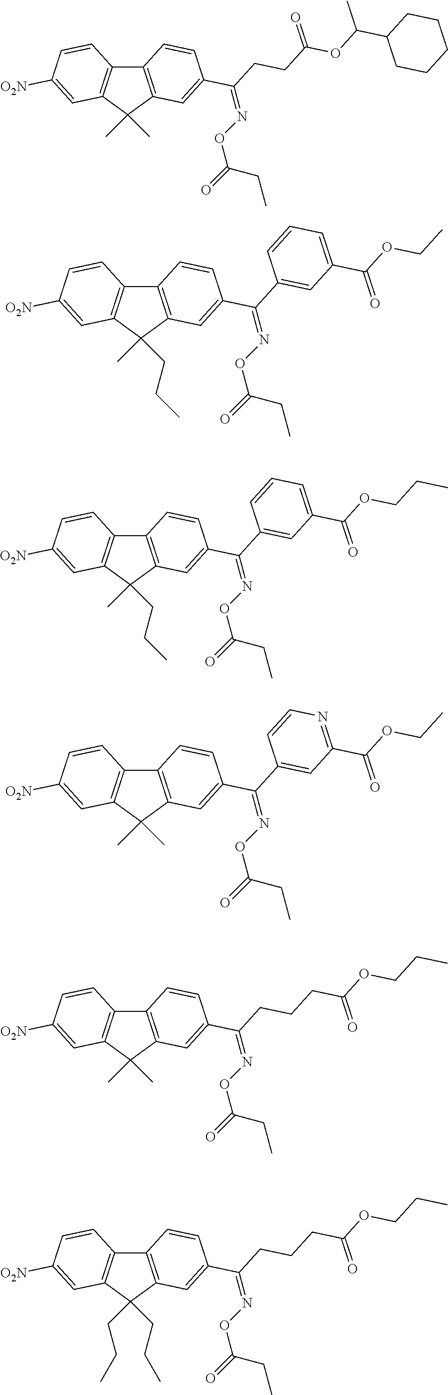

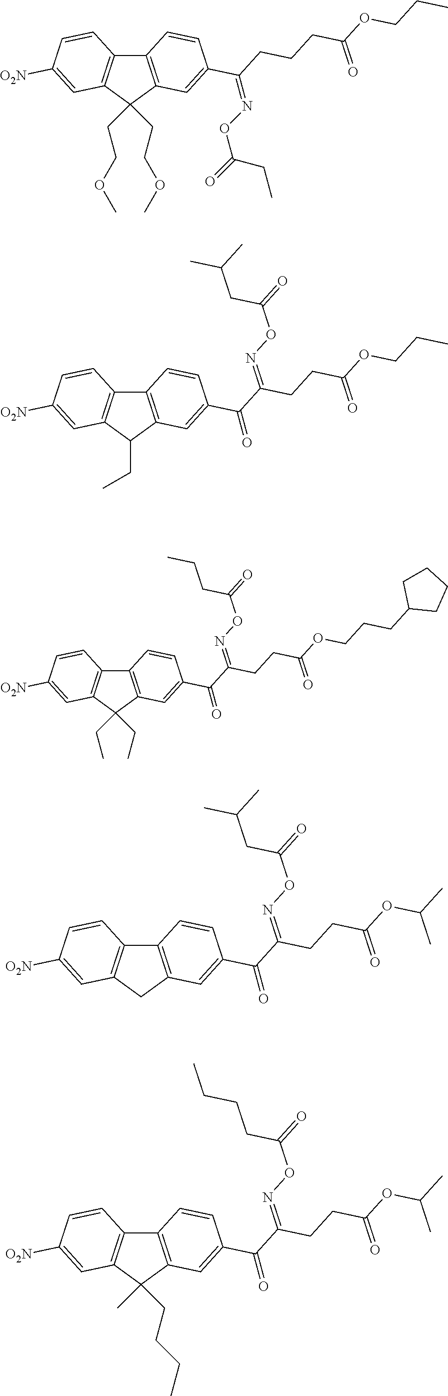 [SMILES: CCCOC(=O)CCC/C(=N/OC(=O)CC)c1ccc2c(c1)C(CCOC)(CCOC)c1cc([N+](=O)[O-])ccc1-2 ; CCCOC(=O)CC/C(=N\OC(=O)CC(C)C)C(=O)c1ccc2c(c1)C(CC)c1cc([N+](=O)[O-])ccc1-2 ; CCCC(=O)O/N=C(\CCC(=O)OCCCC1CCCC1)C(=O)c1ccc2c(c1)C(CC)(CC)c1cc([N+](=O)[O-])ccc1-2 ; CC(C)CC(=O)O/N=C(\CCC(=O)OC(C)C)C(=O)c1ccc2c(c1)Cc1cc([N+](=O)[O-])ccc1-2 ; CCCCC(=O)O/N=C(\CCC(=O)OC(C)C)C(=O)c1ccc2c(c1)C(C)(CCCC)c1cc([N+](=O)[O-])ccc1-2]

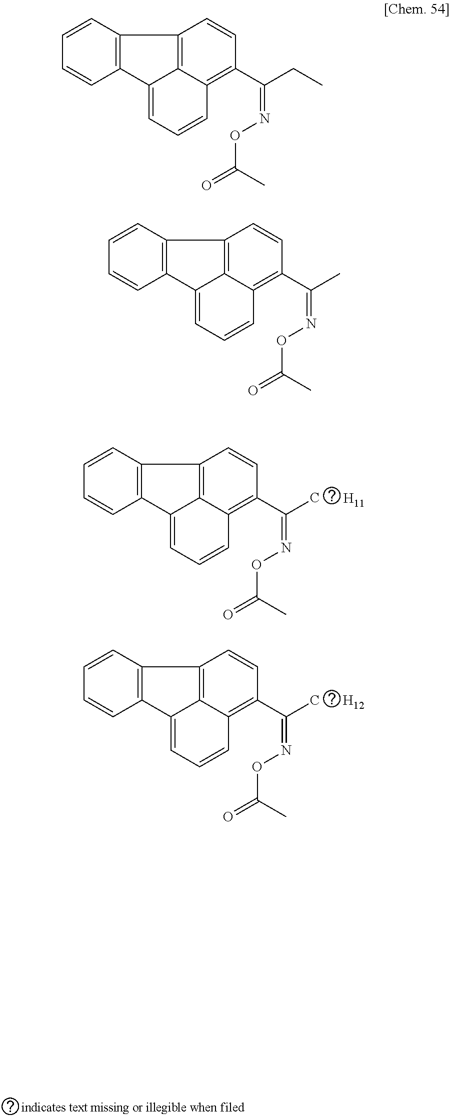

Preferable specific examples of the compound represented by the formula (c2) and having the group represented by the formula (c5) as R^{c1} include following compounds.

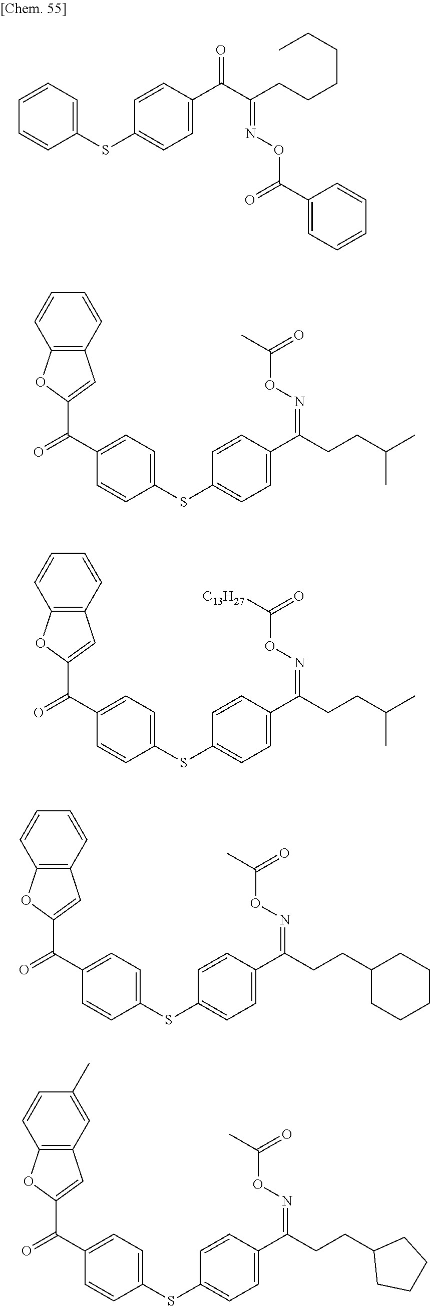

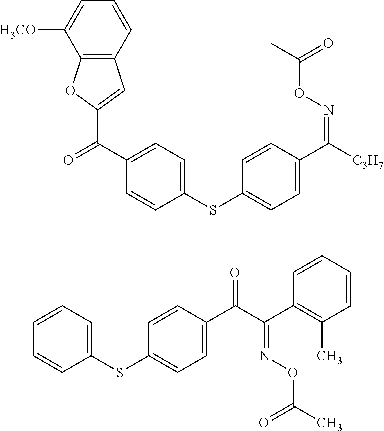

As the radical polymerization initiator (Cl), in view of good deeply curing property of the photosensitive composition, a phosphine oxide compound is also preferred. As the phosphine oxide compound, a phosphine oxide compound having a partial structure represented by the following formula (c9) is preferred.

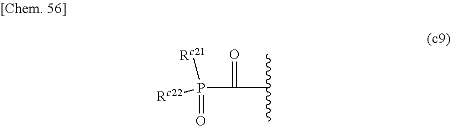

In the formula (c9), R^{c21} and R^{c22} are each independently an alkyl group, a cycloalkyl group, an aryl group, an aliphatic acyl group having 2 or more and 20 or less carbon atoms, or an aromatic acyl group having 7 or more and 20 or less carbon atoms. Provided that, both of R^{c21} and R^{c22} are the aliphatic acyl group or the aromatic aryl group.

A number of carbon atoms in the alkyl group as R^{c21} and R^{c22} is preferably 1 or more and 12 or less, more preferably 1 or more and 8 or less, and further preferably 1 or more and 4 or less. The alkyl group as R^{c21} and R^{c22} may be linear or branched. Specific examples of the alkyl group include methyl group, ethyl group, n-propyl group, isopropyl group, n-butyl group, isobutyl group, sec-butyl group, tert-butyl group, n-pentyl group, isopentyl group, tert-pentyl group, n-hexyl group, n-heptyl group, n-octyl group, 2,4,4-trimethylpentyl group, 2-ethylhexyl group, n-nonyl group, n-decyl group, n-undecyl group, and n-dodecyl group.

A number of carbon atoms in the cycloalkyl group as R^{c21} and R^{c22} is preferably 5 or more and 12 or less. Specific examples of the cycloalkyl group include cyclopentyl group, cyclohexyl group, cycloheptyl group, cyclooctyl group, cyclononyl group, cyclodecyl group, cycloundecyl group, and cyclododecyl group.

A number of carbon atoms in the aryl group as R^{e21} and R^{c22} is preferably 6 or more and 12 or less. The aryl group may have a substituent. Examples of the substituent include a halogen atom, an alkyl group having 1 or more and 4 or less carbon atoms, an alkoxy group having 1 or more and 4 or less carbon atoms, and the like. Specific examples of the aryl group include phenyl group and naphthyl group.

A number of carbon atoms in the aliphatic acyl group as R^{c21} and R^{c22} is 2 or more and 20 or less, preferably 2 or more and 12 or less, more preferably 2 or more and 8 or less, and further preferably 2 or more and 6 or less. The aliphatic acyl group may be linear or branched. Specific examples of the aliphatic acyl group include acetyl group, propionyl group, butanoyl group, pentanoyl group, hexanoyl group, heptanoyl group, octanoyl group, nonanoyl group, decanoyl group, undecanoyl group, dodecanoyl group, tridecanoyl group, tetradecanoyl group, pentadecenoyl group, hexadecanoyl group, heptadecanonyl group, octadecanoyl group, nonadecanonyl group, and icosanoyl group.

A number of carbon atoms in the aromatic acyl group as R^{c21} and R^{c22} is 7 or more and 20 or less. The aromatic acyl group may have a substituent. Examples of the substituent include a halogen atom, an alkyl group having 1 or more and 4 or less carbon atoms, an alkoxy group having 1 or more and 4 or less carbon atoms, and the like. Specific examples of the aromatic acyl group include benzoyl group, o-tolyl group, m-tolyl group, p-tolyl group, 2,6-dimethylbenzoyl group, 2,6-dimethoxybenzoyl group, 2,4,6-trimethylbenzoyl group, α-naphthoyl group, and β-naphthoyl group.

Suitable specific examples of the phosphine oxide compound having the partial structure represented by the formula (c9) include 2,4,6-trimethylbenzoyldiphenylphosphineoxide, bis(2,4,6-trimethylbenzoyl)-phenylphosphineoxide, bis(2,6-dimethoxybenzoyl)-2,4,4-trimethyl-pentylphosphineoxide, and the like. In view of deeply curing property of the photosensitive composition, it is also preferred that the phosphine oxide compound having the partial structure represented by the formula (c9) is used with α-hydroxyalkylphenone type initiator such as 2-hydroxy-2-methylpropiophenone. When the phosphine oxide compound having the partial structure represented by the formula (c9) is used with α-hydroxyalkylphenone type initiator such as 2-hydroxy-2-methylpropiophenone, a ratio of a mass of the phosphine oxide compound having the partial structure represented by the formula (c9) is preferably 20% by mass or more and 80% by mass or less, more preferably 30% by mass or more and 70% by mass or less, and further preferably 40% by mass or more and 60% by mass or less relative to a sum of the mass of both.

As a cationic polymerization initiator (C2), a conventionally known cationic polymerization initiator may be used without particular limitation. Typical examples of the cationically polymerization initiator (C2) can include onium salts. Examples of the cationic polymerization initiator (C2) include an oxonium salt, an ammonium salt, a phosphonium salt, a sulfonium salt, and an iodonium salt. A sulfonium salt and an iodonium salt are preferred, and a sulfonium salt is more preferred.

The content of the initiator (C) in the photosensitive composition is not particularly limited. The content of the initiator (C) is appropriately determined depending on the type of radically polymerizable group or cationically polymerizable group, or the type of initiator (C). The content of the initiator (C) in the photosensitive composition is preferably 0.01 parts by mass or more and 20 parts by mass or less, more preferably 0.1 parts by mass or more and 15 parts by mass or less, and even more preferably 1 part by mass or more and 10 parts by mass or less relative to 100 parts by mass of the mass of the photosensitive composition excluding the mass of the solvent (S) described later.

The photosensitive composition can be obtained by uniformly mixing and dispersing desired amount of each of the photopolymerizable compound (A), the inorganic microparticles (B), initiator (C), and optional components added as needed.

### <<Production Method of Cured Product>>

A cured product can be produced by shaping the photosensitive composition described above into a desired shape and then subjecting the photosensitive composition to curing by a method in accordance with the types and ratios of components in the photosensitive composition.

The shaping method of the photosensitive composition is not particularly limited and appropriately selected depending on the shape of the cured product. Examples of the shaping method include coating and casting into a mold. Hereinafter, a production method of a cured film will be described as a representative example of the production method of the cured product.

First, the photosensitive composition is coated on a desired substrate to form a coating film. Then, as needed, the solvent (S) is at least partially removed from the coating film to form a coating film.

The method of coating the photosensitive composition on the substrate is not particularly limited. The coating film can be formed by coating the photosensitive composition on the substrate such that a desired film thickness is achieved, using a contact transfer-type applicator such as a roll coater, a reverse coater, a bar coater, or a slit coater, or a non-contact type applicator such as a spinner (a rotary applicator) or a curtain flow coater, for example. In addition, printing methods such as screen printing and inkjet printing can also be applied to form the coating film. As described above, the aforementioned photosensitive composition is not likely to dry rapidly, and thicken or solidify in the inkjet head. Therefore, by using the aforementioned photosensitive composition, applying by inkjet printing method can be performed well.

After applying the photosensitive composition on the substrate, as needed, the solvent (S) is preferably removed at least partially from the coating film by baking the coating film. The baking temperature is appropriately determined in consideration of the boiling point of the solvent (S) and the like. The baking may be carried out at a low temperature under reduced pressure conditions.

The method of baking is not particularly limited, and examples thereof include a method in which the coating film is dried using a hot plate at a temperature of 80° C. or higher and 150° C. or lower and preferably 85° C. or higher and 120° C. or lower, for 60 seconds or longer and 500 seconds or shorter.

The film thickness of the coating film formed as described above is not particularly limited. The film thickness of the coating film is appropriately determined depending on the applications of the cured film. The film thickness of the coating film is typically appropriately adjusted such that a cured film to be formed has a film thickness of preferably 0.1 μm or more and 10 μm or less and more preferably 0.2 μm or more and 5 μm or less.

After a coating film is formed by the above method, the coating film is subjected to light exposure to thereby enable a cured film to be obtained.

A condition for light exposure to the coating film is not particularly limited as long as curing proceeds well. Light exposure is carried out by irradiation with, for example, active energy rays such as ultraviolet rays and excimer laser light. The dose of energy used in the irradiation is not particularly limited, and examples thereof include a dose of 30 mJ/cm² or more and 5000 mJ/cm² or less. After the light exposure, the coating film subjected to light exposure may be baked in the same manner as the heating after coating.

### EXAMPLES

Hereinafter, the present invention is described in more detail by way of Examples, but the present invention is not limited to these Examples.

### Synthesis Example 1

4,4′-thiobisbenzenethiol (20.0 g, 0.080 mol) and 100 mL of 20% aqueous sodium hydroxide solution were added to a reaction vessel with 300 mL capacity. After nitrogen replacement in the reaction vessel, contents in the reaction vessel were stirred at an internal temperature of 60° C. for 30 minutes. Thereafter, 1-chloro-3-ethoxy-2-propanol (25.5 g, 0.186 mol) was dropped into the reaction vessel, and reaction was carried out at internal temperature of 90° C. for 4 hours. Subsequently, the reaction solution was cooled to room temperature with stirring. Solid precipitated by cooling was filtered out, and washed with water. The resulting solid was dried under reduced pressure to give 36.2 g (Yield 99%) of compound IM1. Subsequently, compound IM1 (36.2 g, 0.079 mol), triethylamine (19.3 g, 0.191 mol), hydroquinone (0.88 g, 0.008 mol) and 360 mL of tetrahydrofuran was mixed in a reaction vessel with 500 mL capacity. Obtained solution was cooled in an ice bath. Acryloyl chloride (17.3 g, 0.191 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 24.5 g (Yield 55%) of compound 1 as colorless liquid. Measurement result of ¹H-NMR of compound 1 is shown below.

¹H-NMR (DMSO): 1.05 (t, 6H), 2.95 (dd, 4H), 3.65 (dd, 4H), 3.88 (q, 4H), 5.17 (m, 2H), 5.83 (dd, 2H), 6.12 (dd, 2H), 6.41 (dd, 2H), 7.16 (d, 4H), 7.28 (d, 4H)

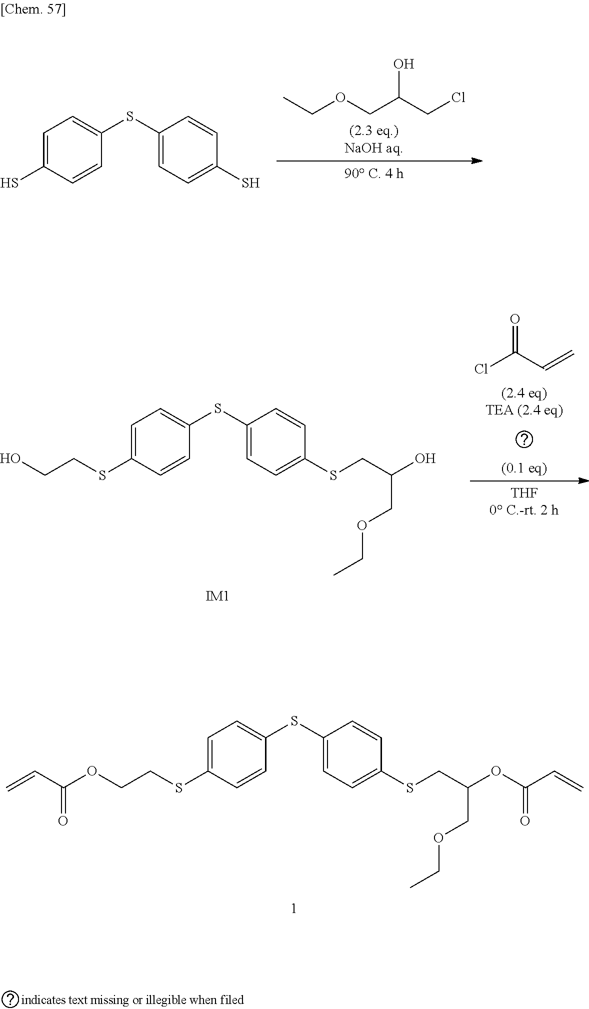

### Synthesis Example 2

4,4′-thiobisbenzenethiol (20.0 g, 0.080 mol) and 100 mL of 20% aqueous sodium hydroxide solution were added to a reaction vessel with 300 mL capacity. After nitrogen replacement in the reaction vessel. contents in the reaction vessel were stirred at an internal temperature of 60° C. for 30 minutes. Thereafter, ethylene glycol mono-2-chloro ether (23.2 g, 0.186 mol) was dropped into the reaction vessel, and reaction was carried out at internal temperature of 90° C. for 4 hours. Subsequently, the reaction solution was cooled to room temperature with stirring. Solid precipitated by cooling was filtered out, and washed with water. The resulting solid was dried under reduced pressure to give 33.8 g (Yield 99%) of compound IM2. Subsequently, compound IM2 (33.8 g, 0.079 mol), triethylamine (19.2 g, 0.190 mol), hydroquinone (0.87 g, 0.008 mol) and 340 mL of tetrahydrofuran was mixed in a reaction vessel with 500 mL capacity. Obtained solution was cooled in an ice bath. Acryloyl chloride (17.2 g, 0.190 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 22.1 g (Yield 52%) of compound 2 as colorless liquid. Measurement result of ¹H-NMR of compound 2 is shown below. ¹H-NMR (DMSO): 3.27 (t, 4H), 3.63 (t, 4H), 3.77 (t, 4H), 4.27 (t, 4H), 5.80 (dd, 2H), 6.15 (dd, 2H), 6.41 (dd, 2H), 7.20 (d, 4H), 7.28 (d, 4H)

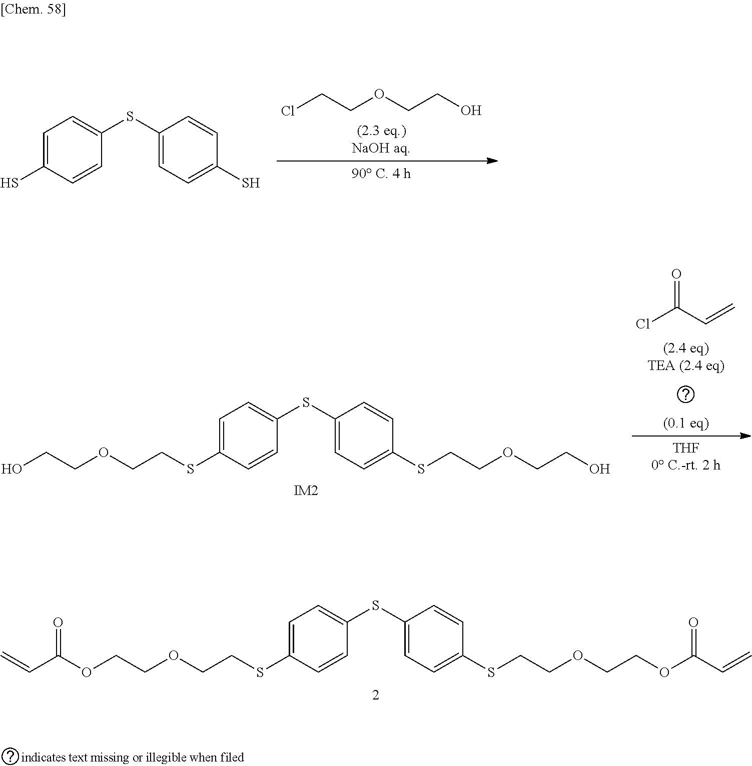

### Synthesis Example 3

4,4′-thiobisbenzenethiol (20.0 g, 0.080 mol) and 100 mL of 20% aqueous sodium hydroxide solution were added to a reaction vessel with 300 mL capacity. After nitrogen replacement in the reaction vessel, contents in the reaction vessel were stirred at an internal temperature of 60° C. for 30 minutes. Thereafter, 2-[2-(2-chloroethoxy)ethoxy]ethanol (31.4 g, 0.186 mol) was dropped into the reaction vessel, and reaction was carried out at internal temperature of 90° C. for 4 hours. Subsequently, the reaction solution was cooled to room temperature with stirring. After adding water to the cooled reaction solution, toluene was further added to the reaction solution, and the reaction product was extracted in toluene. The solvent was removed from the separated toluene layer, and residues after removing the solvent was purified with silicagel chromatography to obtain 34.1 g (Yield 83%) of compound IM3. Subsequently, compound IM3 (34.1 g, 0.079 mol), triethylamine (16.1 g, 0.159 mol), hydroquinone (0.73 g, 0.007 mol) and 340 mL of tetrahydrofuran was mixed in a reaction vessel with 500 mL capacity. Obtained solution was cooled in an ice bath. Acryloyl chloride (14.4 g, 0.159 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 18.9 g (Yield 46%) of compound 3 as colorless liquid. Measurement result of ¹H-NMR of compound 3 is shown below.

¹H-NMR (DMSO): 3.27 (t, 4H), 3.50 (s, 8H), 3.63 (t, 4H), 3.80 (t, 4H), 4.30 (t, 4H), 5.80 (dd, 2H), 6.15 (dd, 2H), 6.41 (dd, 2H), 7.20 (d, 4H), 7.28 (d, 4H)

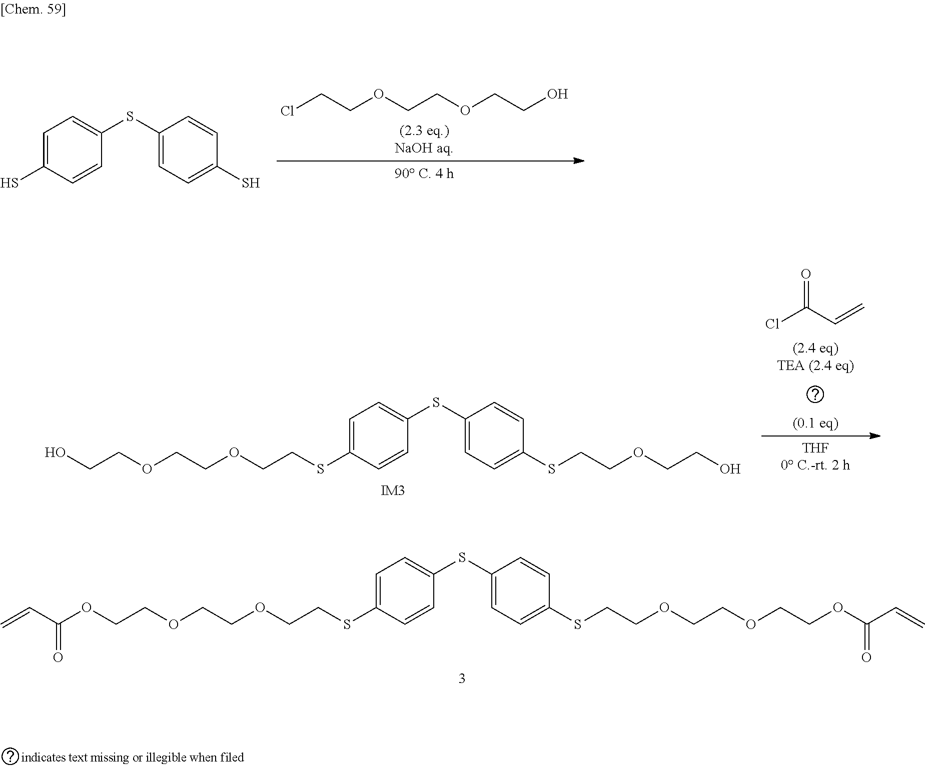

### Synthesis Example 4

4,4′-thiobisbenzenethiol (20.0 g, 0.080 mol) and 100 mL of 20% aqueous sodium hydroxide solution were added to a reaction vessel with 300 mL capacity. After nitrogen replacement in the reaction vessel, contents in the reaction vessel were stirred at an internal temperature of 60° C. for 30 minutes. Thereafter, 3-(3-chloropropoxy)propane-1-ol (28.5 g, 0.186 mol) was dropped into the reaction vessel, and reaction was carried out at internal temperature of 90° C. for 4 hours. Subsequently, the reaction solution was cooled to room temperature with stirring. After adding water to the cooled reaction solution, toluene was further added to the reaction solution, and the reaction product was extracted in toluene. The solvent was removed from the separated toluene layer, and residues after removing the solvent was purified with silicagel chromatography to obtain 36.5 g (Yield 98%) of compound IM4. Subsequently, compound IM4 (36.5 g, 0.073 mol), triethylamine (17.8 g, 0.176 mol), hydroquinone (0.81 g, 0.007 mol) and 360 mL of tetrahydrofuran was mixed in a reaction vessel with 500 mL capacity. Obtained solution was cooled in an ice bath. Acryloyl chloride (15.9 g, 0.176 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 22.6 g (Yield 54%) of compound 4 as colorless liquid. Measurement result of ¹H-NMR of compound 4 is shown below.

¹H-NMR (DMSO): 1.86 (m, 8H), 2.94 (t, 4H), 3.35 (t, 8H), 4.20 (t, 4H), 5.80 (dd, 2H), 6.15 (dd, 2H), 6.41 (dd, 2H), 7.20 (d, 4H), 7.28 (d, 4H)

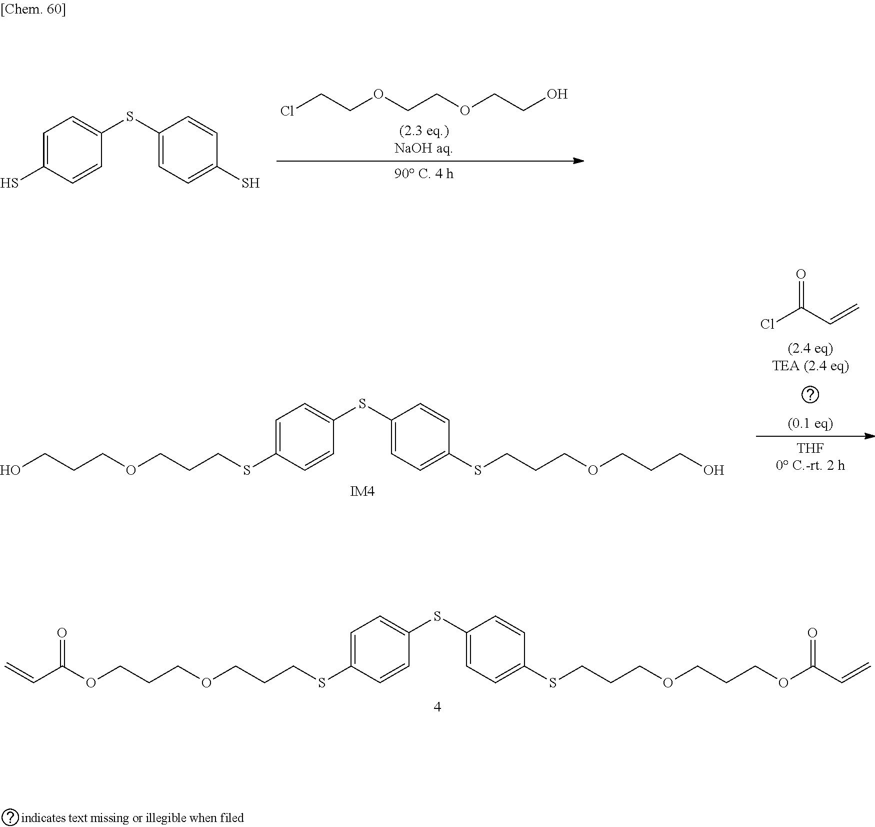

### Synthesis Example 5

4,4′-dihydroxydiphenylsulfide (20.0 g, 0.092 mol), sodium bicarbonate (38.0 g, 0.275 mol), ethylene glycol mono-2-chloroethyl ether (27.4 g, 0.220 mol), and 200 mL of dimethylformamide were added to a reaction vessel with 300 mL capacity. After nitrogen replacement in the reaction vessel, the reaction was carried out by stirring the reaction mixture at an internal temperature of 90° C. for 10 hours. Subsequently, the reaction solution was cooled to room temperature with stirring. After adding water to the cooled reaction solution, toluene was further added to the reaction solution, and the reaction product was extracted in toluene. The solvent was removed from the separated toluene layer, and residues after removing the solvent was purified with silicagel chromatography to obtain 42.3 g (Yield 86%) of compound IM5. Subsequently, compound IM5 (42.3 g, 0.079 mol), triethylamine (19.2 g, 0.190 mol), hydroquinone (0.87 g, 0.008 mol) and 420 mL of tetrahydrofuran was mixed in a reaction vessel with 300 mL capacity. Obtained solution was cooled in an ice bath. Acryloyl chloride (17.2 g, 0.190 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 22.6 g (Yield 53%) of compound 5 as colorless liquid. Measurement result of ¹H-NMR of compound 5 is shown below.

¹H-NMR (DMSO): 3.63 (t, 4H), 3.77 (t, 4H), 4.31 (m, 8H), 5.80 (dd, 2H), 6.15 (dd, 2H), 6.41 (dd, 2H), 7.20 (d, 4H), 7.28 (d, 4H)

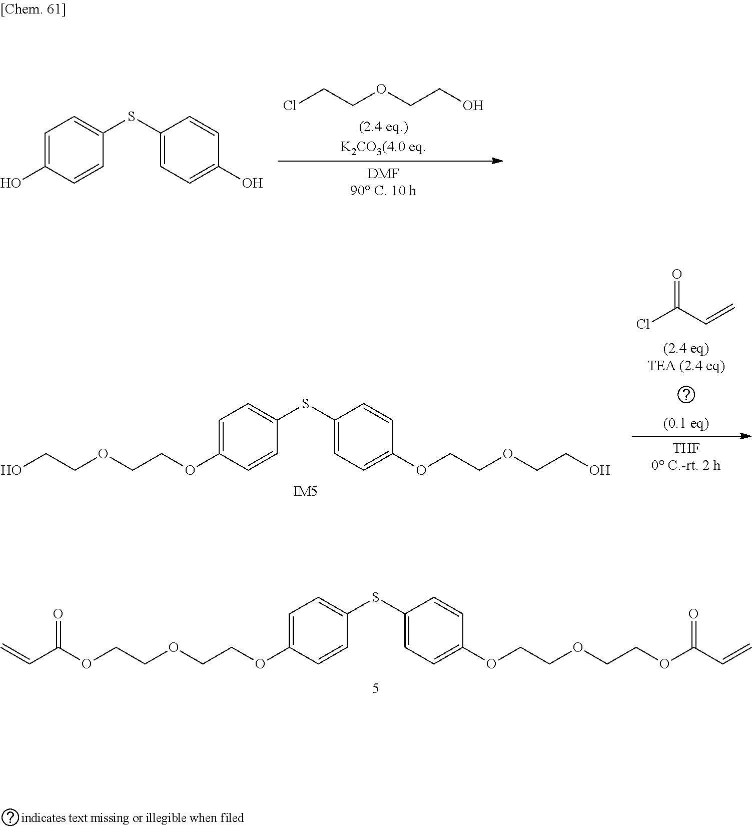

### Synthesis Example 6

4,4′-dihydroxydiphenylsulfide (20.0 g, 0.092 mol), potassium bicarbonate (38.0 g, 0.275 mol), 2-[2-(2-chloroethoxy)ethoxy]ethanol (37.1 g, 0.220 mol), and 200 mL of dimethylformamide were added to a reaction vessel with 300 mL capacity. After nitrogen replacement in the reaction vessel, the reaction was carried out by stirring the reaction mixture at an internal temperature of 90° C. for 10 hours. Subsequently, the reaction solution was cooled to room temperature with stirring. After adding water to the cooled reaction solution, toluene was further added to the reaction solution, and the reaction product was extracted in toluene. The solvent was removed from the separated toluene layer, and residues after removing the solvent was purified with silicagel chromatography to obtain 35.7 g (Yield 76%) of compound IM6. Subsequently, compound IM6 (35.7 g, 0.069 mol), triethylamine (16.8 g, 0.190 mol), hydroquinone (0.76 g, 0.007 mol) and 360 mL of tetrahydrofuran was mixed in a reaction vessel with 300 mL capacity. Obtained solution was cooled in an ice bath. Acryloyl chloride (15.1 g, 0.166 mol) was added dropwise to the cooled solution while keeping the internal temperature 5° C. or lower. Solution to which acryloyl chloride was added was stirred at room temperature for 2 hours, and precipitated salt was filtrated. From the resulting filtrate, the solvent was removed with an evaporator to obtain a viscous liquid. The obtained viscous liquid was purified with silicagel chromatography to obtain 24.2 g (Yield 56%) of compound 6 as colorless liquid. Measurement result of ¹H-NMR of compound 6 is shown below.

¹H-NMR (DMSO): 3.52 (s, 8H), 3.63 (t, 4H), 3.70 (t, 4H), 4.30 (m, 8H), 5.80 (dd, 2H), 6.15 (dd, 2H), 6.41 (dd, 2H), 7.20 (d, 4H), 7.28 (d, 4H)

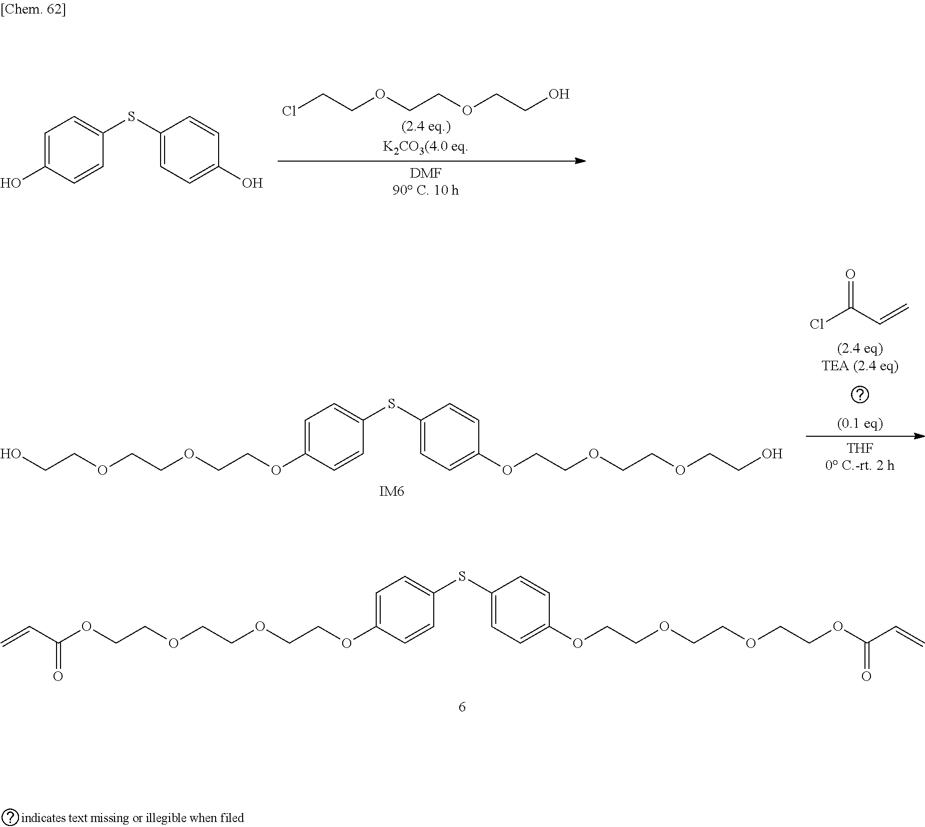

### Examples 1 to 12, Comparative Example 1, and Comparative Example 2

Simulated tests on heating of the photosensitive compositions were carried out using the compounds 1 to 6 obtained in the Synthesis Examples 1 to 6, and 2-(2-acryloyloxyethyl)oxybiphenyl as comparative compound. It should be noted that the inorganic microparticles (B) and the initiator (C) have no effect on the weight loss by heating at 110° C. Specifically, a solution including the photopolymerizable compound (A) was obtained by dissolving 150 mg of the photopolymerizable compound (A) of type shown in Table 1 in the solvent (S) of type shown in table 1 so that a solid content was 10% by mass. As the solvent (S), following S-1 and S-2 were used.
S-1: propylene glycol monomethyl ether acetate
S-2: HO—(CH₂CH₂CH₂—O)₂—CH₃ (dipropylene glycol monomethyl ether)

The obtained solution was dropped onto a glass substrate which was weighed, and the glass substrate was heated at 110° C. for 10 minutes, thereafter, the glass substrate was weighed again. Mass loss of the photopolymerizable compound (A) by heating was calculated from mass of the glass substrate before heating and mass of the glass substrate after heating. Ratio of loss of mass caused by heating the photopolymerizable compound (A) was calculated based on the mass loss of the photopolymerizable compound (A) by heating. Values of the ratio of loss are shown in Table 1.

TABLE 1

| | Photopolymerizable | Solvent | |
|---|---|---|---|
| | compound (A) | (S) | Ratio of loss of mass |
| Example 1 | Compound 1 | S-1 | 2% by mass |
| Example 2 | Compound 1 | S-2 | 3% by mass |
| Example 3 | Compound 2 | S-1 | Less than 1% by mass |
| Example 4 | Compound 2 | S-2 | Less than 1% by mass |
| Example 5 | Compound 3 | S-1 | Less than 1% by mass |
| Example 6 | Compound 3 | S-2 | Less than 1% by mass |
| Example 7 | Compound 4 | S-1 | Less than 1% by mass |
| Example 8 | Compound 4 | S-2 | Less than 1% by mass |
| Example 9 | Compound 5 | S-1 | Less than 1% by mass |
| Example 10 | Compound 5 | S-2 | Less than 1% by mass |
| Example 11 | Compound 6 | S-1 | Less than 1% by mass |
| Example 12 | Compound 6 | S-2 | Less than 1% by mass |
| Comparative | Comparative | S-1 | 12% by mass |
| Example 1 | Compound | | |
| Comparative | Comparative | S-2 | 18% by mass |
| Example 2 | Compound | | |

Comparison of Examples 1 to 12 with Comparative Example 1 and Comparative Example 2 shows that weight loss of components other than the solvent (S) by heating was significantly suppressed in the composition including the photopolymerizable compound (A) having the structure corresponding to the formula (A1), compared to the composition including the photopolymerizable compound not corresponding to the formula (A1).

### Examples 13 to 60, and Comparative Examples 3 to 10

5 parts by mass of the photopolymerizable compound (A) of type shown in Tables 2 and 3, 20 parts by mass of the inorganic microparticles (B) consisting of a material shown in Tables 2 and 3, and 0.5 parts by mass of bis(2,4,6-trimethylbenzoyl)-phenylphosphinoxide as the initiator (C) were dissolved and dispersed in the solvent (S) of type shown in Tables 2 and 3 so that the solid content was 10% by mass to obtain the photosensitive compositions of Examples 13 to 60 and Comparative Examples 3 to 10. Average particle diameters of gold microparticles, platinum microparticles, zirconium oxide microparticles, and titanium dioxide microparticles used as the inorganic microparticles (B) were all 10 nm. As the zirconium oxide microparticles and the titanium dioxide microparticles, surface treated zirconium oxide microparticles and surface treated titanium dioxide microparticles were used. The resulting photosensitive composition was placed in a thermostatic apparatus at 40° C. for 3 months, and the dispersion of inorganic microparticles in the photosensitive composition was visually confirmed after 1 month and after 3 months to evaluate the dispersion stability of the inorganic microparticles (B) in the photosensitive composition according to the following criteria.
O: Isolation of precipitation of the inorganic microparticles was not observed.
Δ: Slight isolation or precipitation of the inorganic microparticles (B) was observed.
X: Significant isolation or precipitation of the inorganic microparticles (B) was observed.

TABLE 2

| | | Material | | | |
|---|---|---|---|---|---|
| | Photo- | of | | | |
| | poly- | inorganic | | Dispersion | |
| | merizable | micro- | | stability | |
| | compound | particles | Solvent | 1 | 3 |
| | (A) | (B) | (S) | month | months |
| Example 13 | Compound 1 | Au | S-1 | Δ | Δ |
| Example 14 | Compound 1 | Au | S-2 | Δ | Δ |
| Example 15 | Compound 1 | Pt | S-1 | Δ | Δ |
| Example 16 | Compound 1 | Pt | S-2 | Δ | Δ |
| Example 17 | Compound 1 | ZrO₂ | S-1 | ○ | Δ |
| Example 18 | Compound 1 | ZrO₂ | S-2 | ○ | Δ |
| Example 19 | Compound 1 | TiO₂ | S-1 | ○ | Δ |
| Example 20 | Compound 1 | TiO₂ | S-2 | ○ | Δ |
| Example 21 | Compound 2 | Au | S-1 | ○ | Δ |
| Example 22 | Compound 2 | Au | S-2 | ○ | Δ |
| Example 23 | Compound 2 | Pt | S-1 | ○ | Δ |
| Example 24 | Compound 2 | Pt | S-2 | ○ | Δ |
| Example 25 | Compound 2 | ZrO₂ | S-1 | ○ | ○ |
| Example 26 | Compound 2 | ZrO₂ | S-2 | ○ | ○ |
| Example 27 | Compound 2 | TiO₂ | S-1 | ○ | ○ |
| Example 28 | Compound 2 | TiO₂ | S-2 | ○ | ○ |
| Example 29 | Compound 3 | Au | S-1 | ○ | Δ |
| Example 30 | Compound 3 | Au | S-2 | ○ | ○ |
| Example 31 | Compound 3 | Pt | S-1 | ○ | Δ |
| Example 32 | Compound 3 | Pt | S-2 | ○ | ○ |
| Example 33 | Compound 3 | ZrO₂ | S-1 | ○ | ○ |
| Example 34 | Compound 3 | ZrO₂ | S-2 | ○ | ○ |
| Example 35 | Compound 3 | TiO₂ | S-1 | ○ | ○ |
| Example 36 | Compound 3 | TiO₂ | S-2 | ○ | ○ |
| Example 37 | Compound 4 | Au | S-1 | ○ | Δ |
| Example 38 | Compound 4 | Au | S-2 | ○ | Δ |
| Example 39 | Compound 4 | Pt | S-1 | ○ | Δ |
| Example 40 | Compound 4 | Pt | S-2 | ○ | Δ |
| Example 41 | Compound 4 | ZrO₂ | S-1 | ○ | Δ |
| Example 42 | Compound 4 | ZrO₂ | S-2 | ○ | Δ |
| Example 43 | Compound 4 | TiO₂ | S-1 | ○ | Δ |
| Example 44 | Compound 4 | TiO₂ | S-2 | ○ | Δ |
| Example 45 | Compound 5 | Au | S-1 | Δ | Δ |
| Example 46 | Compound 5 | Au | S-2 | Δ | Δ |
| Example 47 | Compound 5 | Pt | S-1 | Δ | Δ |
| Example 48 | Compound 5 | Pt | S-2 | Δ | Δ |
| Example 49 | Compound 5 | ZrO₂ | S-1 | ○ | ○ |
| Example 50 | Compound 5 | ZrO₂ | S-2 | ○ | ○ |
| Example 51 | Compound 5 | TiO₂ | S-1 | ○ | ○ |
| Example 52 | Compound 5 | TiO₂ | S-2 | ○ | ○ |
| Example 53 | Compound 6 | Au | S-1 | ○ | ○ |
| Example 54 | Compound 6 | Au | S-2 | ○ | ○ |
| Example 55 | Compound 6 | Pt | S-1 | ○ | ○ |
| Example 56 | Compound 6 | Pt | S-2 | ○ | ○ |
| Example 57 | Compound 6 | ZrO₂ | S-1 | ○ | ○ |
| Example 58 | Compound 6 | ZrO₂ | S-2 | ○ | ○ |
| Example 59 | Compound 6 | TiO₂ | S-1 | ○ | ○ |
| Example 60 | Compound 6 | TiO₂ | S-2 | ○ | ○ |

TABLE 3

| | Photo- | Material of | | Dispersion | |
|---|---|---|---|---|---|
| | polymerizable | inorganic | | stability | |
| | compound | micro- | Solvent | 1 | 3 |
| | (A) | particles (B) | (S) | month | months |
| Comparative | Comparative | Au | S-1 | X | X |
| Example 3 | Compound | | | | |
| Comparative | Comparative | Au | S-2 | Δ | X |
| Example 4 | Compound | | | | |
| Comparative | Comparative | Pt | S-1 | X | X |
| Example 5 | Compound | | | | |
| Comparative | Comparative | Pt | S-2 | Δ | X |
| Example 6 | Compound | | | | |
| Comparative | Comparative | ZrO₂ | S-1 | Δ | X |
| Example 7 | Compound | | | | |
| Comparative | Comparative | ZrO₂ | S-2 | Δ | X |
| Example 8 | Compound | | | | |
| Comparative | Comparative | TiO₂ | S-1 | Δ | X |
| Example 9 | Compound | | | | |
| Comparative | Comparative | TiO₂ | S-2 | Δ | X |
| Example 10 | Compound | | | | |

Comparison of Examples 13 to 60 with Comparative Examples 3 to 10 shows that the inorganic microparticles (B) are stably dispersed at 40° C. for as long as 3 months in the photosensitive composition including the photopolymerizable compound (A) having a structure corresponding to the formula (A1), while the inorganic microparticles (B) separate or precipitate during 3 months storage at 40° C. in the photosensitive composition including the photopolymerizable compound having a structure not corresponding to the formula (A1).

### Examples 61 to 66, Comparative Example 11, and Comparative Example 12

2.3 parts by mass of the photopolymerizable compound (A) of type shown in Tables 4, 7.5 parts by mass of titanium dioxide microparticles, and 0.2 parts by mass of bis(2,4,6-trimethylbenzoyl)-phenylphosphine oxide as the initiator (C) were dissolved and dispersed in the solvent S-1 (propylene glycol monomethyl ether acetate) so that total mass of the photopolymerizable compound (A), the inorganic microparticles (B), and the initiator (C) was 10% by mass to obtain the photosensitive compositions of Examples 61 to 66, Comparative Example 11, and Comparative Example 11. For the photopolymerizable compound (A), 2-(2-acryloyloxyethyl)oxybiphenyl was used ad comparative compound 1. 4,4′-bis(2-methacryloyloxyethyl)oxydiphenyl sulfide was used as comparative compound 2. According to following methods, light transmittance of the cured film, surface appearance of the cured film, and heat resistance of the cured film were evaluated by using the obtained photosensitive compositions. These evaluation results are shown in Table 4.

### <Evaluation of Light Transmittance of the Cured Film>

The photosensitive composition was coated onto a glass substrate using a spin coater. Subsequently, a film consisting of the photosensitive composition was heated at 110° C. for 2 minutes to obtain a coating film having a thickness to form a cured film having a film thickness of 0.3 μm. The coating film obtained was subjected to light exposure using a high-pressure mercury lamp such that the cumulative exposure amount reached 100 mJ/cm². The exposed coating film was heated at 110° C. for 2 minutes to obtain the cured film having a film thickness of 0.3 μm. Light transmittance of the obtained cured film was measured with a multichannel spectrometer manufactured by Otsuka Electronics Ltd. (MCPD-3000) to determine average transmittance of light of wavelengths from 400 to 700 nm.

### <Evaluation of Surface Appearance>

Surface of the cured film formed in the same manner as in measurement of light transmittance was observed with an optical microscope. Based on the observation result with the optical microscope, surface appearance of the cured product was evaluated according to following criteria.
O: Roughness and/or crack are not observed on the surface.
X: Roughness and/or crack are observed on the surface.

### <Evaluation of Heat Resistance>

An average light transmittance at wavelength range from 400 to 700 nm in the light transmittance evaluation of the cured film above was measured as TO. Subsequently, the cured film was heated at 180 C.° for two minutes. After heating, an average light transmittance at wavelength range from 400 to 700 nm was measured as T1 at a position same as the position for measurement of the average light transmittance TO. Based on the measurement values of TO and T1, a transmittance change rate was calculated according to following formula.

Transmittance change rate (%)=|(T1−T0)/T0×100|

Based on the calculated transmittance change rate, heat resistance of the cured product was evaluated according to following criteria.
O: The transmittance change rate was less than 1%.
X: The transmittance change rate was equal to or more than 1%.

TABLE 4

| | Photo- | Light | | |
|---|---|---|---|---|
| | poly- | transmittance | | |
| | merizable | (Average: | | |
| | compound | 400 nm | Surface | Heat |
| | (A) | to 700 nm) | appearance | resistance |
| Example 61 | Compound 1 | 91 | ◯ | ◯ |
| Example 62 | Compound 2 | 92 | ◯ | ◯ |
| Example 63 | Compound 3 | 92 | ◯ | ◯ |
| Example 64 | Compound 4 | 92 | ◯ | ◯ |
| Example 65 | Compound 5 | 93 | ◯ | ◯ |
| Example 66 | Compound 6 | 93 | ◯ | ◯ |
| Comparative | Comparative | 89 | X | X |
| example 11 | compound 1 | | | |
| Comparative | Comparative | 91 | X | X |
| example 12 | compound 2 | | | |

Comparison of Examples 61 to 66 with Comparative Example 11 and Comparative Example 12 shows that the photosensitive composition including the photopolymerizable compound (A) having a structure corresponding to the formula (A1) gives a cured product with excellent light transmittance, surface appearance, and heat resistance, while the photosensitive composition including the photopolymerizable compound having a structure not corresponding to the formula (A1) gives a cured product with inferior surface appearance and heat resistance.

1. A composition comprising a photopolymerizable compound (A) and inorganic microparticles (B),
wherein the photopolymerizable compound (A) comprises a compound represented by following formula (A¹):
R^{a01}—X^{a03}—R^{a02}—X^{a01}-Ph¹-S-Ph²-X^{a02}—R^{a04}—X^{a04}—R^{a03} (A1),
wherein, in the formula (A¹), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A¹) does not simultaneously have the radically polymerizable group-containing group and the cationically polymerizable group-containing group.

2. The composition according to claim 1, wherein the inorganic microparticles (B) are one or more selected from a group consisting of metal oxide microparticles (B1) and metal microparticles (B2).

3. The composition according to claim 1, wherein the radically polymerizable group-containing group is (meth)acryloyl group, and the cationically polymerizable group-containing group is an epoxy group-containing group.

4. The composition according to claim 1,
wherein the R^{a02} and the R^{a04} are respectively a divalent group consisting of ma aliphatic chain saturated hydrocarbon groups selected from an alkylene group having 1 or more and 4 or less carbon atoms, an alkanetriyl group having 1 or more and 4 or less carbon atoms and an alkyl group having 1 or more and 4 or less carbon atoms, and (ma-1) oxygen atoms and/or sulfur atoms bridging the ma aliphatic chain hydrocarbon groups, and
the ma is an integer of 2 or more and 6 or less.

5. The composition according to claim 4,
wherein the R^{a02} and the R^{a04} are respectively a divalent group consisting of the ma alkylene groups having 1 or more 4 or less carbon atoms, and (ma-1) oxygen atoms and/or sulfur atoms bridging the ma alkylene groups.

6. The composition according to claim 5, wherein the alkylene group is at least one selected from the group consisting of ethane-1,2-diyl group, propane-1,2-diyl group, and propane-1,3-diyl group.

7. The composition according to claim 1,
wherein X^{a01} bonds to para-position in Ph¹ relative to the sulfur atom bonding Ph¹ and Ph², and X^{a02} bonds to para-position in Ph² relative to the sulfur atom bonding Ph¹ and Ph².

8. The composition according to claim 1, wherein the solvent comprises a high boiling point solvent (Si) having boiling point of 170° C. or higher under atmospheric pressure.

9. A photosensitive composition comprising a photopolymerizable compound (A), inorganic microparticles (B), and an initiator (C),
wherein the photopolymerizable compound (A) comprises a compound represented by following formula (A1):
R^{a01}X^{a03}—R^{a02}—X^{a01}-Ph¹-S-Ph²-X^{a02}—R^{a04}—X^{a04}—R^{a03} (A1),
wherein, in the formula (A¹), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A1) does not simultaneously have the radically polymerizable group-containing group and the cationically polymerizable group-containing group.

10. A cured product of the photosensitive composition according to claim 9.

11. A compound represented by following formula (A1):
R^{a01}—X^{a03}—R^{a02}—X^{a01}-Ph¹-S-Ph²-X^{a02}—R^{a04}—X^{a04}—R^{a03} (A1)
wherein, in the formula (A1), R^{a01} and R^{a03} are each independently a radically polymerizable group-containing group or a cationically polymerizable group-containing group,
R^{a02} and R^{a04} are each independently an alkylene group which may be interrupted by one or more oxygen atoms and/or one or more sulfur atoms,
X^{a01}, X^{a02}, X^{a03}, and X^{a04} are each independently oxygen atom or sulfur atom,
Ph¹ and Ph² are each independently a phenylene group optionally substituted with an alkyl group having 1 or more and 5 or less carbon atoms,
sum of a number of oxygen atoms and/or sulfur atoms included in the alkylene group as R^{a02} and a number of oxygen atoms and/or sulfur atoms included in the alkylene group as R^{a04} is 2 or more, and
the compound represented by the formula (A1) does not simultaneously have the radically polymerizable group-containing group and the cationically polymerizable group-containing group, and
provided that, when both of X^{a01} and X^{a02} are sulfur atom, X^{a01} bonds to para-position in Ph¹ relative to the sulfur atom bonding Ph¹ and Ph², and X^{a02} bonds to para-position in Ph² relative to the sulfur atom bonding Ph¹ and Ph², R^{a02} and R^{a04} are not a group represented by —CH₂CH₂—O—CH₂CH₂—.

12. The production method of the compound according to claim 11, comprising, obtaining a compound represented by following formula (A1-d) by reacting a compound represented by following formula (A1-a), a compound represented by following formula (A1-b), and a compound represented by following formula (A1-c), and
substituting hydrogen atom in a terminus represented by —X^{a03}—H with a group represented by R^{a01}, and substituting hydrogen atom in a terminus represented by —X^{a04}—H with a group represented by R^{a03} in the compound represented by the formula (A1-d):
H—X^{a01}-Ph¹-S-Ph²-X^{a02}—H (A1-a)
H—X^{a03}—R^{a02}-Hal (A1-b)
H—X^{a04}—R^{a04}-Hal (A1-c)
H—X^{a03}—R^{a02}—X^{a01}-Ph-S-Ph²-X^{a02}—R^{a04}X^{a04}—H (A1-d),
wherein, in the formula (A1-a), the formula (A1-b), the formula (A1-c), and the formula (A1-d), R^{a01}, R^{a02}, R^{a04}, X^{a01} to X^{a04}, Ph¹, and Ph² are the same as these in the formula (A1), and Hal is a halogen atom.

13. The production method of the compound according to claim 11, comprising, obtaining the compound represented by the formula (A1) by reacting a compound represented by following formula (A1-1a), a compound represented by following formula (A1-e), and a compound represented by following formula (A1-f) in presence of a base:
H—X^{a01}-Ph¹-S-Ph²-X^{a02}—H (A1-a)
R^{a01}—X^{a03}—R^{a02}-Hal (A1-e)
R^{a03}—X^{a04}—R^{a04}-Hal (A1-f)
wherein, in the formula (A1-a), the formula (A1-e), and the formula (A1-f), R^{a01} to R^{a04}, X^{a01} to X^{a04}, Ph¹, and Ph² are the same as these in the formula (A1).
